(19) United States
(12) Patent Application Publication
(10) Pub. No.: US 2021/0317112 A1
(43) Pub. Date: Oct. 14, 2021

(54) HETEROCYCLIC COMPOUNDS AS PESTICIDES

(71) Applicant: Bayer Aktiengesellschaft, Leverkusen (DE)

(72) Inventors: Anne DECOR, Langenfeld (DE); Reiner FISCHER, Monheim (DE); Hans-Georg SCHWARZ, Dorsten (DE); Kerstin ILG, Koeln (DE); Daniela PORTZ, Vettweiss (DE); Sascha EILMUS, Leichlingen (DE); Melanie SCHARWEY, Leichlingen (DE); Marc LINKA, Duesseldorf (DE); Peter LOESEL, Leverkusen (DE); Elke HELLWEGE, Langenfeld (DE); Anthony MILLET, Tignieu-Jameyzieu (FR)

(21) Appl. No.: 16/767,048

(22) PCT Filed: Nov. 26, 2018
(86) PCT No.: PCT/EP2018/082499

§ 371 Date: May 26, 2020

(30) Foreign Application Priority Data
Nov. 28, 2017 (EP) 17203973.7

(51) Int. Cl. C07D 413/12; C07D 417/12; A01N 43/82; A01N 43/80

(52) U.S. Cl. CPC C07D 413/12; A01N 43/80; A01N 43/82; C07D 417/12

(57) ABSTRACT

The present application relates to heterocyclic compounds, compositions containing such compounds, their use for controlling animal pests including arthropods, insects and nematodes, and to processes and intermediates for the preparation of the heterocyclic compounds.

The present application relates to heterocycles compounds of formula (I), compositions containing such compounds, their use for controlling animal pests including arthropods, insects and nematodes, and to processes and intermediates for the preparation of the heterocyclic compounds.

Certain symmetrical 1,3,4-oxadiazol-2-amide compounds as herbicides are disclosed in WO 2015/135946 A1. Cyano isothiazoles are known from WO 2001/040223 A2, WO 2007/116106 A1 and WO 2016/183173 A1. Thiadiazoles as pesticides are disclosed in WO 2017/005717 A1 and WO 2018/108791 A1.

Crop protection compositions, which also include pesticides, have to meet many demands, for example in relation to efficacy, persistence, spectrum, resistance breaking properties, pollinator and beneficial safety of their action and possible use. Questions of toxicity, the combinability with other active compounds or formulation auxiliaries play a role, as well as the question of the expense that the synthesis of an active compound requires. Furthermore, resistances may occur. For all these reasons, the search for novel crop protection compositions cannot be considered to be complete, and there is a constant need for novel compounds having properties which, compared to the known compounds, are improved at least in respect of individual aspects.

It was an object of the present invention to provide compounds which widen the spectrum of the pesticides in various aspects.

This object, and further objects which are not stated explicitly but can be discerned or derived from the connections discussed herein, are achieved by compounds of formula (I) and salts of compounds of formula (I)

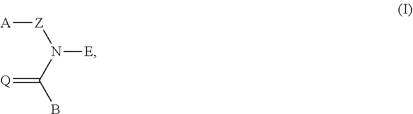

in which
A represents a radical from the group consisting of

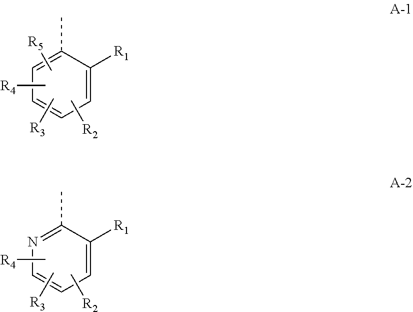

in which the broken line represents the bond to the Z ring,
B represents a radical from the group consisting of

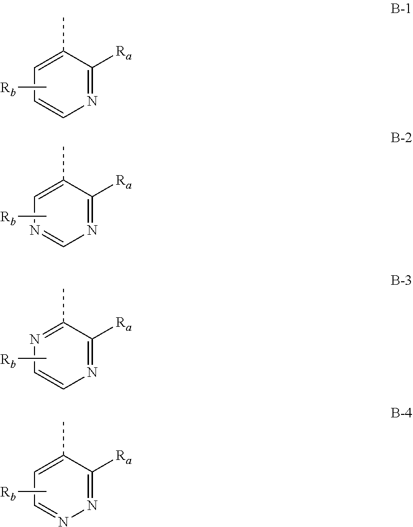

in which the broken line represents the bond to the carbon atom in C=Q,
Z represents a heterocyle from the group consisting of

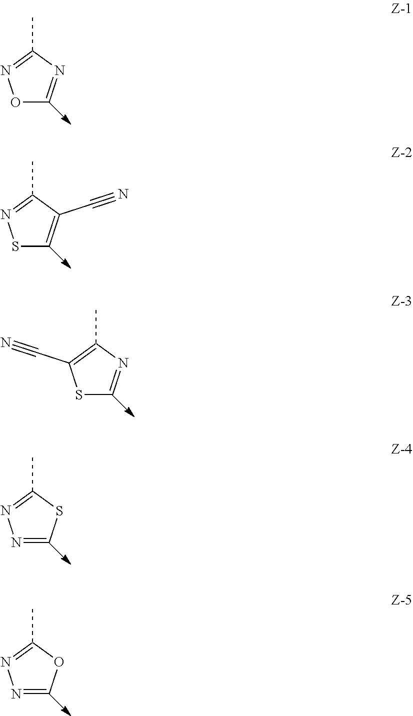

in which the broken line represents the bond to A and the arrow represents the bond to the N(E)C(Q) group,
E is selected from the group consisting of hydrogen, C₁-C₆-alkyl, C₃-C₆-cycloalkyl, C₂-C₆-haloalkyl, C₃-C₆-alkenyl, C₃-C₆-alkynyl, C₃-C₆-cycloalkyl-C₁-C₆-alkyl, cyano-C₁-C₆-alkyl, C₁-C₆-alkylcarbonyl, C₁-C₆-alkoxy-C₁-C₆-alkyl, C₁-C₆-alkoxycarbonyl, a metal ion and an ammonium ion or represents C(═O)—B,
Q represents oxygen or sulfur,
R₁ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-haloalkylthio, C₃-C₆-alkenylthio, C₃-C₆-alkynylthio, C₁-C₆-alkylsulphinyl, C₁-C₆-haloalkylsulphinyl, C₁-C₆-alkylsulphonyl and C₁-C₆-haloalkylsulphonyl,
R₂ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₃ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₄ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₃-C₆-alkenoxy, C₃-C₆-haloalkenoxy, C₃-C₆-alkynoxy, C₃-C₆-cycloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl and
R_{b} is selected from the group consisting of hydrogen, halogen, nitro, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl.

It has been found that compounds of the formula (I) have pronounced biological properties and are suitable especially for controlling animal pests, in particular insects and arachnids, encountered in agriculture, in forests, in the protection of stored products and materials and in the hygiene sector and for controlling arthropodal parasites on animals, in particular warm-blooded animals.

Preferred are compounds of formula (I) in which
A represents a radical from the group consisting of R5

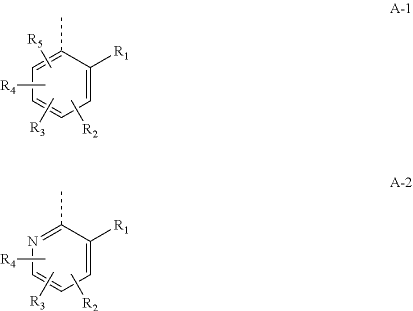

in which the broken line represents the bond to the Z ring,
B represents a radical from the group consisting of

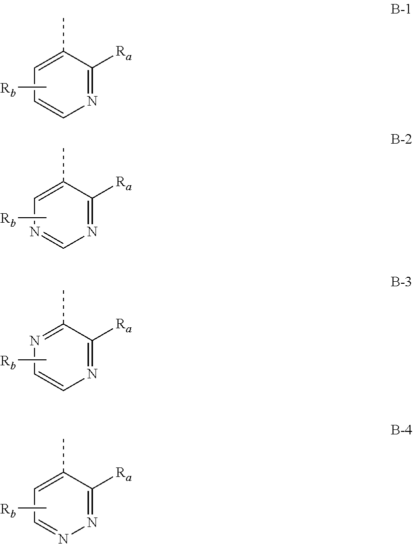

in which the broken line represents the bond to the carbon atom in C=Q,
Z represents a radical from the group consisting of

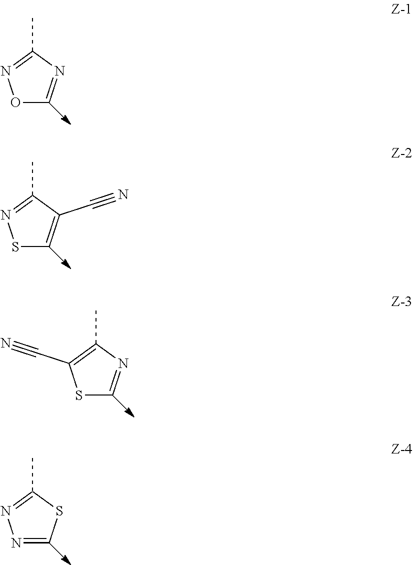

in which the broken line represents the bond to A and the arrow represents the bond to the N(E)C(Q) group,
E is selected from the group consisting of hydrogen, C₁-C₄-alkyl, C₂-C₄-haloalkyl, cyano-C₁-C₄-alkyl, C₁-C₄-alkylcarbonyl, C₁-C₄-alkoxy-C₁-C₄-alkyl, C₁-C₄-alkoxycarbonyl, an alkali metal ion, an earth alkali metal ion and an ammonium ion or represents C(═O)—B,
Q represents oxygen or sulfur,
R₁ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-haloalkylthio, C₃-C₆-alkenylthio, C₃-C₆-alkynylthio, C₁-C₆-alkylsulphinyl, C₁-C₆-haloalkylsulphinyl, C₁-C₆-alkylsulphonyl and C₁-C₆-haloalkylsulphonyl,
R₂ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₃ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₄ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₃-C₆-alkenoxy, C₃-C₆-haloalkenoxy, C₃-C₆-alkynoxy, C₃-C₆-cycloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl and
R_{b} is selected from the group consisting of hydrogen, halogen, nitro, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl.

Particularly preferred are compounds of the formula (I) in which
A represents a radical from the group consisting of

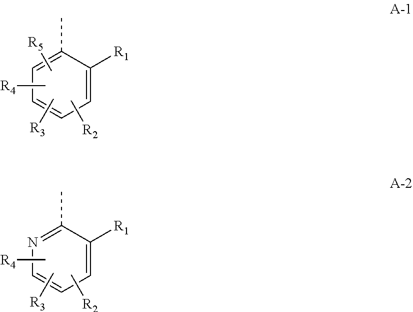

in which the broken line represents the bond to the Z ring,
B represents a radical from the group consisting of

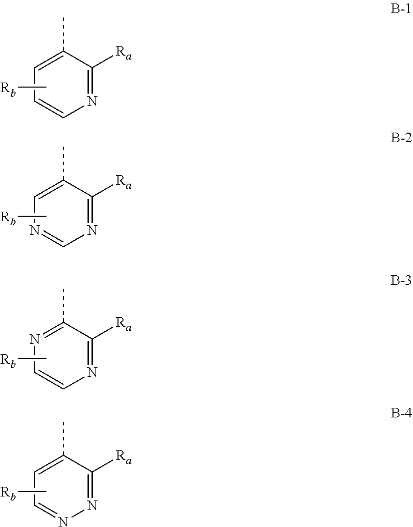

in which the broken line represents the bond to the carbon atom in C=Q,
Z represents a heterocycle from the group consisting of

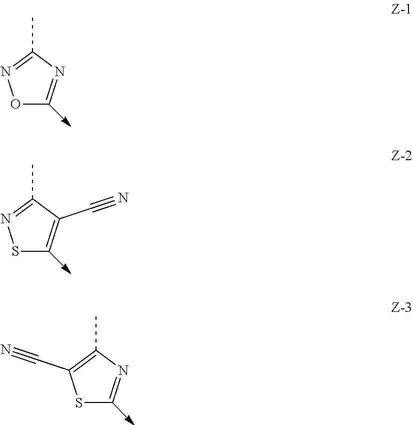

in which the broken line represents the bond to A and the arrow represents the bond to the N(E)C(Q) group,
E is selected from the group consisting of hydrogen, C₁-C₄-alkyl, C₂-C₄-haloalkyl, cyano-C₁-C₄-alkyl, C₁-C₄-alkylcarbonyl, C₁-C₄-alkoxy-C₁-C₄-alkyl, C₁-C₄-alkoxycarbonyl, an alkali metal ion, an earth alkali metal ion and an ammonium ion or represents C(═O)—B,
Q represents oxygen or sulfur,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, -difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Very particularly preferred are also compounds of the formula (I) in which
A represents a radical from the group consisting of

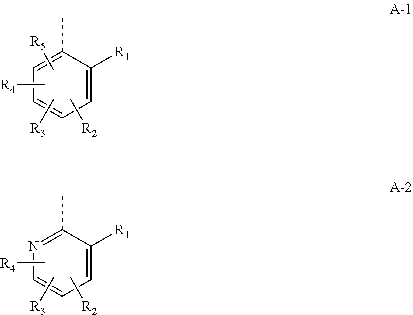

in which the broken line represents the bond to the Z ring,
B represents a radical from the group consisting of

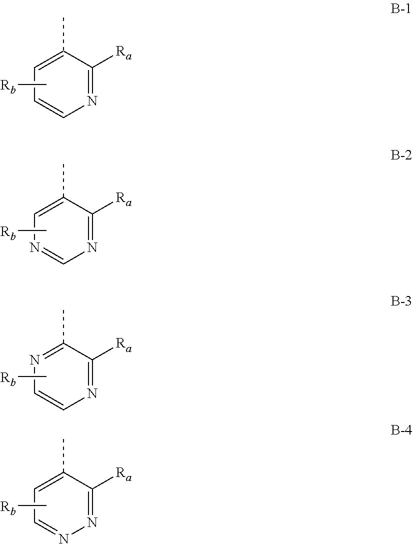

in which the broken line represents the bond to the carbon atom in C=Q,
Z represents a heterocycle from the group consisting of

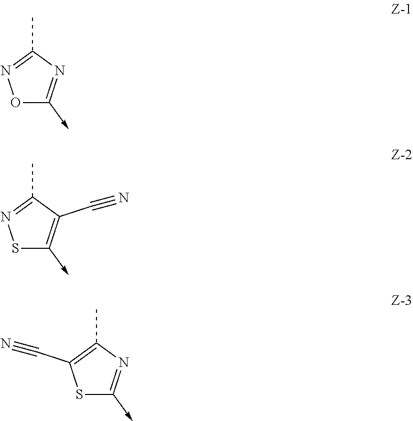

in which the broken line represents the bond to A and the arrow represents the bond to the N(E)C(Q) group,
E is selected from the group consisting of hydrogen, methyl, ethyl, a Li-, Na-, K-, Mg- and Ca-ion, or
E represents C(═O)—B,
Q represents oxygen,
R₁ is selected from the group consisting of fluorine, chlorine, bromine, iodine, cyano, methyl, ethyl, n-propyl, i-propyl, difluoromethyl, trifluoromethyl, chloro-difluoromethyl and pentafluoroethyl,
R₂ is selected from the group consisting of hydrogen, fluorine, chlorine, bromine, iodine, cyano, difluoromethyl, trifluoromethyl, chloro-difluoromethyl and pentafluoroethyl,
R₃ is selected from the group consisting of hydrogen, fluorine, chlorine, bromine, iodine, cyano, difluoromethyl, trifluoromethyl, chloro-difluoromethyl and pentafluoroethyl,
R₄ is selected from the group consisting of hydrogen, fluorine, chlorine, bromine, iodine, cyano, difluoromethyl, trifluoromethyl, chloro-difluoromethyl and pentafluoroethyl,
R₅ is selected from the group consisting of hydrogen, fluorine, chlorine, bromine, iodine, cyano, difluoromethyl, trifluoromethyl, chloro-difluoromethyl and pentafluoroethyl,
Rₐ is selected from the group consisting of fluorine, chlorine, bromine, iodine, nitro, methyl, ethyl, difluoromethyl, trifluoromethyl, chloro-difluoromethyl, pentafluoroethyl, methoxy and ethoxy and
R_{b} is selected from the group consisting of hydrogen, fluorine, chlorine, bromine and iodine.

A particularly preferred group of compounds are compounds of formula (I-1)

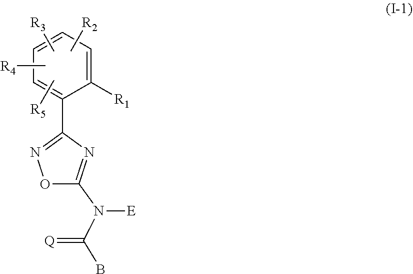

in which
B represents B-1

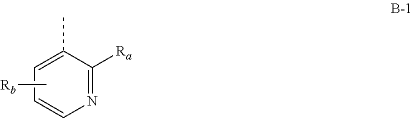

in which the broken line represents the bond to the carbon atom in C=Q,
E is selected from the group consisting of hydrogen a Li-, Na-, K-, Mg-, Ca-ion and an ammonium ion of formula

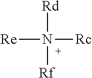

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-1)

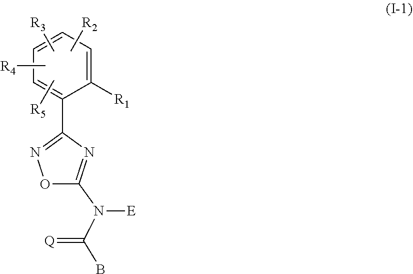

in which
B represents a radical from the group consisting of

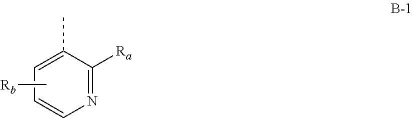

in which the broken line represents the bond to the carbon atom in C=Q,
E represents hydrogen,
Q represents oxygen,
R₁ is selected from the group consisting of fluorine, chlorine, bromine, iodine, cyano, methyl, ethyl, n-propyl, i-propyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy and trifluoroethoxy,
R₂ is selected from the group consisting of hydrogen, fluorine, chlorine, bromine, iodine, cyano, difluoromethyl, trifluoromethyl, chloro-difluoromethyl and pentafluoroethyl,
R₃ is selected from the group consisting of fluorine, chlorine, bromine, iodine, cyano, difluoromethyl, trifluoromethyl, chloro-difluoromethyl and pentafluoroethyl,
R₄ is selected from the group consisting of hydrogen, fluorine, chlorine, bromine, iodine, cyano, difluoromethyl, trifluoromethyl, chloro-difluoromethyl and pentafluoroethyl,
R₅ is selected from the group consisting of hydrogen, fluorine, chlorine, bromine, iodine, cyano, difluoromethyl, trifluoromethyl, chloro-difluoromethyl and pentafluoroethyl,
Rₐ is selected from the group consisting of fluorine, chlorine, bromine, iodine, nitro, methyl, ethyl, difluoromethyl, trifluoromethyl, chloro-difluoromethyl, pentafluoroethyl, methoxy and ethoxy, and
R_{b} is selected from the group consisting of hydrogen, fluorine, chlorine, bromine and iodine.

Another particularly preferred group of compounds are compounds of formula (I-1)

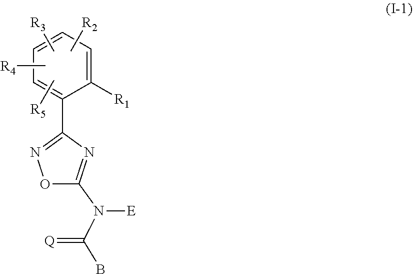

in which
B represents a radical from the group consisting of

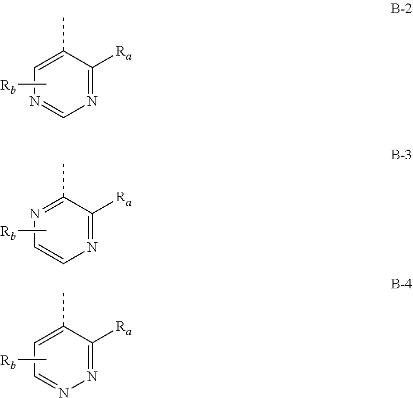

in which the broken line represents the bond to the carbon atom in C=Q,
E is selected from the group consisting of hydrogen, a Li-, Na-, K-, Mg-, Ca-ion and an ammonium ion of formula

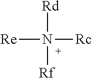

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-1)

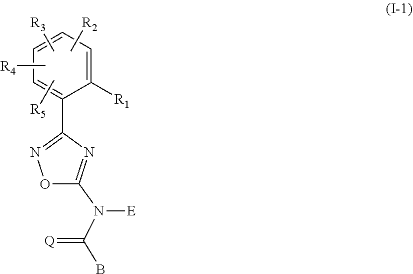

in which
B represents a radical from the group consisting of

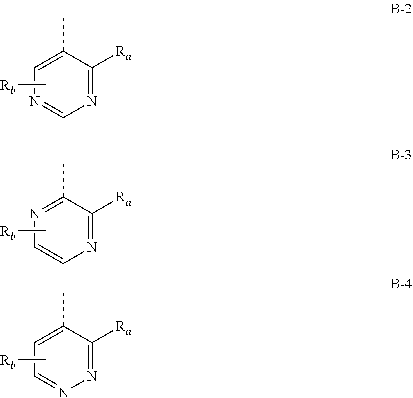

in which the broken line represents the bond to the carbon atom in C=Q,
E represents hydrogen,
Q represents oxygen,
R₁ is selected from the group consisting of fluorine, chlorine, bromine, iodine, cyano, methyl, ethyl, n-propyl, i-propyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy and trifluoroethoxy,
R₂ is selected from the group consisting of hydrogen, fluorine, chlorine, bromine, iodine, cyano, difluoromethyl, trifluoromethyl, chloro-difluoromethyl and pentafluoroethyl,
R₃ is selected from the group consisting of hydrogen, fluorine, chlorine, bromine, iodine, cyano, difluoromethyl, trifluoromethyl, chloro-difluoromethyl and pentafluoroethyl,
R₄ is selected from the group consisting of hydrogen, fluorine, chlorine, bromine, iodine, cyano, difluoromethyl, trifluoromethyl, chloro-difluoromethyl and pentafluoroethyl,
R₅ is selected from the group consisting of hydrogen, fluorine, chlorine, bromine, iodine, cyano, difluoromethyl, trifluoromethyl, chloro-difluoromethyl and pentafluoroethyl,
Rₐ is selected from the group consisting of fluorine, chlorine, bromine, iodine, nitro, methyl, ethyl, difluoromethyl, trifluoromethyl, chloro-difluoromethyl, pentafluoroethyl, methoxy and ethoxy, and
R_{b} is selected from the group consisting of hydrogen, fluorine, chlorine, bromine and iodine.

Another particularly preferred group of compounds are compounds of formula (I-1)

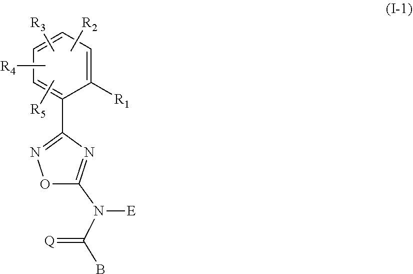

in which
B represents a radical from the group consisting of

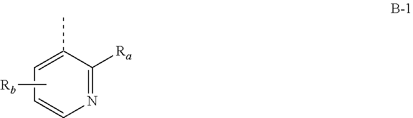

in which the broken line represents the bond to the carbon atom in C=Q,
E represents hydrogen,
Q represents oxygen,
R₁ is selected from the group consisting of fluorine, chlorine, bromine, and iodine,
R₂ is selected from the group consisting of hydrogen, fluorine, chlorine, bromine, iodine, methyl, ethyl, difluoromethyl, trifluoromethyl, chloro-difluoromethyl and pentafluoroethyl,
R₃ is selected from the group consisting of hydrogen, fluorine, chlorine, bromine, and iodine,
R₄ is selected from the group consisting of hydrogen, fluorine, chlorine, bromine and iodine,
R₅ represents hydrogen,
Rₐ is selected from the group consisting of methyl, ethyl, difluoromethyl, trifluoromethyl, chloro-difluoromethyl, pentafluoroethyl, and
R_{b} represents hydrogen.

Another particularly preferred group of compounds are compounds of formula (I-1)

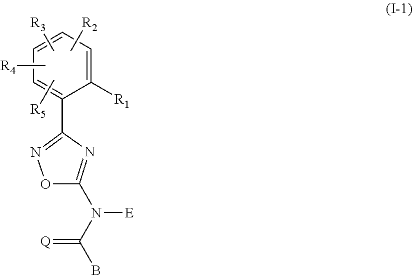

in which
B represents a radical from the group consisting of

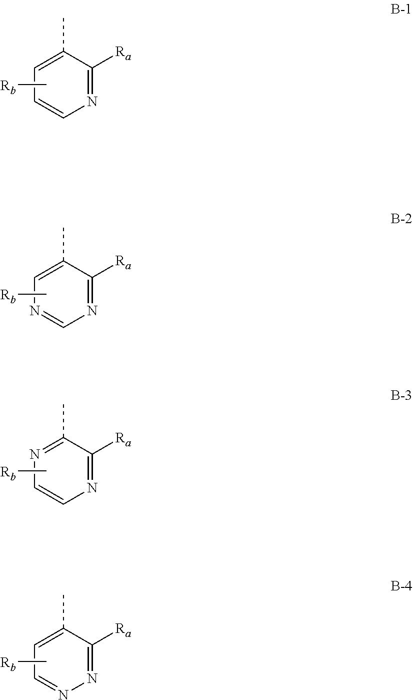

in which the broken line represents the bond to the carbon atom in C=Q,
E represents hydrogen,
Q represents oxygen,
R₁ is selected from the group consisting of fluorine, chlorine, bromine, and iodine,
R₂ is selected from the group consisting of hydrogen, fluorine, chlorine, bromine, iodine, methyl, ethyl, difluoromethyl, trifluoromethyl, chloro-difluoromethyl and pentafluoroethyl,
R₃ is selected from the group consisting of hydrogen, fluorine, chlorine, bromine, and iodine,
R₄ is selected from the group consisting of hydrogen, fluorine, chlorine, bromine and iodine,
R₅ represents hydrogen,
Rₐ is selected from the group consisting of methyl, ethyl, difluoromethyl, trifluoromethyl, chloro-difluoromethyl, pentafluoroethyl, and
R_{b} represents hydrogen.

Another particularly preferred group of compounds are compounds of formula (I-2)

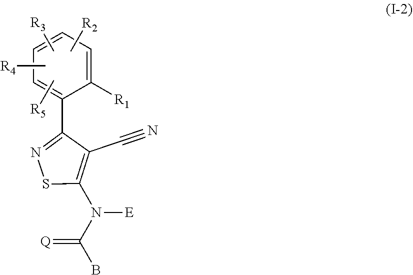

in which
B represents a radical from the group consisting of

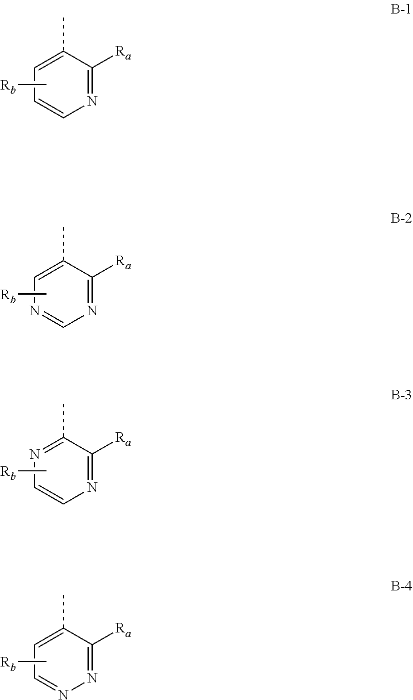

in which the broken line represents the bond to the carbon atom in C=Q,
E is selected from the group consisting of hydrogen, a Li-, Na-, K-, Mg-, Ca-ion and an ammonium ion of formula

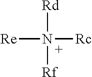

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-2)

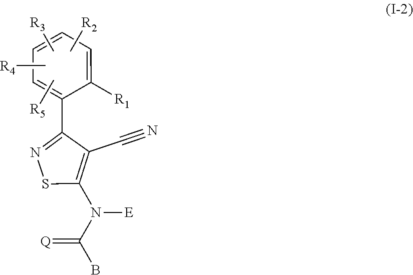

in which
B represents B-1

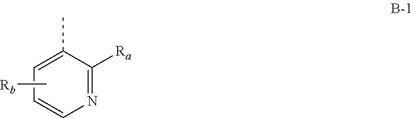

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

Another particularly preferred group of compounds are compounds of formula (I-2)

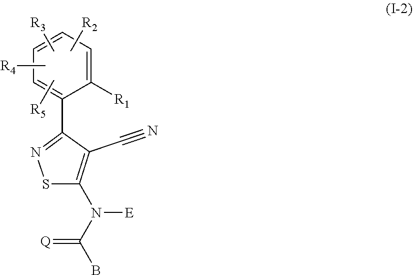

in which
B represents B-3

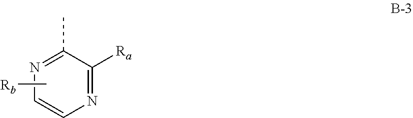

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

Another particularly preferred group of compounds are compounds of formula (I-3)

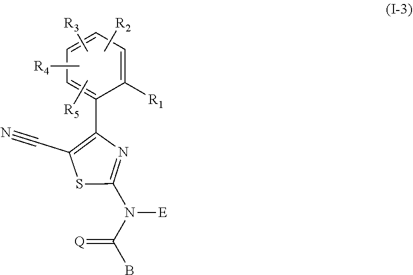

in which
B represents a radical from the group consisting of

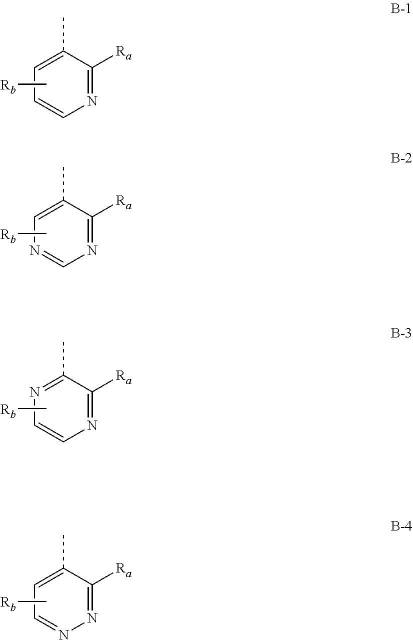

in which the broken line represents the bond to the carbon atom in C=Q,
E is selected from the group consisting of hydrogen, a Li-, Na-, K-, Mg-, Ca-ion and an ammonium ion of formula

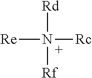

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-3)

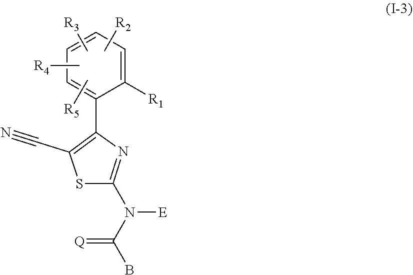

in which
B represents B-1

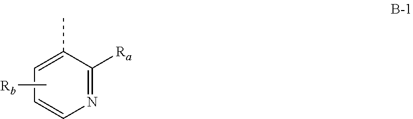

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

Another particularly preferred group of compounds are compounds of formula (I-3)

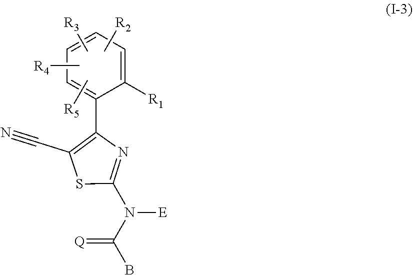

in which
B represents B-3

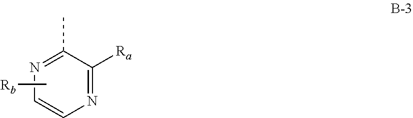

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

Another particularly preferred group of compounds are compounds of formula (I-4)

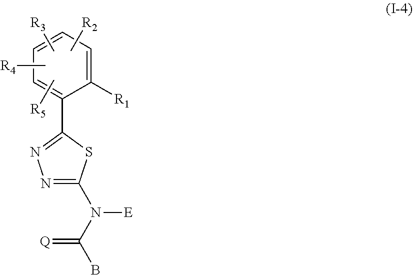

in which
B represents a radical from the group consisting of

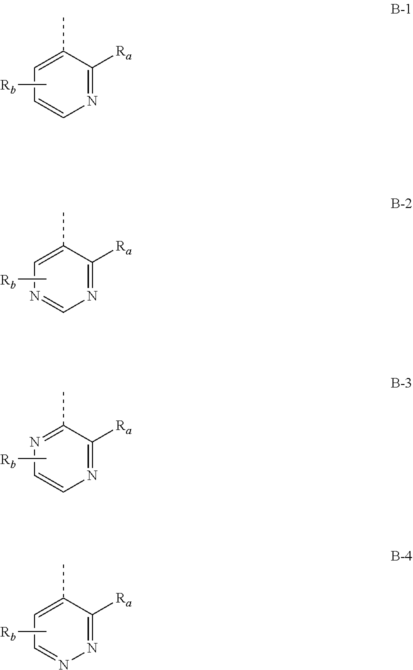

in which the broken line represents the bond to the carbon atom in C=Q,
E is selected from the group consisting of hydrogen, a Li-, Na-, K-, Mg-, Ca-ion and an ammonium ion of formula

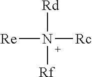

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-4)

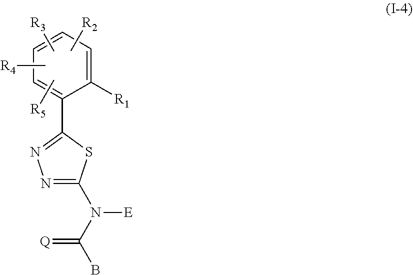

in which
B represents B-1

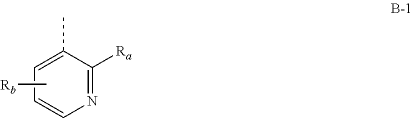

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

Another particularly preferred group of compounds are compounds of formula (I-5)

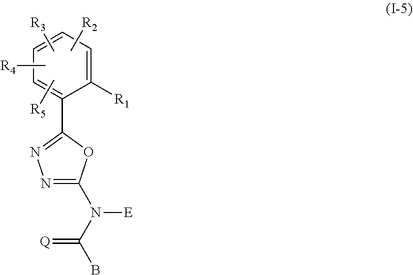

in which
B represents a radical from the group consisting of

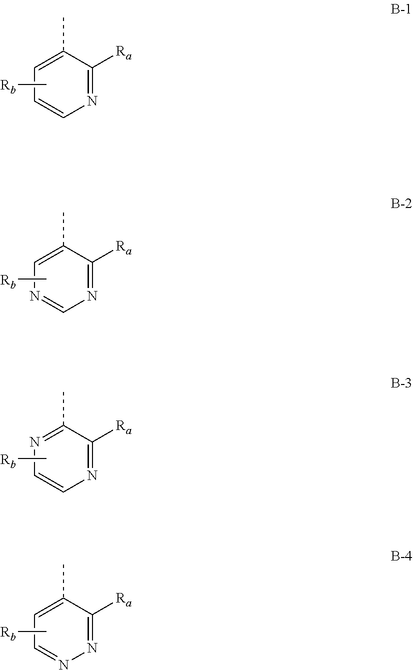

in which the broken line represents the bond to the carbon atom in C=Q,
E is selected from the group consisting of hydrogen, a Li-, Na-, K-, Mg-, Ca-ion and an ammonium ion of formula

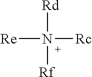

wherein Rc, Rd, Re and Rf independently represent hydrogen, methyl, ethyl or benzyl,
Q represents oxygen,
R₁ is selected from the group consisting of fluorine, chlorine, bromine, iodine, cyano, nitro, methyl, ethyl, n-propyl, i-propyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₂ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₃ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₄ is selected from the group consisting of hydrogen, fluorine, chlorine, bromine, iodine, cyano, nitro, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of fluorine, chlorine, bromine, iodine, cyano, nitro, methyl, ethyl, difluoromethyl, trifluoromethyl, chloro-difluoromethyl, pentafluoroethyl, cyclopropyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, methylsulphinyl, ethylsulphinyl, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl, and
R_{b} is selected from the group consisting of hydrogen, fluorine, chlorine, bromine, iodine, nitro, cyano, methyl, ethyl, difluoromethyl, trifluoromethyl, chloro-difluoromethyl, pentafluoroethyl, methoxy, ethoxy, difluoromethoxy, trifluoromethoxy, chloro-difluoro-methoxy, difluoroethoxy, trifluoroethoxy, methylthio, ethylthio, trifluoromethylthio, difluoromethylthio, difluoroethylthio, trifluoroethylthio, trifluoromethylsulphinyl, difluoromethylsulphinyl, difluoroethylsulphinyl, trifluoroethylsulphinyl, methylsulphonyl, ethylsulphonyl, trifluoromethylsulphonyl, difluoromethylsulphonyl, difluoroethylsulphonyl and trifluoroethylsulphonyl.

Another particularly preferred group of compounds are compounds of formula (I-5)

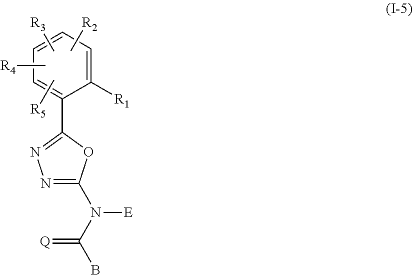

in which
B represents B-1

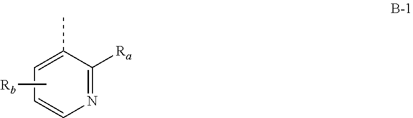

in which the broken line represents the bond to the carbon atom in C=Q,
E represents hydrogen,
Q represents oxygen,
R₁ is selected from the group consisting of fluorine, chlorine, bromine and iodine,
R₂ is selected from the group consisting of hydrogen, fluorine, chlorine, bromine, iodine, difluoromethyl, trifluoromethyl, chloro-difluoromethyl and pentafluoroethyl,
R₃ is selected from the group consisting of hydrogen, fluorine, chlorine, bromine, iodine, difluoromethyl, trifluoromethyl, chloro-difluoromethyl and pentafluoroethyl,
R₄ is selected from the group consisting of hydrogen, fluorine, chlorine, bromine, iodine, difluoromethyl, trifluoromethyl, chloro-difluoromethyl and pentafluoroethyl,
R₅ is selected from the group consisting of hydrogen, fluorine, chlorine, bromine, iodine, difluoromethyl, trifluoromethyl, chloro-difluoromethyl and pentafluoroethyl,
Rₐ is selected from the group consisting of fluorine, chlorine, bromine, iodine, difluoromethyl, trifluoromethyl, chloro-difluoromethyl and pentafluoroethyl, and
R_{b} represents hydrogen.

In the above definitions, unless stated otherwise, halogen is selected from the group of fluorine, chlorine, bromine and iodine, preferably in turn from the group of fluorine, chlorine and bromine.

Halogen-substituted radicals, for example haloalkyl, are mono- or polyhalogenated, up to the maximum number of possible substituents. In the case of polyhalogenation, the halogen atoms may be identical or different. Halogen denotes fluorine, chlorine, bromine and iodine, in particular fluorine, chlorine and bromine.

Saturated or unsaturated hydrocarbyl radicals, such as alkyl or alkenyl, may in each case be straight-chain or branched as far as possible, including in combination with heteroatoms, as, for example, in alkoxy.

Optionally substituted radicals may be mono- or polysubstituted, where the substituents in the case of polysubstitution may be the same or different.

The radical definitions or elucidations given above in general terms or within areas of preference apply to the end products and correspondingly to the starting materials and intermediates. These radical definitions can be combined with one another as desired, i.e. including combinations between the respective preferred ranges.

In a preferred group of compounds of formula (I) A represents A-1.

In another preferred group of compounds of formula (I) A represents A-2.

In another preferred group of compounds of formula (I) B represents B-1.

In another preferred group of compounds of formula (I) B represents B-2.

In another preferred group of compounds of formula (I) B represents B-3.

In another preferred group of compounds of formula (I) B represents B-4.

In another preferred group of compounds of formula (I) Z represents Z-1.

In another preferred group of compounds of formula (I) Z represents Z-2.

In another preferred group of compounds of formula (I) Z represents Z-3.

In another preferred group of compounds of formula (I) A represents A-1 and B represents B-1.

In another preferred group of compounds of formula (I) A represents A-1, B represents B-1 and R₄, R₅, and R_{b} represent hydrogen.

In another preferred group of compounds of formula (I) A represents A-1 and B represents B-2.

In another preferred group of compounds of formula (I) A represents A-1 and B represents B-3.

In another preferred group of compounds of formula (I) A represents A-1, B represents B-3 and R₄, R₅, and R_{b} represent hydrogen.

In another preferred group of compounds of formula (I) A represents A-1, Z represents Z-2, B represents B-1, Q represents oxygen, and E, R₃, R₄, R₅, and R_{b} represent hydrogen.

In another preferred group of compounds of formula (I) A represents A-1, Z represents Z-2, B represents B-3, Q represents oxygen, and E, R₃, R₄, R₅, and R_{b} represent hydrogen.

In another preferred group of compounds of formula (I) A represents A-1, Z represents Z-3, B represents B-1, Q represents oxygen, and E, R₃, R₄, R₅, and R_{b} represent hydrogen.

In another preferred group of compounds of formula (I) A represents A-1, Z represents Z-3, B represents B-3, Q represents oxygen, and E, R₃, R₄, R₅, and R_{b} represent hydrogen.

In another preferred group of compounds of formula (I) A represents A-1, Z represents Z-2, B represents B-1, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

In another preferred group of compounds of formula (I) A represents A-1, Z represents Z-2, B represents B-3, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

In another preferred group of compounds of formula (I) A represents A-1, Z represents Z-3, B represents B-1, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

In another preferred group of compounds of formula (I) A represents A-1, Z represents Z-3, B represents B-3, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

In another preferred group of compounds of formula (I) A represents A-1, Z represents Z-5, B represents B-1, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

In another preferred group of compounds of formula (I) A represents A-2 and B represents B-1.

In another preferred group of compounds of formula (I) A represents A-2 and B represents B-3.

It has additionally been found that compounds of the formula (I) can be obtained by the processes described below.

Accordingly, the invention also relates to processes for preparing compounds of the formula (I) in which Q represents oxygen and E represents hydrogen. In the groups of compounds below, the individual radicals A and B have the meanings given above.

Synthesis Scheme 1 depicts the general route of synthesizing the final carboxamides (I).

Compounds of formula (I) wherein E represents hydrogen and Q represents oxygen can be obtained from the amine of formula (II) either by amide coupling with a carboxylic acid of formula (III) or by reaction with an acyl chloride of formula (IV).

In case of the amide reaction (Route A), in addition to the acid (III) and the amine (II) a base such as ethyl-N,N-diisopropylamine and a coupling agent such as 1-[bis(dimethylamino)methylene]-1H-1,2,3-triazolo[4,5-b]pyridinium 3-oxid hexafluoro phosphate) (HATU) can be used in a solvent such as dimethylformamide with or without a catalytic amount of 4-(N,N-dimethylamino)pyridine, at a temperature of e.g. 80° C.

In case of the reaction with an acyl chloride (Route B) a base is used in systems such as triethylamine in dichloromethane at room temperature,

Acyl chlorides of formula (IV) are commercially available or can be prepared from the corresponding carboxylic acids of formula (III) using a chlorinating agent such as thionyl chloride in a solvent such as toluene.

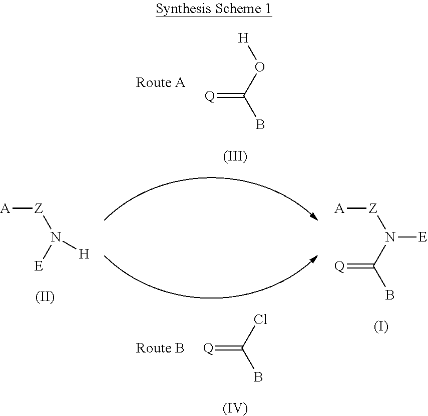

Synthesis Scheme 2 depicts the way of synthesizing the precursor of amines (II-1) and synthesizing other amines (II) wherein A represents A-1 or A-2.

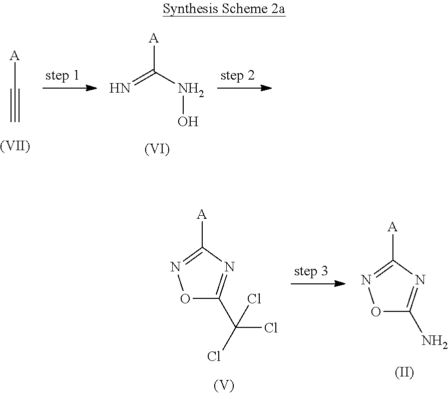

Step 1: Synthesis Scheme 2a shows that nitriles of formula (VII) can react with hydroxylamine hydrochloride in presence of a base such as triethylamine or potassium carbonate or sodium hydrogenocarbonate leading to hydroxyamidines (VI) with heating (e.g. at 70° C. or 65° C.), in a solvent such as ethanol, or methanol.

Step 2: At a temperature such as 120° C. hydroxyamidines of formula (VI) can be cyclized in the presence of trichloroacetic anhydride to give intermediates of formula (V).

Step 3: Intermediates of formula (V) can be converted to amines of formula (II) using ammonia in a solvent such as methanol, at elevated temperatures (e.g. 50° C.). Analogous conversions are known from WO 2007/099326.

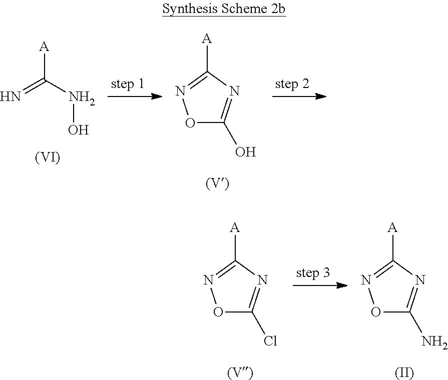

Step 1: Hydroxylamine of formula (VI) can react with carbonyldiimidazole in a solvent such as acetonitrile by heating e.g. at reflux to give cyclic intermediate (V′)

Step 2: Intermediate of formula (V′) can be converted to chloro-electrophile of formula (V″) using phosphorus oxychloride under heating e.g. at 100° C.

Step 3: Intermediates of formula (V″) can be converted to amines of formula (II) using ammonium hydroxide in a solvent such as dioxane. Analogous conversions are known from U.S. Pat. No. 4,642,312,

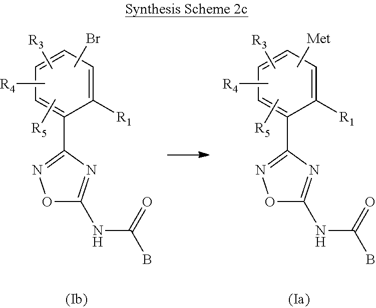

Methylated compound of formula (Ia) can be obtained from brominated precursor of formula (Ib) by Suzuki coupling using methyl boronic acid, a base such as potassium carbonate and a palladium source such as Pd.dppf.Cl₂.CH₂Cl₂, heating at e.g. 130° C. in a solvent such as dioxane/water.

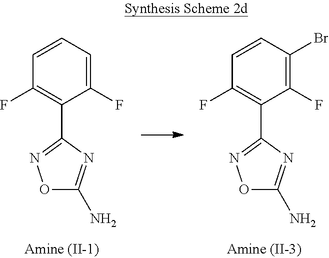 [SMILES: Nc1nc(-c2c(F)cccc2F)no1 ; Nc1nc(-c2c(F)ccc(Br)c2F)no1]

Novel amine of formula (II-3) [(3-bromo-2,6-difluorophenyl)-1,2,4-oxadiazol-5-amine] can be obtained from amine of formula (II-1) [3-(2,6-difluorophenyl)-1,2,4-oxadiazol-5-amine] using a brominating agent such as N-bromosuccinimide in an acidic solvent such as trifluoro acetic acid, heating for example at 60° C.

Synthesis Scheme 3 depicts the way of synthesizing the amines (VII) and their precursors.

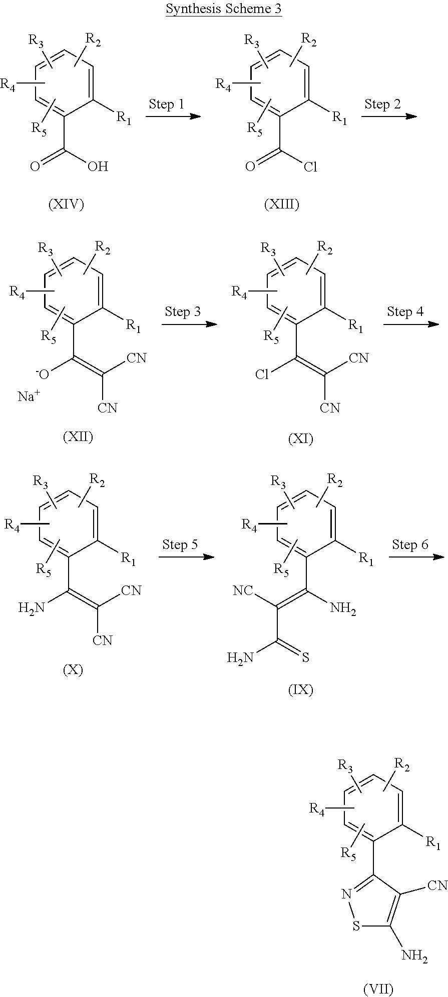

An analogous synthetic sequence from step 2 to step 6 is known from Bioorganic and Medicinal Chemistry Letters, 2012 vol. 22, (9), 3223-3228.

Step 1: Synthesis Scheme 3 shows that carboxylic acids of formula (XIV) can be transformed into their corresponding acyl chloride of formula (XIII) using for example thionly chloride.

Step 2: Acyl chlorides of formula (XIII) can be functionalized to give intermediates of formula (XII) using malonitrile and a base such as sodium hydride in a solvent such as tetrahydrofurane for example at room temperature.

Step 3: Intermediates of formula (XII) can be converted to intermediates of formula (XI) using an agent such as POCl₃ in a solvent such dichloromethane in presence of a base, e.g. triethylamine Step 4: Chlorides of formula (XI) can be converted to amino-intermediates of formula (X), using ammonia, for example in methanol, e.g. at room temperature.

Step 5: Amino-intermediates of formula (X) can be converted to intermediates of formula (IX) using H₂S in presence of a base such as trimethylamine in a solvent such as pyridine, for example at room temperature.

Step 6: Intermediates of formula (IX) can be cyclized in the presence of aqueous hydrogen peroxide in a solvent such as methanol, heating e.g. at 50° C., to give amines of formula (VII).

Synthesis Scheme 4 depicts the way of synthesizing the amines (XV) and their precursors.

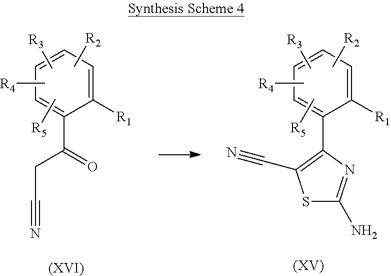

Amines of formula (XV) can be prepared starting from oxo-nitrile precursors of formula (XVI), using thiourea, pyridine and iodine, at elevated temperature (e.g. 100° C.). Analogous cyclizations are known from Bioorganic & Medicinal Chemistry Letters, 20 (17), 5241-5244; 2010. Some oxo-nitrile precursors of formula (XVI) are commercially available.

The following novel amines have been synthesized according to Synthesis Scheme 3 and 4 respectively.

Amine (VII-2): 5-amino-3-[2-(trifluoromethyl)phenyl]-1,2-thiazole-4-carbonitrile

Amine (XV-2): 2-amino-4-[2-(trifluoromethyl)phenyl]-1,3-thiazole-5-carbonitrile Novel Amine II-3 (3-bromo-2,6-difluorophenyl)-1,2,4-oxadiazol-5-amine was synthesized according to Synthesis 2d.

The processes according to the invention for the preparation of compounds of the formula (I) are preferably performed using a diluent. Useful diluents for performance of the processes according to the invention are, as well as water, all inert solvents. Examples include: halohydrocarbons (for example chlorohydrocarbons such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene), alcohols (for example methanol, ethanol, isopropanol, butanol), ethers (for example ethyl propyl ether, methyl tert-butyl ether, anisole, phenetole, cyclohexyl methyl ether, dimethyl ether, diethyl ether, dipropyl ether, diisopropyl ether, di-n-butyl ether, diisobutyl ether, diisoamyl ether, ethylene glycol dimethyl ether, tetrahydrofuran, 1,4-dioxane, dichlorodiethyl ether and polyethers of ethylene oxide and/or propylene oxide), amines (for example trimethyl-, triethyl-, tripropyl-, tributylamine, N-methylmorpholine, pyridine and tetramethylenediamine), nitrohydrocarbons (for example nitromethane, nitroethane, nitropropane, nitrobenzene, chloronitrobenzene, o-nitrotoluene); nitriles (for example acetonitrile, propionitrile, butyronitrile, isobutyronitrile, benzonitrile, m-chlorobenzonitrile), tetrahydrothiophene dioxide, dimethyl sulphoxide, tetramethylene sulphoxide, dipropyl sulphoxide, benzyl methyl sulphoxide, diisobutyl sulphoxide, dibutyl sulphoxide, diisoamyl sulphoxide, sulphones (for example dimethyl, diethyl, dipropyl, dibutyl, diphenyl, dihexyl, methyl ethyl, ethyl propyl, ethyl isobutyl and pentamethylene sulphone), aliphatic, cycloaliphatic or aromatic hydrocarbons (for example pentane, hexane, heptane, octane, nonane and technical hydrocarbons), and also what are called “white spirits” with components having boiling points in the range from, for example, 40° C. to 250° C., cymene, petroleum fractions within a boiling range from 70° C. to 190° C., cyclohexane, methylcyclohexane, petroleum ether, ligroin, benzene, toluene, xylene, esters (for example methyl, ethyl, butyl and isobutyl acetate, dimethyl, dibutyl and ethylene carbonate); amides (for example hexamethylphosphoric triamide, formamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine, N,N′-diformylpiperazine) and ketones (for example acetone, acetophenone, methyl ethyl ketone, methyl butyl ketone).

It is also possible to perform the process according to the invention in mixtures of the solvents and diluents mentioned.

When performing the process according to the invention, the reaction temperatures can be varied within a relatively wide range. In general, the temperatures employed are between −30° C. and +150° C., preferably between −10° C. and +100° C.

The process according to the invention is generally performed under atmospheric pressure. However, it is also possible to perform the process according to the invention under elevated or reduced pressure—generally at absolute pressures between 0.1 bar and 15 bar.

To perform the process according to the invention, the starting materials are generally used in approximately equimolar amounts. However, it is also possible to use one of the components in a relatively large excess. The reaction is generally carried out in a suitable diluent in the presence of a reaction auxiliary, optionally also under a protective gas atmosphere (for example under nitrogen, argon or helium) and the reaction mixture is generally stirred at the temperature required for several hours. The workup is performed by customary methods (cf. the preparation examples).

The basic reaction auxiliaries used to perform the process according to the invention may be all suitable acid binders. Examples include: alkaline earth metal or alkali metal compounds (e.g. hydroxides, hydrides, oxides and carbonates of lithium, sodium, potassium, magnesium, calcium and barium), amidine bases or guanidine bases (e.g. 7-methyl-1,5,7-triazabicyclo[4.4.0]dec-5-ene (MTBD); diazabicyclo[4.3.0]nonene (DBN), diazabicyclo[2.2.2]octane (DABCO), 1,8-diazabicyclo[5.4.0]undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine) and amines, especially tertiary amines (e.g. triethylamine, trimethylamine, tribenzylamine, triisopropylamine, tributylamine, tricyclohexylamine, triamylamine, trihexylamine, N,N-dimethylaniline, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine, pyridine, 4-pyrrolidinopyridine, 4-dimethylaminopyridine, quinoline, 2-picoline, 3-picoline, pyrimidine, acridine, N,N,N′,N′-tetramethylenediamine, N,N,N′,N′-tetraethylenediamine, quinoxaline, N-propyldiisopropylamine, N-ethyldiisopropylamine, N,N′-dimethylcyclohexylamine, 2,6-lutidine, 2,4-lutidine or triethylenediamine).

The acidic reaction auxiliaries used to perform the process according to the invention include all mineral acids (e.g. hydrohalic acids such as hydrofluoric acid, hydrochloric acid, hydrobromic acid or hydriodic acid, and also sulphuric acid, phosphoric acid, phosphorous acid, nitric acid), Lewis acids (e.g. aluminium(III) chloride, boron trifluoride or its etherate, titanium(IV) chloride, tin(IV) chloride) and organic acids (e.g. formic acid, acetic acid, propionic acid, malonic acid, lactic acid, oxalic acid, fumaric acid, adipic acid, stearic acid, tartaric acid, oleic acid, methanesulphonic acid, benzoic acid, benzenesulphonic acid or para-toluenesulphonic acid).

Isomers Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. Accordingly, the invention encompasses both pure stereoisomers and any mixture of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. Preferably excluded herefrom are methods for the surgical or therapeutic treatment of the human or animal body and diagnostic methods carried out on the human or animal body.

The invention furthermore relates to the use of the compounds of the formula (I) as pesticides, in particular crop protection agents.

In the context of the present application, the term “pesticide” in each case also always comprises the term “crop protection agent”.

The compounds of the formula (I), having good plant tolerance, favourable homeotherm toxicity and good environmental compatibility, are suitable for protecting plants and plant organs against biotic and abiotic stressors, for increasing harvest yields, for improving the quality of the harvested material and for controlling animal pests, especially insects, arachnids, helminths, in particular nematodes, and molluscs, which are encountered in agriculture, in horticulture, in animal husbandry, in aquatic cultures, in forests, in gardens and leisure facilities, in the protection of stored products and of materials, and in the hygiene sector.

Within the context of the present patent application, the term “hygiene” is understood to mean any and all measures, procedures and practices which aim to prevent disease, in particular infectious disease, and which serve to protect the health of humans and animals and/or to protect the environment, and/or which maintain cleanliness. In accordance with the invention, this especially includes measures for cleaning, disinfection and sterilisation of, for example, textiles or hard surfaces, especially surfaces of glass, wood, concrete, porcelain, ceramics, plastic or also of metal(s), and for ensuring that these are kept free of hygiene pests and/or their excretions. Preferably excluded from the scope of the invention in this regard are surgical or therapeutic treatment procedures applicable to the human body or to the bodies of animals and diagnostic procedures which are carried out on the human body or on the bodies of animals.

The term “hygiene sector” thus covers all areas, technical fields and industrial applications in which these hygiene measures, procedures and practices are important, in relation for example to hygiene in kitchens, bakeries, airports, bathrooms, swimming pools, department stores, hotels, hospitals, stables, animal husbandries, etc.

The term “hygiene pest” is therefore understood to mean one or more animal pests whose presence in the hygiene sector is problematic, in particular for health reasons. It is therefore a primary objective to avoid or minimize the presence of hygiene pests, and/or exposure to them, in the hygiene sector. This can be achieved in particular through the application of a pesticide that can be used both to prevent infestation and to tackle an infestation which is already present. Preparations which avoid or reduce exposure to pests can also be used. Hygiene pests include, for example, the organisms mentioned below.

The term “hygiene protection” thus covers all actions to maintain and/or improve these hygiene measures, procedures and practices.

The compounds of the formula (I) can preferably be used as pesticides. They are active against normally sensitive and resistant species and against all or some stages of development. The abovementioned pests include:

pests from the phylum of the Arthropoda, in particular from the class of the Arachnida, for example Acarus spp., for example Acarus siro, Aceria kuko, Aceria sheldoni, Aculops spp., Aculus spp., for example Aculus fockeui, Aculus schlechtendali, Amblyomma spp., Amphitetranychus viennensis, Argas spp., Boophilus spp., Brevipalpus spp., for example Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., for example Eotetranychus hicoriae, Epitrimerus pyri, Eutetranychus spp., for example Eutetranychus banksi, Eriophyes spp., for example Eriophyes pyri, Glycyphagus domesticus, Halotydeus destructor, Hemitarsonemus spp., for example Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., for example Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., for example Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., for example Tarsonemus confusus, Tarsonemus pallidus, Tetranychus spp., for example Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, for example Blatta orientalis, Blattella asahinai, Blattella germanica, Leucophaea maderae, Loboptera decipiens, Neostylopyga rhombifolia, Panchlora spp., Parcoblatta spp., Periplaneta spp., for example Periplaneta americana, Periplaneta australasiae, Pycnoscelus surinamensis, Supella longipalpa;
from the order of the Coleoptera, for example Acalymma vittatum, Acanthoscelides obtectus, Adoretus spp., Aethina tumida, Agelastica alni, Agrilus spp., for example Agrilus planipennis, Agrilus coxalis, Agrilus bilineatus, Agrilus anxius, Agriotes spp., for example Agriotes linneatus, Agriotes mancus, Alphitobius diaperinus, Amphimallon solstitialis, Anobium punctatum, Anoplophora spp., for example Anoplophora glabripennis, Anthonomus spp., for example Anthonomus grandis, Anthrenus spp., Apion spp., Apogonia spp., Atomaria spp., for example Atomaria linearis, Attagenus spp., Baris caerulescens, Bruchidius obtectus, Bruchus spp., for example Bruchus pisorum, Bruchus rufimanus, Cassida spp., Cerotoma trifurcata, Ceutorrhynchus spp., for example Ceutorrhynchus assimilis, Ceutorrhynchus quadridens, Ceutorrhynchus rapae, Chaetocnema spp., for example Chaetocnema confinis, Chaetocnema denticulata, Chaetocnema ectypa, Cleonus mendicus, Conoderus spp., Cosmopolites spp., for example Cosmopolites sordidus, Costelytra zealandica, Ctenicera spp., Curculio spp., for example Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus furnissi, Dendroctonus spp., for example Dendroctonus ponderosae, Dermestes spp., Diabrotica spp., for example Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., for example Epilachna borealis, Epilachna varivestis, Epitrix spp., for example Epitrix cucumeris, Epitrix fuscula, Epitrix hirtipennis, Epitrix subcrinita, Epitrix tuberis, Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., for example Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., for example Leucoptera coffeella, Limonius ectypus, Lissorhoptrus oryzophilus, Listronotus (=Hyperodes) spp., Lixus spp., Luperodes spp., Luperomorpha xanthodera, Lyctus spp., Megacyllene spp., for example Megacyllene robiniae, Megascelis spp., Melanotus spp., for example Melanotus longulus oregonensis, Meligethes aeneus, Melolontha spp., for example Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., for example Otiorhynchus cribricollis, Otiorhynchus ligustici, Otiorhynchus ovatus, Otiorhynchus rugosostriarus, Otiorhynchus sulcatus, Oulema spp., for example Oulema melanopus, Oulema oryzae, Oxycetonia jucunda, Phaedon cochleariae, Phyllophaga spp., Phyllophaga helleri, Phyllotreta spp., for example Phyllotreta armoraciae, Phyllotreta pusilla, Phyllotreta ramosa, Phyllotreta striolata, Popillia japonica, Premnotrypes spp., Prostephanus truncatus, Psylliodes spp., for example Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Scolytus spp., for example Scolytus multistriatus, Sinoxylon perforans, Sitophilus spp., for example Sitophilus granarius, Sitophilus linearis, Sitophilus oryzae, Sitophilus zeamais, Sphenophorus spp., Stegobium paniceum, Sternechus spp., for example Sternechus paludatus, Symphyletes spp., Tanymecus spp., for example Tanymecus dilaticollis, Tanymecus indicus, Tanymecus palliatus, Tenebrio molitor, Tenebrioides mauretanicus, Tribolium spp., for example Tribolium audax, Tribolium castaneum, Tribolium confusum, Trogoderma spp., Tychius spp., Xylotrechus spp., Zabrus spp., for example Zabrus tenebrioides;
from the order of the Dermaptera, for example Anisolabis maritime, Forficula auricularia, Labidura riparia;
from the order of the Diptera, for example Aedes spp., for example Aedes aegypti, Aedes albopictus, Aedes sticticus, Aedes vexans, Agromyza spp., for example Agromyza frontella, Agromyza parvicornis, Anastrepha spp., Anopheles spp., for example Anopheles quadrimaculatus, Anopheles gambiae, Asphondylia spp., Bactrocera spp., for example Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., for example Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., for example Culex pipiens, Culex quinquefasciatus, Culicoides spp., Culiseta spp., Cuterebra spp., Dacus oleae, Dasineura spp., for example Dasineura brassicae, Delia spp., for example Delia antiqua, Delia coarctata, Delia florilega, Delia platura, Delia radicum, Dermatobia hominis, Drosophila spp., for example Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., for example Liriomyza brassicae, Liriomyza huidobrensis, Liriomyza sativae, Lucilia spp., for example Lucilia cuprina, Lutzomyia spp., Mansonia spp., Musca spp., for example Musca domestica, Musca domestica vicina, Oestrus spp., Oscinella frit, Paratanytarsus spp., Paralauterborniella subcincta, Pegomya or Pegomyia spp., for example Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., for example Rhagoletis cingulata, Rhagoletis completa, Rhagoletis fausta, Rhagoletis indifferens, Rhagoletis mendax, Rhagoletis pomonella, Sarcophaga spp., Simulium spp., for example Simulium meridionale, Stomoxys spp., Tabanus spp., Tetanops spp., Tipula spp., for example Tipula paludosa, Tipula simplex, Toxotrypana curvicauda;
from the order of the Hemiptera, for example Acizzia acaciaebaileyanae, Acizzia dodonaeae, Acizzia uncatoides, Acrida turrita, Acyrthosipon spp., for example Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., for example Amrasca bigutulla, Amrasca devastans, Anuraphis cardui, Aonidiella spp., for example Aonidiella aurantii, Aonidiella citrina, Aonidiella inornata, Aphanostigma piri, Aphis spp., for example Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., for example Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., for example Cacopsylla pyricola, Calligypona marginata, Capulinia spp., Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., for example Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., for example Dysaphis apiifolia, Dysaphis plantaginea, Dysaphis tulipae, Dysmicoccus spp., Empoasca spp., for example Empoasca abrupta, Empoasca fabae, Empoasca maligna, Empoasca solana, Empoasca stevensi, Eriosoma spp., for example Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., for example Icerya purchasi, Idiocerus spp., Idioscopus spp., Laodelphax striatellus, Lecanium spp., for example Lecanium corni (=Parthenolecanium corni), Lepidosaphes spp., for example Lepidosaphes ulmi, Lipaphis erysimi, Lopholeucaspis japonica, Lycorma delicatula, Macrosiphum spp., for example Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., for example Myzus ascalonicus, Myzus cerasi, Myzus ligustri, Myzus ornatus, Myzus persicae, Myzus nicotianae, Nasonovia ribisnigri, Neomaskellia spp., Nephotettix spp., for example Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., for example Paratrioza cockerelli, Parlatoria spp., Pemphigus spp., for example Pemphigus bursarius, Pemphigus populivenae, Peregrinus maidis, Perkinsiella spp., Phenacoccus spp., for example Phenacoccus madeirensis, Phloeomyzus passerinii, Phorodon humuli, Phylloxera spp., for example Phylloxera devastatrix, Phylloxera notabilis, Pinnaspis aspidistrae, Planococcus spp., for example Planococcus citri, Prosopidopsylla flava, Protopulvinaria pyriformis, Pseudaulacaspis pentagona, Pseudococcus spp., for example Pseudococcus calceolariae, Pseudococcus comstocki, Pseudococcus longispinus, Pseudococcus maritimus, Pseudococcus viburni, Psyllopsis spp., Psylla spp., for example Psylla buxi, Psylla mali, Psylla pyri, Pteromalus spp., Pulvinaria spp., Pyrilla spp., Quadraspidiotus spp., for example Quadraspidiotus juglansregiae, Quadraspidiotus ostreaeformis, Quadraspidiotus perniciosus, Quesada gigas, Rastrococcus spp., Rhopalosiphum spp., for example Rhopalosiphum maidis, Rhopalosiphum oxyacanthae, Rhopalosiphum padi, Rhopalosiphum rufiabdominale, Saissetia spp., for example Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., for example Toxoptera aurantii, Toxoptera citricidus, Trialeurodes vaporariorum, Trioza spp., for example Trioza diospyri, Typhlocyba spp., Unaspis spp., Viteus vitifolii, Zygina spp.;
from the suborder of the Heteroptera, for example Aelia spp., Anasa tristis, Antestiopsis spp., Boisea spp., Blissus spp., Calocoris spp., Campylomma livida, Cavelerius spp., Cimex spp., for example Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., for example Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., for example Lygocoris pabulinus, Lygus spp., for example Lygus elisus, Lygus hesperus, Lygus lineolaris, Macropes excavatus, Megacopta cribraria, Miridae, Monalonion atratum, Nezara spp., for example Nezara viridula, Nysius spp., Oebalus spp., Pentomidae, Piesma quadrata, Piezodorus spp., for example Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., for example Athalia rosae, Atta spp., Camponotus spp., Dolichovespula spp., Diprion spp., for example Diprion similis, Hoplocampa spp., for example Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., for example Sirex noctilio, Solenopsis invicta, Tapinoma spp., Technomyrmex albipes, Urocerus spp., Vespa spp., for example Vespa crabro, Wasmannia auropunctata, Xeris spp.;
from the order of the Isopoda, for example Armadillidium vulgare, Oniscus asellus, Porcellio scaber;
from the order of the Isoptera, for example Coptotermes spp., for example Coptotermes formosanus, Cornitermes cumulans, Cryptotermes spp., Incisitermes spp., Kalotermes spp., Microtermes obesi, Nasutitermes spp., Odontotermes spp., Porotermes spp., Reticulitermes spp., for example Reticulitermes flavipes, Reticulitermes hesperus;
from the order of the Lepidoptera, for example Achroia grisella, Acronicta major, Adoxophyes spp., for example Adoxophyes orana, Aedia leucomelas, Agrotis spp., for example Agrotis segetum, Agrotis ipsilon, Alabama spp., for example Alabama argillacea, Amyelois transitella, Anarsia spp., Anticarsia spp., for example Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., for example Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., for example Cydia nigricana, Cydia pomonella, Dalaca noctuides, Diaphania spp., Diparopsis spp., Diatraea saccharalis, Dioryctria spp., for example Dioryctria zimmermani, Earias spp., Ecdytolopha aurantium, Elasmopalpus lignosellus, Eldana saccharina, Ephestia spp., for example Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., for example Euproctis chrysorrhoea, Euxoa spp., Feltia spp., Galleria mellonella, Gracillaria spp., Grapholitha spp., for example Grapholita molesta, Grapholita prunivora, Hedylepta spp., Helicoverpa spp., for example Helicoverpa armigera, Helicoverpa zea, Heliothis spp., for example Heliothis virescens, Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., for example Leucoptera coffeella, Lithocolletis spp., for example Lithocolletis blancardella, Lithophane antennata, Lobesia spp., for example Lobesia botrana, Loxagrotis albicosta, Lymantria spp., for example Lymantria dispar, Lyonetia spp., for example Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., for example Ostrinia nubilalis, Panolis flammea, Parnara spp., Pectinophora spp., for example Pectinophora gossypiella, Perileucoptera spp., Phthorimaea spp., for example Phthorimaea operculella, Phyllocnistis citrella, Phyllonorycter spp., for example Phyllonorycter blancardella, Phyllonorycter crataegella, Pieris spp., for example Pieris rapae, Platynota stultana, Plodia interpunctella, Plusia spp., Plutella xylostella (=Plutella maculipennis), Podesia spp., for example Podesia syringae, Prays spp., Prodenia spp., Protoparce spp., Pseudaletia spp., for example Pseudaletia unipuncta, Pseudoplusia includens, Pyrausta nubilalis, Rachiplusia nu, Schoenobius spp., for example Schoenobius bipunctifer, Scirpophaga spp., for example Scirpophaga innotata, Scotia segetum, Sesamia spp., for example Sesamia inferens, Sparganothis spp., Spodoptera spp., for example Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., for example Trichoplusia ni, Tryporyza incertulas, Tuta absoluta, Virachola spp.;
from the order of the Orthoptera or Saltatoria, for example Acheta domesticus, Dichroplus spp., Gryllotalpa spp., for example Gryllotalpa gryllotalpa, Hieroglyphus spp., Locusta spp., for example Locusta migratoria, Melanoplus spp., for example Melanoplus devastator, Paratlanticus ussuriensis, Schistocerca gregaria;
from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example, Ceratophyllus spp., Ctenocephalides spp., for example Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Chaetanaphothrips leeuweni, Drepanothrips reuteri, Enneothrips flavens, Frankliniella spp., for example Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., for example Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., for example Scutigerella immaculata; pests from the phylum of the Mollusca, for example from the class of the Bivalvia, for example Dreissena spp.,
and also from the class of the Gastropoda, for example Arion spp., for example Arion ater rufus, Biomphalaria spp., Bulinus spp., Deroceras spp., for example Deroceras laeve, Galba spp., Lymnaea spp., Oncomelania spp., Pomacea spp., Succinea spp.;
plant pests from the phylum of the Nematoda, i.e. phytoparasitic nematodes, in particular Aglenchus spp., for example Aglenchus agricola, Anguina spp., for example Anguina tritici, Aphelenchoides spp., for example Aphelenchoides arachidis, Aphelenchoides fragariae, Belonolaimus spp., for example Belonolaimus gracilis, Belonolaimus longicaudatus, Belonolaimus nortoni, Bursaphelenchus spp., for example Bursaphelenchus cocophilus, Bursaphelenchus eremus, Bursaphelenchus xylophilus, Cacopaurus spp., for example Cacopaurus pestis, Criconemella spp., for example Criconemella curvata, Criconemella onoensis, Criconemella ornata, Criconemella rusium, Criconemella xenoplax (=Mesocriconema xenoplax), Criconemoides spp., for example Criconemoides ferniae, Criconemoides onoense, Criconemoides ornatum, Ditylenchus spp., for example Ditylenchus dipsaci, Dolichodorus spp., Globodera spp., for example Globodera pallida, Globodera rostochiensis, Helicotylenchus spp., for example Helicotylenchus dihystera, Hemicriconemoides spp., Hemicycliophora spp., Heterodera spp., for example Heterodera avenae, Heterodera glycines, Heterodera schachtii, Hirschmaniella spp., Hoplolaimus spp., Longidorus spp., for example Longidorus africanus, Meloidogyne spp., for example Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., for example Paratrichodorus minor, Paratylenchus spp., Pratylenchus spp., for example Pratylenchus penetrans, Pseudohalenchus spp., Psilenchus spp., Punctodera spp., Quinisulcius spp., Radopholus spp., for example Radopholus citrophilus, Radopholus similis, Rotylenchulus spp., Rotylenchus spp., Scutellonema spp., Subanguina spp., Trichodorus spp., for example Trichodorus obtusus, Trichodorus primitivus, Tylenchorhynchus spp., for example Tylenchorhynchus annulatus, Tylenchulus spp., for example Tylenchulus semipenetrans, Xiphinema spp., for example Xiphinema index.

The compounds of the formula (I) can optionally, at certain concentrations or application rates, also be used as herbicides, safeners, growth regulators or agents to improve plant properties, as microbicides or gametocides, for example as fungicides, antimycotics, bactericides, viricides (including agents against viroids) or as agents against MLO (mycoplasma-like organisms) and RLO (rickettsia-like organisms). If appropriate, they can also be used as intermediates or precursors for the synthesis of other active compounds.

### Formulations

The present invention further relates to formulations and use forms prepared therefrom as pesticides, for example drench, drip and spray liquors, comprising at least one compound of the formula (I). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropyl guar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemically active compounds.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are prepared in a known way, for example by mixing the compounds of the formula (I) with auxiliaries such as, for example, extenders, solvents and/or solid carriers and/or other auxiliaries such as, for example, surfactants. The formulations are prepared either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), the esters (including fats and oils) and (poly)ethers, the unsubstituted and substituted amines, amides, lactams (such as N-alkylpyrrolidones) and lactones, the sulphones and sulphoxides (such as dimethyl sulphoxide), the carbonates and the nitriles.

If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, carbonates such as propylene carbonate, butylene carbonate, diethyl carbonate or dibutyl carbonate, or nitriles such as acetonitrile or propanenitrile.

In principle, it is possible to use all suitable solvents. Examples of suitable solvents are aromatic hydrocarbons, such as xylene, toluene or alkylnaphthalenes, chlorinated aromatic or chlorinated aliphatic hydrocarbons, such as chlorobenzene, chloroethylene or methylene chloride, aliphatic hydrocarbons, such as cyclohexane, paraffins, petroleum fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, carbonates such as propylene carbonate, butylene carbonate, diethyl carbonate or dibutyl carbonate, nitriles such as acetonitrile or propanenitrile, and also water.

In principle, it is possible to use all suitable carriers. Useful carriers include especially: for example ammonium salts and ground natural minerals such as kaolins, clays, talc, chalk, quartz, attapulgite, montmorillonite or diatomaceous earth, and ground synthetic materials such as finely divided silica, alumina and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic meals, and also granules of organic material such as sawdust, paper, coconut shells, corn cobs and tobacco stalks.

Liquefied gaseous extenders or solvents can also be used. Particularly suitable extenders or carriers are those which are gaseous at ambient temperature and under atmospheric pressure, for example aerosol propellant gases, such as halohydrocarbons, and also butane, propane, nitrogen and carbon dioxide.

Examples of emulsifiers and/or foam-formers, dispersants or wetting agents with ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), isethionate derivatives, phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and when the application takes place in water.

It is possible to use colorants such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc as further auxiliaries in the formulations and the use forms derived therefrom.

Additional components may be stabilizers, such as low-temperature stabilizers, preservatives, antioxidants, light stabilizers or other agents which improve chemical and/or physical stability. Foam formers or antifoams may also be present.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids may also be present as additional auxiliaries in the formulations and the use forms derived therefrom. Further possible auxiliaries are mineral and vegetable oils.

Optionally, further auxiliaries may be present in the formulations and the use forms derived therefrom. Examples of such additives include fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Suitable penetrants in the present context are all those substances which are usually used for improving the penetration of agrochemical active compounds into plants. Penetrants are defined in this context by their ability to penetrate from the (generally aqueous) application liquor and/or from the spray coating into the cuticle of the plant and thereby increase the mobility of active compounds in the cuticle. The method described in the literature (Baur et al., 1997, Pesticide Science 51, 131-152) can be used to determine this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001 and 98% by weight of the compound of the formula (I) or, with particular preference, between 0.01% and 95% by weight of the compound of the formula (I), more preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms is usually between 0.00000001 and 95% by weight of the compound of the formula (I), preferably between 0.00001 and 1% by weight, based on the weight of the use form. The compounds are employed in a customary manner appropriate for the use forms.

Mixtures The compounds of the formula (I) may also be employed as a mixture with one or more suitable fungicides, bactericides, acaricides, molluscicides, nematicides, insecticides, microbiologicals, beneficial species, herbicides, fertilizers, bird repellents, phytotonics, sterilants, safeners, semiochemicals and/or plant growth regulators, in order thus, for example, to broaden the spectrum of action, to prolong the duration of action, to increase the rate of action, to prevent repulsion or prevent evolution of resistance. In addition, such active compound combinations may improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

Furthermore, the compounds of the formula (I) can be present in a mixture with other active compounds or semiochemicals such as attractants and/or bird repellants and/or plant activators and/or growth regulators and/or fertilizers. Likewise, the compounds of the formula (I) can be used to improve plant properties such as, for example, growth, yield and quality of the harvested material.

In a particular embodiment according to the invention, the compounds of the formula (I) are present in formulations or the use forms prepared from these formulations in a mixture with further compounds, preferably those as described below.

If one of the compounds mentioned below can occur in different tautomeric forms, these forms are also included even if not explicitly mentioned in each case. Further, all named mixing partners can, if their functional groups enable this, optionally form salts with suitable bases or acids.

### Insecticides/Acaricides/Nematicides

The active compounds identified here by their common names are known and are described, for example, in the pesticide handbook (“The Pesticide Manual” 16th Ed., British Crop Protection Council 2012) or can be found on the Internet (e.g. http://www.alanwood.net/pesticides). The classification is based on the current IRAC Mode of Action Classification Scheme at the time of filing of this patent application.

(1) Acetylcholinesterase (AChE) inhibitors, preferably carbamates selected from alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb, or organophosphates selected from acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, preferably cyclodiene-organochlorines selected from chlordane and endosulfan or phenylpyrazoles (fiproles), for example ethiprole and fipronil.
(3) Sodium channel modulators, preferably pyrethroids selected from acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans-isomer], deltamethrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans-isomer], prallethrin, pyrethrins (pyrethrum), resmethrin, silafluofen, tefluthrin, tetramethrin, tetramethrin [(1R)-isomer)], tralomethrin and transfluthrin or DDT or methoxychlor.
(4) Nicotinic acetylcholine receptor (nAChR) competitive modulators, such preferably neonicotinoids selected from acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, preferably spinosyns selected from spinetoram and spinosad.
(6) Glutamate-gated chloride channel (GluCl) allosteric modulators, preferably avermectins/milbemycins selected from abamectin, emamectin benzoate, lepimectin and milbemectin.
(7) Juvenile hormone mimics, preferably juvenile hormone analogues selected from hydroprene, kinoprene and methoprene, or fenoxycarb or pyriproxyfen.
(8) Miscellaneous non-specific (multi-site) inhibitors, preferably alkyl halides selected from methyl bromide and other alkyl halides, or chloropicrine or sulphuryl fluoride or borax or tartar emetic or methyl isocyanate generators selected from diazomet and metam.
(9) Chordotonal organ TRPV channel modulators selected from pymetrozine and pyrifluquinazone.
(10) Mite growth inhibitors selected from clofentezine, hexythiazox, diflovidazin and etoxazole.
(11) Microbial disruptors of the insect gut membrane selected from Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and B.t. plant proteins selected from Cry1Ab, Cry1Ac, Cry1Fa, Cry1A.105, Cry2Ab, Vip3A, mCry3A, Cry3Ab, Cry3Bb and Cry34Ab1/35Ab1.
(12) Inhibitors of mitochondrial ATP synthase, preferably ATP disruptors selected from diafenthiuron, or organotin compounds selected from azocyclotin, cyhexatin and fenbutatin oxide, or propargite or tetradifon.
(13) Uncouplers of oxidative phosphorylation via disruption of the proton gradient selected from chlorfenapyr, DNOC and sulfluramid.
(14) Nicotinic acetylcholine receptor channel blockers selected from bensultap, cartap hydrochloride, thiocylam and thiosultap-sodium.
(15) Inhibitors of chitin biosynthesis, type 0, selected from bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1 selected from buprofezin.
(17) Moulting disruptor (in particular for Diptera, i.e. dipterans) selected from cyromazine.
(18) Ecdysone receptor agonists selected from chromafenozide, halofenozide, methoxyfenozide and tebufenozide.
(19) Octopamine receptor agonists selected from amitraz.
(20) Mitochondrial complex III electron transport inhibitors selected from hydramethylnone, acequinocyl and fluacrypyrim.
(21) Mitochondrial complex I electron transport inhibitors, preferably METI acaricides selected from fenazaquin, fenpyroximate, pyrimidifen, pyridaben, tebufenpyrad and tolfenpyrad, or rotenone (Derris).
(22) Voltage-dependent sodium channel blockers selected from indoxacarb and metaflumizone.
(23) Inhibitors of acetyl CoA carboxylase, preferably tetronic and tetramic acid derivatives selected from spirodiclofen, spiromesifen and spirotetramat.
(24) Mitochondrial complex IV electron transport inhibitors, preferably phosphines selected from aluminium phosphide, calcium phosphide, phosphine and zinc phosphide, or cyanides selected from calcium cyanide, potassium cyanide and sodium cyanide.
(25) Mitochondrial complex II electron transport inhibitors, preferably beta-ketonitrile derivatives selected from cyenopyrafen and cyflumetofen, and carboxanilides selected from pyflubumide.
(28) Ryanodine receptor modulators, preferably diamides selected from chlorantraniliprole, cyantraniliprole and flubendiamide.
(29) Chordotonal organ Modulators (with undefined target site) selected from flonicamid.
(30) further active compounds selected from Afidopyropen, Afoxolaner, Azadirachtin, Benclothiaz, Benzoximate, Bifenazate, Broflanilide, Bromopropylate, Chinomethionat, Chloroprallethrin, Cryolite, Cyclaniliprole, Cycloxaprid, Cyhalodiamide, Dicloromezotiaz, Dicofol, epsilon-Metofluthrin, epsilon-Momfluthrin, Flometoquin, Fluazaindolizine, Fluensulfone, Flufenerim, Flufenoxystrobin, Flufiprole, Fluhexafon, Fluopyram, Fluralaner, Fluxametamide, Fufenozide, Guadipyr, Heptafluthrin, Imidaclothiz, Iprodione, kappa-Bifenthrin, kappa-Tefluthrin, Lotilaner, Meperfluthrin, Paichongding, Pyridalyl, Pyrifluquinazon, Pyriminostrobin, Spirobudiclofen, Tetramethylfluthrin, Tetraniliprole, Tetrachlorantraniliprole, Tigolaner, Tioxazafen, Thiofluoximate, Triflumezopyrim and iodomethane; furthermore preparations based on Bacillus firmus (T-1582, BioNeem, Votivo), and also the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethyl carbonate (known from EP2647626) (CAS 1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoro-propan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)-benzamide, 4-[5-(3,5-dichlorophenyl)-4,5-dihydro-5-(trifluoromethyl)-3-isoxazolyl]-2-methyl-N-(trans-1-oxido-3-thietanyl)-benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]-propanamide, (+)-N-[3-chloro-1-(3-pyridinyl)-1H-pyrazol-4-yl]-N-ethyl-3-[(3,3,3-trifluoropropyl)sulfinyl]-propanamide and (−)-N-[3-chloro-1-(3-pyridinyl)-1H-pyrazol-4-yl]-N-ethyl-3-[(3,3,3-trifluoropropyl)sulfinyl]-propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-Pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)-pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]-hydrazinecarboxamide (known from CN 101715774 A) (CAS 1232543-85-9); 3-(2,2-dichloroethenyl)-2,2-dimethyl-4-(1H-benzimidazol-2-yl)phenyl-cyclopropanecarboxylic acid ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]-indeno[1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-, 1-[N-[4-[1-[4-(1,1,2,2,2-pentafluoroethoxy)phenyl]-1H-1,2,4-triazol-3-yl]phenyl]carbamate]-a-L-mannopyranose (known from US 2014/0275503 A1) (CAS 1181213-14-8); 8-(2-cyclopropylmethoxy-4-trifluoromethyl-phenoxy)-3-(6-trifluoromethyl-pyridazin-3-yl)-3-aza-bicyclo[3.2.1]octane (CAS 1253850-56-4), (8-anti)-8-(2-cyclopropylmethoxy-4-trifluoromethyl-phenoxy)-3-(6-trifluoromethyl-pyridazin-3-yl)-3-aza-bicyclo[3.2.1]octane (CAS 933798-27-7), (8-syn)-8-(2-cyclopropylmethoxy-4-trifluoromethyl-phenoxy)-3-(6-trifluoromethyl-pyridazin-3-yl)-3-aza-bicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]-propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7), 5-(1,3-dioxan-2-yl)-4-[[4-(trifluoromethyl)phenyl]methoxy]-pyrimidine (known from WO 2013/115391 A1) (CAS 1449021-97-9), 3-(4-chloro-2,6-dimethylphenyl)-4-hydroxy-8-methoxy-1-methyl-1,8-diazaspiro[4.5]dec-3-en-2-one (known from WO 2010/066780 A1, WO 2011/151146 A1) (CAS 1229023-34-0), 3-(4-chloro-2,6-dimethylphenyl)-8-methoxy-1-methyl-1,8-diazaspiro[4.5]decane-2,4-dione (known from WO 2014/187846 A1) (CAS 1638765-58-8), 3-(4-chloro-2,6-dimethylphenyl)-8-methoxy-1-methyl-2-oxo-1,8-diazaspiro[4.5]dec-3-en-4-yl-carbonic acid ethyl ester (known from WO 2010/066780 A1, WO 2011151146 A1) (CAS 1229023-00-0), N-[1-[(6-chloro-3-pyridinyl)methyl]-2(1H)-pyridinylidene]-2,2,2-trifluoro-acetamide (known from DE 3639877 A1, WO 2012029672 A1) (CAS 1363400-41-2), [N(E)]-N-[1-[(6-chloro-3-pyridinyl)methyl]-2(1H)-pyridinylidene]-2,2,2-trifluoro-acetamide, (known from WO 2016005276 A1) (CAS 1689566-03-7), [N(Z)]-N-[1-[(6-chloro-3-pyridinyl)methyl]-2(1H)-pyridinylidene]-2,2,2-trifluoro-acetamide, (CAS 1702305-40-5), 3-endo-3-[2-propoxy-4-(trifluoromethyl)phenoxy]-9-[[5-(trifluoromethyl)-2-pyridinyl]oxy]-9-azabicyclo[3.3.1]nonane (known from WO 2011/105506 A1, WO 2016/133011 A1) (CAS 1332838-17-1).

### Fungicides

The active ingredients specified herein by their Common Name are known and described, for example, in The Pesticide Manual (16th Ed. British Crop Protection Council) or can be searched in the internet (e.g. www.alanwood.net/pesticides).

All named fungicidal mixing partners of the classes (1) to (15) can, if their functional groups enable this, optionally form salts with suitable bases or acids. All named mixing partners of the classes (1) to (15) can include tautomeric forms, where applicable.

1) Inhibitors of the ergosterol biosynthesis, for example (1.001) cyproconazole, (1.002) difenoconazole, (1.003) epoxiconazole, (1.004) fenhexamid, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazol, (1.016) prochloraz, (1.017) propiconazole, (1.018) prothioconazole, (1.019) Pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) Mefentrifluconazole, (1.082) Jpfentrifluconazole.
2) Inhibitors of the respiratory chain at complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) Isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of syn-epimeric racemate 1RS,4SR,9RS and anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) Pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034)N-(2-cyclopentyl-5-fluorobenzyl)-N-cyclopropyl-3-(difluoromethyl)-5-fluoro-1-methyl-1H-pyrazole-4-carboxamide, (2.035)N-(2-tert-butyl-5-methylbenzyl)-N-cyclopropyl-3-(difluoromethyl)-5-fluoro-1-methyl-1H-pyrazole-4-carboxamide, (2.036)N-(2-tert-butylbenzyl)-N-cyclopropyl-3-(difluoromethyl)-5-fluoro-1-methyl-1H-pyrazole-4-carboxamide, (2.037)N-(5-chloro-2-ethylbenzyl)-N-cyclopropyl-3-(difluoromethyl)-5-fluoro-1-methyl-1H-pyrazole-4-carboxamide, (2.038)N-(5-chloro-2-isopropylbenzyl)-N-cyclopropyl-3-(difluoromethyl)-5-fluoro-1-methyl-1H-pyrazole-4-carboxamide, (2.039)N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040)N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041)N-[1-(2,4-dichlorophenyl)-1-methoxypropan-2-yl]-3-(difluoromethyl)-1-methyl-1H-pyrazole-4-carboxamide, (2.042)N-[2-chloro-6-(trifluoromethyl)benzyl]-N-cyclopropyl-3-(difluoromethyl)-5-fluoro-1-methyl-1H-pyrazole-4-carboxamide, (2.043)N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044)N-[5-chloro-2-(trifluoromethyl)benzyl]-N-cyclopropyl-3-(difluoromethyl)-5-fluoro-1-methyl-1H-pyrazole-4-carboxamide, (2.045)N-cyclopropyl-3-(difluoromethyl)-5-fluoro-1-methyl-N-[5-methyl-2-(trifluoromethyl)benzyl]-1H-pyrazole-4-carboxamide, (2.046)N-cyclopropyl-3-(difluoromethyl)-5-fluoro-N-(2-fluoro-6-isopropylbenzyl)-1-methyl-1H-pyrazole-4-carboxamide, (2.047)N-cyclopropyl-3-(difluoromethyl)-5-fluoro-N-(2-isopropyl-5-methylbenzyl)-1-methyl-1H-pyrazole-4-carboxamide, (2.048)N-cyclopropyl-3-(difluoromethyl)-5-fluoro-N-(2-isopropylbenzyl)-1-methyl-1H-pyrazole-4-carbothioamide, (2.049)N-cyclopropyl-3-(difluoromethyl)-5-fluoro-N-(2-isopropylbenzyl)-1-methyl-1H-pyrazole-4-carboxamide, (2.050)N-cyclopropyl-3-(difluoromethyl)-5-fluoro-N-(5-fluoro-2-isopropylbenzyl)-1-methyl-1H-pyrazole-4-carboxamide, (2.051)N-cyclopropyl-3-(difluoromethyl)-N-(2-ethyl-4,5-dimethylbenzyl)-5-fluoro-1-methyl-1H-pyrazole-4-carboxamide, (2.052)N-cyclopropyl-3-(difluoromethyl)-N-(2-ethyl-5-fluorobenzyl)-5-fluoro-1-methyl-1H-pyrazole-4-carboxamide, (2.053)N-cyclopropyl-3-(difluoromethyl)-N-(2-ethyl-5-methylbenzyl)-5-fluoro-1-methyl-1H-pyrazole-4-carboxamide, (2.054)N-cyclopropyl-N-(2-cyclopropyl-5-fluorobenzyl)-3-(difluoromethyl)-5-fluoro-1-methyl-1H-pyrazole-4-carboxamide, (2.055)N-cyclopropyl-N-(2-cyclopropyl-5-methylbenzyl)-3-(difluoromethyl)-5-fluoro-1-methyl-1H-pyrazole-4-carboxamide, (2.056)N-cyclopropyl-N-(2-cyclopropylbenzyl)-3-(difluoromethyl)-5-fluoro-1-methyl-1H-pyrazole-4-carboxamide.
3) Inhibitors of the respiratory chain at complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadone, (3.010) fenamidone, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[i-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027)N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate.
4) Inhibitors of the mitosis and cell division, for example (4.001) carbendazim, (4.002) diethofencarb, (4.003) ethaboxam, (4.004) fluopicolide, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023)N-(2-bromo-6-fluorophenyl)-4-(2-chloro-4-fluorophenyl)-1,3-dimethyl-1H-pyrazol-5-amine, (4.024)N-(2-bromophenyl)-4-(2-chloro-4-fluorophenyl)-1,3-dimethyl-1H-pyrazol-5-amine, (4.025)N-(4-chloro-2,6-difluorophenyl)-4-(2-chloro-4-fluorophenyl)-1,3-dimethyl-1H-pyrazol-5-amine.
5) Compounds capable to have a multisite action, for example (5.001) bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodine, (5.012) folpet, (5.013) mancozeb, (5.014) maneb, (5.015) metiram, (5.016) metiram zinc, (5.017) oxine-copper, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.
6) Compounds capable to induce a host defence, for example (6.001) acibenzolar-S-methyl, (6.002) isotianil, (6.003) probenazole, (6.004) tiadinil.
7) Inhibitors of the amino acid and/or protein biosynthesis, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.
8) Inhibitors of the ATP production, for example (8.001) silthiofam.
9) Inhibitors of the cell wall synthesis, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Inhibitors of the lipid and membrane synthesis, for example (10.001) propamocarb, (10.002) propamocarb hydrochloride, (10.003) tolclofos-methyl.
11) Inhibitors of the melanin biosynthesis, for example (11.001) tricyclazole, (11.002) 2,2,2-trifluoroethyl {3-methyl-1-[(4-methylbenzoyl)amino]butan-2-yl}carbamate.
12) Inhibitors of the nucleic acid synthesis, for example (12.001) benalaxyl, (12.002) benalaxyl-M (kiralaxyl), (12.003) metalaxyl, (12.004) metalaxyl-M (mefenoxam).
13) Inhibitors of the signal transduction, for example (13.001) fludioxonil, (13.002) iprodione, (13.003) procymidone, (13.004) proquinazid, (13.005) quinoxyfen, (13.006) vinclozolin.
14) Compounds capable to act as an uncoupler, for example (14.001) fluazinam, (14.002) meptyldinocap.
15) Further compounds, for example (15.001) Abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) Oxathiapiprolin, (15.023) oxyfenthiin, (15.024) pentachlorophenol and salts, (15.025) phosphorous acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butanoic acid, (15.050) 5-amino-1,3,4-thiadiazole-2-thiol, (15.051) 5-chloro-N′-phenyl-N′-(prop-2-yn-1-yl)thiophene-2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one.

### Biological Pesticides as Mixing Components

The compounds of the formula (I) can be combined with biological pesticides.

Biological pesticides comprise in particular bacteria, fungi, yeasts, plant extracts and products formed by microorganisms, including proteins and secondary metabolites.

Biological pesticides comprise bacteria such as spore-forming bacteria, root-colonising bacteria and bacteria which act as biological insecticides, fungicides or nematicides.

Examples of such bacteria which are employed or can be used as biological pesticides are: Bacillus amyloliquefaciens, strain FZB42 (DSM 231179), or Bacillus cereus, in particular B. cereus strain CNCM 1-1562 or Bacillus firmus, strain 1-1582 (Accession number CNCM 1-1582) or Bacillus pumilus, in particular strain GB34 (Accession No. ATCC 700814) and strain QST2808 (Accession No. NRRL B-30087), or Bacillus subtilis, in particular strain GB03 (Accession No. ATCC SD-1397), or Bacillus subtilis strain QST713 (Accession No. NRRL B-21661) or Bacillus subtilis strain OST 30002 (Accession No. NRRL B-50421) Bacillus thuringiensis, in particular B. thuringiensis subspecies israelensis (serotype H-14), strain AM65-52 (Accession No. ATCC 1276), or B. thuringiensis subsp. aizawai, in particular strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are employed or can be used as biological pesticides are: Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii, (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (now: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SCi (Accession Number CBS 122089), Trichoderma harzianum, in particular T. harzianum rifai T39. (Accession Number CNCM I-952).

Examples of viruses which are employed or can be used as biological pesticides are: Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples which may be mentioned are:

Agrobacterium spp., Azorhizobium caulinodans, Azospirillum spp., Azotobacter spp., Bradyrhizobium spp., Burkholderia spp., in particular Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., in particular Rhizobium trifolii, Rhizopogon spp., Scleroderma spp., Suillus spp., Streptomyces spp.

Examples of plant extracts and products formed by microorganisms including proteins and secondary metabolites which are employed or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), Pyrethrum/Pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, in particular oilseed rape powder or mustard powder.

### Safener as Mixing Components

The compounds of the formula (I) can be combined with safeners such as, for example, benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Here, plants are to be understood to mean all plants and plant parts such as wanted and unwanted wild plants or crop plants (including naturally occurring crop plants), for example cereals (wheat, rice, triticale, barley, rye, oats), maize, soya bean, potato, sugar beet, sugar cane, tomatoes, pepper, cucumber, melon, carrot, watermelon, onion, lettuce, spinach, leek, beans, Brassica oleracea (e.g. cabbage) and other vegetable species, cotton, tobacco, oilseed rape, and also fruit plants (with the fruits apples, pears, citrus fruits and grapevines). Crop plants can be plants which can be obtained by conventional breeding and optimization methods or by biotechnological and genetic engineering methods or combinations of these methods, including the transgenic plants and including the plant varieties which can or cannot be protected by varietal property rights. Plants should be understood to mean all developmental stages, such as seeds, seedlings, young (immature) plants up to mature plants. Plant parts should be understood to mean all parts and organs of the plants above and below ground, such as shoot, leaf, flower and root, examples given being leaves, needles, stalks, stems, flowers, fruit bodies, fruits and seeds, and also tubers, roots and rhizomes. Parts of plants also include harvested plants or harvested plant parts and vegetative and generative propagation material, for example seedlings, tubers, rhizomes, cuttings and seeds.

Treatment according to the invention of the plants and plant parts with the compounds of the formula (I) is carried out directly or by allowing the compounds to act on the surroundings, environment or storage space by the customary treatment methods, for example by immersion, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. The invention is used with particular preference to treat plants of the respective commercially customary cultivars or those that are in use. Plant cultivars are to be understood as meaning plants having new properties (“traits”) and which have been obtained by conventional breeding, by mutagenesis or by recombinant DNA techniques. They can be cultivars, varieties, bio- or genotypes.

Transgenic plant, seed treatment and integration events The transgenic plants or plant cultivars (those obtained by genetic engineering) which are to be treated with preference in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products. Further and particularly emphasized examples of such properties are increased resistance of the plants against animal and microbial pests, such as against insects, arachnids, nematodes, mites, slugs and snails owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof), furthermore increased resistance of the plants against phytopathogenic fungi, bacteria and/or viruses owing, for example, to systemic acquired resistance (SAR), systemin, phytoalexins, elicitors and also resistance genes and correspondingly expressed proteins and toxins, and also increased tolerance of the plants to certain herbicidally active compounds, for example imidazolinones, sulphonylureas, glyphosate or phosphinothricin (for example the “PAT” gene). The genes which impart the desired traits in question may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails.

### Crop Protection—Types of Treatment

The treatment of the plants and plant parts with the compounds of the formula (I) is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, i.e. the compounds of the formula (I) are applied to the foliage, where treatment frequency and the application rate should be adjusted according to the level of infestation with the pest in question.

In the case of systemically active compounds, the compounds of the formula (I) also access the plants via the root system. The plants are then treated by the action of the compounds of the formula (I) on the habitat of the plant. This may be done, for example, by drenching, or by mixing into the soil or the nutrient solution, i.e. the locus of the plant (e.g. soil or hydroponic systems) is impregnated with a liquid form of the compounds of the formula (I), or by soil application, i.e. the compounds of the formula (I) according to the invention are introduced in solid form (e.g. in the form of granules) into the locus of the plants, or by drip application (often also referred to as “chemigation”), i.e. the liquid application of the compounds of the formula (I) according to the invention from surface or sub-surface driplines over a certain period of time together with varying amounts of water at defined locations in the vicinity of the plants. In the case of paddy rice crops, this can also be done by metering the compound of the formula (I) in a solid application form (for example as granules) into a flooded paddy field.

### Treatment of Seed

The control of animal pests by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is furthermore desirable to optimize the amount of active compound employed in such a way as to provide optimum protection for the seed and the germinating plant from attack by animal pests, but without damaging the plant itself by the active compound employed. In particular, methods for the treatment of seed should also take into consideration the intrinsic insecticidal or nematicidal properties of pest-resistant or -tolerant transgenic plants in order to achieve optimum protection of the seed and also the germinating plant with a minimum of pesticides being employed.

The present invention therefore in particular also relates to a method for the protection of seed and germinating plants, from attack by pests, by treating the seed with one of the compounds of the formula (I). The method according to the invention for protecting seed and germinating plants against attack by pests furthermore comprises a method where the seed is treated simultaneously in one operation or sequentially with a compound of the formula (I) and a mixing component. It also comprises a method where the seed is treated at different times with a compound of the formula (I) and a mixing component.

The invention likewise relates to the use of the compounds of the formula (I) for the treatment of seed for protecting the seed and the resulting plant from animal pests.

Furthermore, the invention relates to seed which has been treated with a compound of the formula (I) according to the invention so as to afford protection from animal pests. The invention also relates to seed which has been treated simultaneously with a compound of the formula (I) and a mixing component. The invention furthermore relates to seed which has been treated at different times with a compound of the formula (I) and a mixing component. In the case of seed which has been treated at different points in time with a compound of the formula (I) and a mixing component, the individual substances may be present on the seed in different layers. Here, the layers comprising a compound of the formula (I) and mixing components may optionally be separated by an intermediate layer. The invention also relates to seed where a compound of the formula (I) and a mixing component have been applied as component of a coating or as a further layer or further layers in addition to a coating.

Furthermore, the invention relates to seed which, after the treatment with a compound of the formula (I), is subjected to a film-coating process to prevent dust abrasion on the seed.

One of the advantages encountered with a systemically acting compound of the formula (I) is the fact that, by treating the seed, not only the seed itself but also the plants resulting therefrom are, after emergence, protected against animal pests. In this manner, the immediate treatment of the crop at the time of sowing or shortly thereafter can be dispensed with.

It has to be considered a further advantage that by treatment of the seed with a compound of the formula (I), germination and emergence of the treated seed may be enhanced.

It is likewise to be considered advantageous that compounds of the formula (I) can be used in particular also for transgenic seed.

Furthermore, compounds of the formula (I) can be employed in combination with compositions or compounds of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. In particular, this takes the form of seed of cereals (for example wheat, barley, rye, millet and oats), corn, cotton, soya beans, rice, potatoes, sunflowers, coffee, tobacco, canola, oilseed rape, beets (for example sugarbeets and fodder beets), peanuts, vegetables (for example tomatoes, cucumbers, bean, cruciferous vegetables, onions and lettuce), fruit plants, lawns and ornamental plants. The treatment of the seed of cereals (such as wheat, barley, rye and oats), maize, soya beans, cotton, canola, oilseed rape, vegetables and rice is of particular importance.

As already mentioned above, the treatment of transgenic seed with a compound of the formula (I) is also of particular importance. This takes the form of seed of plants which, as a rule, comprise at least one heterologous gene which governs the expression of a polypeptide with in particular insecticidal and/or nematicidal properties. The heterologous genes in transgenic seed can originate from microorganisms such as Bacillus, Rhizobium, Pseudomonas, Serratia, Trichoderma, Clavibacter, Glomus or Gliocladium. The present invention is particularly suitable for the treatment of transgenic seed which comprises at least one heterologous gene originating from Bacillus sp. It is particularly preferably a heterologous gene derived from Bacillus thuringiensis.

In the context of the present invention, the compound of the formula (I) is applied to the seed. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water to a certain stage of the rice embryo (‘pigeon breast stage’), stimulating the germination and a more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This must be ensured particularly in the case of active compounds which can exhibit phytotoxic effects at certain application rates.

In general, the compounds of the formula (I) are applied to the seed in a suitable formulation. Suitable formulations and processes for seed treatment are known to the person skilled in the art.

The compounds of the formula (I) can be converted to the customary seed dressing formulations, such as solutions, emulsions, suspensions, powders, foams, slurries or other coating compositions for seed, and also ULV formulations.

These formulations are prepared in a known manner, by mixing the compounds of the formula (I) with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed dressing formulations usable in accordance with the invention are all substances which promote wetting and which are conventionally used for the formulation of agrochemically active compounds. Preference is given to using alkylnaphthalenesulphonates, such as diisopropyl- or diisobutylnaphthalenesulphonates.

Useful dispersants and/or emulsifiers which may be present in the seed dressing formulations usable in accordance with the invention are all nonionic, anionic and cationic dispersants conventionally used for the formulation of active agrochemical ingredients. Preference is given to using nonionic or anionic dispersants or mixtures of nonionic or anionic dispersants. Suitable nonionic dispersants include in particular ethylene oxide/propylene oxide block polymers, alkylphenol polyglycol ethers and tristryrylphenol polyglycol ethers, and the phosphated or sulphated derivatives thereof. Suitable anionic dispersants are in particular lignosulphonates, polyacrylic acid salts and arylsulphonate/formaldehyde condensates.

Antifoams which may be present in the seed dressing formulations usable in accordance with the invention are all foam-inhibiting substances conventionally used for the formulation of active agrochemical ingredients. Preference is given to using silicone antifoams and magnesium stearate.

Preservatives which may be present in the seed dressing formulations usable in accordance with the invention are all substances usable for such purposes in agrochemical compositions. Examples include dichlorophene and benzyl alcohol hemiformal.

Secondary thickeners which may be present in the seed dressing formulations usable in accordance with the invention are all substances which can be used for such purposes in agrochemical compositions. Cellulose derivatives, acrylic acid derivatives, xanthan, modified clays and finely divided silica are preferred.

Adhesives which may be present in the seed dressing formulations usable in accordance with the invention are all customary binders usable in seed dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schadlingsbekampfungsmittel”, vol. 2, Springer Verlag, 1970, pp. 401-412).

The seed dressing formulations usable in accordance with the invention can be used to treat a wide variety of different kinds of seed either directly or after prior dilution with water. For instance, the concentrates or the preparations obtainable therefrom by dilution with water can be used to dress the seed of cereals, such as wheat, barley, rye, oats, and triticale, and also the seed of maize, rice, oilseed rape, peas, beans, cotton, sunflowers, soya beans and beets, or else a wide variety of different vegetable seed. The seed dressing formulations usable in accordance with the invention, or the dilute use forms thereof, can also be used to dress seed of transgenic plants.

For treatment of seed with the seed dressing formulations usable in accordance with the invention, or the use forms prepared therefrom by adding water, all mixing units usable customarily for the seed dressing are useful. Specifically, the procedure in the seed dressing is to place the seed into a mixer, operated batch-wise or continuously, to add the particular desired amount of seed dressing formulations, either as such or after prior dilution with water, and to mix everything until the formulation is distributed homogeneously on the seed. If appropriate, this is followed by a drying operation.

The application rate of the seed dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. in the field of veterinary medicine, the compounds of the formula (I) are active against animal parasites, in particular ectoparasites or endoparasites. The term endoparasite includes in particular helminths and protozoae, such as coccidia. Ectoparasites are typically and preferably arthropods, in particular insects or acarids.

In the field of veterinary medicine the compounds of the formula (I) are suitable, with favourable toxicity in warm blooded animals, for controlling parasites which occur in animal breeding and animal husbandry in livestock, breeding, zoo, laboratory, experimental and domestic animals. They are active against all or specific stages of development of the parasites.

Agricultural livestock include, for example, mammals, such as, sheep, goats, horses, donkeys, camels, buffaloes, rabbits, reindeers, fallow deers, and in particular cattle and pigs; or poultry, such as turkeys, ducks, geese, and in particular chickens; or fish or crustaceans, e.g. in aquaculture; or, as the case may be, insects such as bees.

Domestic animals include, for example, mammals, such as hamsters, guinea pigs, rats, mice, chinchillas, ferrets or in particular dogs, cats; cage birds; reptiles; amphibians or aquarium fish.

According to a particular embodiment, the compounds of the formula (I) are administered to mammals.

According to another particular embodiment, the compounds of the formula (I) are administered to birds, namely cage birds or in particular poultry.

By using the compounds of the formula (I) to control animal parasites, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling”, as used herein with regard to the animal health field, means that the compounds of the formula (I) are effective in reducing the incidence of the respective parasite in an animal infected with such parasites to innocuous levels. More specifically, “controlling”, as used herein, means that the compounds of the formula (I) are effective in killing the respective parasite, inhibiting its growth, or inhibiting its proliferation.

Exemplary arthropods include, without any limitation

from the order of the Anoplurida, for example, Haematopinus spp., Linognathus spp., Pediculus spp., Phtirus spp., Solenopotes spp.;
from the order of the Mallophagida and the suborders Amblycerina and Ischnocerina, for example Bovicola spp., Damalina spp., Felicola spp., Lepikentron spp., Menopon spp., Trichodectes spp., Trimenopon spp., Trinoton spp., Werneckiella spp.;
from the order of the Diptera and the suborders Nematocerina and Brachycerina, for example Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.
from the order of the Siphonapterida, for example Ceratophyllus spp.; Ctenocephalides spp., Pulex spp., Tunga spp., Xenopsylla spp.;
from the order of the Heteropterida, for example Cimex spp., Panstrongylus spp., Rhodnius spp., Triatoma spp.; as well as nuisance and hygiene pests from the order of the Blattarida.

Further, among the arthropods, the following acari may be mentioned by way of example, without any limitation:

from the subclass of the Acari (Acarina) and the order of the Metastigmata, for example, from the family of argasidae like Argas spp., Ornithodorus spp., Otobius spp., from the family of Ixodidae like Amblyomma spp., Dermacentor spp., Haemaphysalis spp., Hyalomma spp., Ixodes spp., Rhipicephalus (Boophilus) spp, Rhipicephalus spp. (the original genus of multi host ticks); from the order of mesostigmata like Dermanyssus spp., Ornithonyssus spp., Pneumonyssus spp., Raillietia spp., Sternostoma spp., Tropilaelaps spp., Varroa spp.; from the order of the Actinedida (Prostigmata), for example Acarapis spp., Cheyletiella spp., Demodex spp., Listrophorus spp., Myobia spp., Neotrombicula spp., Ornithocheyletia spp., Psorergates spp., Trombicula spp.; and from the order of the Acaridida (Astigmata), for example Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Exemplary parasitic protozoa include, without any limitation:

Mastigophora (Flagellata) such as:
Metamonada: from the order Diplomonadida, for example, Giardia spp., Spironucleus spp.
Parabasala: from the order Trichomonadida, for example, Histomonas spp., Pentatrichomonas spp., Tetratrichomonas spp., Trichomonas spp., Tritrichomonas spp.
Euglenozoa: from the order Trypanosomatida, for example, Leishmania spp., Trypanosoma spp
Sarcomastigophora (Rhizopoda), such as Entamoebidae, for example, Entamoeba spp., Centramoebidae, for example, Acanthamoeba sp., Euamoebidae, e.g. Hartmanella sp.
Alveolata such as Apicomplexa (Sporozoa): e.g. Cryptosporidium spp.; from the order Eimeriida, for example, Besnoitia spp., Cystoisospora spp., Eimeria spp., Hammondia spp., Isospora spp., Neospora spp., Sarcocystis spp., Toxoplasma spp.; from the order Adeleida e.g. Hepatozoon spp., Klossiella spp.; from the order Haemosporida e.g. Leucocytozoon spp., Plasmodium spp.; from the order Piroplasmida e.g. Babesia spp., Ciliophora spp., Echinozoon spp., Theileria spp.; from the order Vesibuliferida e.g. Balantidium spp., Buxtonella spp.
Microspora such as Encephalitozoon spp., Enterocytozoon spp., Globidium spp., Nosema spp., and furthermore, e.g. Myxozoa spp.
Helminths pathogenic for humans or animals include, for example, acanthocephala, nematodes, pentastoma and platyhelmintha (e.g. monogenea, cestodes and trematodes).

Exemplary helminths include, without any limitation:

### Monogenea: e.g.: Dactylogyrus spp., Gyrodactylus spp., Microbothrium spp., Polystoma spp., Troglocephalus spp.

Cestodes: from the order of the Pseudophyllidea, for example: Bothridium spp., Diphyllobothrium spp., Diplogonoporus spp., Ichthyobothrium spp., Ligula spp., Schistocephalus spp., Spirometra spp. from the order of the Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.
Trematodes: from the class of the Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Ornithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes: from the order of the Trichinellida, for example: Capillaria spp., Eucoleus spp., Paracapillaria spp., Trichinella spp., Trichomosoides spp., Trichuris spp.
from the order of the Tylenchida, for example: Micronema spp., Parastrongyloides spp., Strongyloides spp.
from the order of the Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Ornithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.
from the order of the Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.
Acantocephala: from the order of the Oligacanthorhynchida, for example: Macracanthorhynchus spp., Prosthenorchis spp.; from the order of the Moniliformida, for example: Moniliformis spp.
from the order of the Polymorphida, for example: Filicollis spp.; from the order of the Echinorhynchida, for example: Acanthocephalus spp., Echinorhynchus spp., Leptorhynchoides spp.
Pentastoma: from the order of the Porocephalida, for example: Linguatula spp.

In the veterinary field and in animal keeping, the administration of the compounds of the formula (I) is carried out by methods generally known in the art, such as enterally, parenterally, dermally or nasally, in the form of suitable preparations. Administration can be carried out prophylactically, methaphylactically or therapeutically.

Thus, one embodiment of the present invention refers to the compounds of the formula (I) for use as a medicament.

Another aspect refers to the compounds of the formula (I) for use as an antiendoparasitical agent.

Another particular aspect refers to the compounds of the formula (I) for use as a anthelmintic agent, more particular for use as a nematicidal agent, a platyhelminthicidal agent, an acanthocephalicidal agent, or a pentastomicidal agent.

Another particular aspect refers to the compounds of the formula (I) for use as an antiprotozoal agent.

Another aspect refers to the compounds of the formula (I) for use as an antiectoparasitical agent, in particular an arthropodicidal agent, more particular an insecticidal agent or acaricidal agent.

Further aspects of the invention are veterinary formulations, comprising an effective amount of at least one compound of the formula (I) and at least one of the following: pharmaceutically acceptable excipient (e.g. solid or liquid diluents), pharmaceutically acceptable auxiliary (e.g. surfactants), in particular a pharmaceutically acceptable excipient and/or pharmaceutically acceptable auxiliary which is normally used in veterinary formulations.

A related aspect of the invention is a method for preparing a veterinary formulation as described herein, comprising the step of mixing at least one compound of the formula (I) with pharmaceutically acceptable excipients and/or auxiliaries, in particular with pharmaceutically acceptable excipients and/or auxiliaries which are normally used in veterinary formulations.

Another particular aspect of the invention are veterinary formulations, selected from the group of ectoparasiticidal and endoparasiticidal formulations, more particular selected from the group of anthelmintic, antiprotozoal, and arthropodicidal formulations, even more particular selected from the group of nematicidal, platyhelminthicidal, acanthocephalicidal, pentastomicidal, insecticidal, and acaricidal formulations, in accordance with the mentioned aspects, as well as their methods for preparation.

Another aspect refers to a method for treatment of a parasitic infection, in particular an infection by a parasite selected from the group of ectoparasites and endoparasites mentioned herein, by applying an effective amount of a compound of the formula (I) to an animal, in particular a non-human animal, in need thereof.

Another aspect refers to a method for treatment of a parasitic infection, in particular an infection by a parasite selected from the group of ectoparasites and endoparasites mentioned herein, by applying a veterinary formulation as defined herein to an animal, in particular a non-human animal, in need thereof.

Another aspect refers to the use of the compounds of the formula (I) in the treatment of a parasitic infection, in particular an infection by a parasite selected from the group of ectoparasites and endoparasites mentioned herein, in an animal, in particular a non-human animal.

In the present context of the animal health or veterinary field, the term “treatment” includes prophylactic, metaphylactic or therapeutical treatment.

In a particular embodiment, mixtures of at least one compound of the formula (I) with other active ingredients, particularly with endo- and ectoparasiticides, for the veterinary field are provided herewith.

In the field of animal health “mixture” not only means that two (or more) different active ingredients are formulated in a joint formulation and are accordingly applied together but also refers to products which comprise separate formulations for each active compound. Accordingly, if more than two active compounds are to be applied, all active compounds may be formulated in a joint formulation or all active compounds may be formulated in separate formulations; also feasible are mixed forms where some of the active compounds are formulated jointly and some of the active compounds are formulated separately. Separate formulations allow the separate or successive application of the active compounds in question.

The active compounds specified herein by their common names are known and described, for example, in the Pesticide Manual (see above) or can be searched in the internet (e.g. http://www.alanwood.net/pesticides).

Exemplary active ingredients from the group of ectoparasiticides, as mixing partners, include, without limitation insecticides and acaricides listed in detail above. Further active ingredients which may be used are listed below following the aforementioned classification which is based on the current IRAC Mode of Action Classification Scheme: (1) Acetylcholinesterase (AChE) inhibitors; (2) GABA-gated chloride channel blockers; (3) Sodium channel modulators; (4) Nicotinic acetylcholine receptor (nAChR) competitive modulators; (5) Nicotinic acetylcholine receptor (nAChR) allosteric modulators; (6) Glutamate-gated chloride channel (GluCl) allosteric modulators; (7) Juvenile hormone mimics; (8) Miscellaneous non-specific (multi-site) inhibitors; (9) Modulators of Chordotonal Organs; (10) Mite growth inhibitors; (12) Inhibitors of mitochondrial ATP synthase, such as, ATP disruptors; (13) Uncouplers of oxidative phosphorylation via disruption of the proton gradient; (14) Nicotinic acetylcholine receptor channel blockers; (15) Inhibitors of chitin biosynthesis, type 0; (16) Inhibitors of chitin biosynthesis, type 1; (17) Moulting disruptor (in particular for Diptera, i.e. dipterans); (18) Ecdysone receptor agonists; (19) Octopamine receptor agonists; (21) Mitochondrial complex I electron transport inhibitors; (25) Mitochondrial complex II electron transport inhibitors; (20) Mitochondrial complex III electron transport inhibitors; (22) Voltage-dependent sodium channel blockers; (23) Inhibitors of acetyl CoA carboxylase; (28) Ryanodine receptor modulators; Active compounds with unknown or non-specific mode of action, e.g., fentrifanil, fenoxacrim, cycloprene, chlorobenzilate, chlordimeform, flubenzimine, dicyclanil, amidoflumet, quinomethionate, triarathene, clothiazoben, tetrasul, potassium oleate, petroleum, metoxadiazone, gossyplure, flutenzin, bromopropylate, cryolite;

Compounds from other classes, e.g. butacarb, dimetilan, cloethocarb, phosphocarb, pirimiphos (-ethyl), parathion (-ethyl), methacrifos, isopropyl o-salicylate, trichlorfon, tigolaner, sulprofos, propaphos, sebufos, pyridathion, prothoate, dichlofenthion, demeton-S-methylsulphone, isazofos, cyanofenphos, dialifos, carbophenothion, autathiofos, aromfenvinfos (-methyl), azinphos (-ethyl), chlorpyrifos (-ethyl), fosmethilan, iodofenphos, dioxabenzofos, formothion, fonofos, flupyrazofos, fensulfothion, etrimfos;

organochlorines, e.g. camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-), metofluthrin, profluthrin, flufenprox, flubrocythrinate, fubfenprox, fenfluthrin, protrifenbute, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated carbonhydrogen compounds (HCHs),
neonicotinoids, e.g. nithiazine
dicloromezotiaz, triflumezopyrim
macrocyclic lactones, e.g. nemadectin, ivermectin, latidectin, moxidectin, selamectin, eprinomectin, doramectin, emamectin benzoate; milbemycin oxime
triprene, epofenonane, diofenolan;
Biologicals, hormones or pheromones, for example natural products, e.g. thuringiensin, codlemone or neem components
dinitrophenols, e.g. dinocap, dinobuton, binapacryl;
benzoylureas, e.g. fluazuron, penfluron,
amidine derivatives, e.g. chlormebuform, cymiazole, demiditraz
Bee hive varroa acaricides, for example organic acids, e.g. formic acid, oxalic acid.

Exemplary active ingredients from the group of endoparasiticides, as mixing partners, include, without limitation, anthelmintically active compounds and antiprotozoal active compounds.

Anthelmintically active compounds, including, without limitation, the following nematicidally, trematicidally and/or cestocidally active compounds:

from the class of macrocyclic lactones, for example: eprinomectin, abamectin, nemadectin, moxidectin, doramectin, selamectin, lepimectin, latidectin, milbemectin, ivermectin, emamectin, milbemycin;
from the class of benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole-sulphoxide, albendazole, flubendazole;
from the class of depsipeptides, preferably cyclic depsipetides, in particular 24-membered cyclic depsipeptides, for example: emodepside, PF1022A;
from the class of tetrahydropyrimidines, for example: morantel, pyrantel, oxantel;
from the class of imidazothiazoles, for example: butamisole, levamisole, tetramisole;
from the class of aminophenylamidines, for example: amidantel, deacylated amidantel (dAMD), tribendimidine;
from the class of aminoacetonitriles, for example: monepantel;
from the class of paraherquamides, for example: paraherquamide, derquantel;
from the class of salicylanilides, for example: tribromsalan, bromoxanide, brotianide, clioxanide, closantel, niclosamide, oxyclozanide, rafoxanide;
from the class of substituted phenols, for example: nitroxynil, bithionol, disophenol, hexachlorophene, niclofolan, meniclopholan;
from the class of organophosphates, for example: trichlorfon, naphthalofos, dichlorvos/DDVP, crufomate, coumaphos, haloxon;
from the class of piperazinones/quinolines, for example: praziquantel, epsiprantel;
from the class of piperazines, for example: piperazine, hydroxyzine;
from the class of tetracyclines, for example: tetracyclin, chlorotetracycline, doxycyclin, oxytetracyclin, rolitetracyclin;
from diverse other classes, for example: bunamidine, niridazole, resorantel, omphalotin, oltipraz, nitroscanate, nitroxynile, oxamniquine, mirasan, miracil, lucanthone, hycanthone, hetolin, emetine, diethylcarbamazine, dichlorophen, diamfenetide, clonazepam, bephenium, amoscanate, clorsulon.

Antiprotozoal active compounds, including, without limitation, the following active compounds:

from the class of triazines, for example: diclazuril, ponazuril, letrazuril, toltrazuril;
from the class of polylether ionophore, for example: monensin, salinomycin, maduramicin, narasin;
from the class of macrocyclic lactones, for example: milbemycin, erythromycin;
from the class of quinolones, for example: enrofloxacin, pradofloxacin;
from the class of quinines, for example: chloroquine;
from the class of pyrimidines, for example: pyrimethamine;
from the class of sulfonamides, for example: sulfaquinoxaline, trimethoprim, sulfaclozin;
from the class of thiamines, for example: amprolium;
from the class of lincosamides, for example: clindamycin;
from the class of carbanilides, for example: imidocarb;
from the class of nitrofuranes, for example: nifurtimox;
from the class of quinazolinone alkaloids, for example: halofuginon;
from diverse other classes, for example: oxamniquin, paromomycin;
from the class of vaccines or antigenes from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All named mixing partners can, if their functional groups enable this, optionally form salts with suitable bases or acids.

### Vector Control

The compounds of the formula (I) can also be used in vector control. For the purpose of the present invention, a vector is an arthropod, in particular an insect or arachnid, capable of transmitting pathogens such as, for example, viruses, worms, single-cell organisms and bacteria from a reservoir (plant, animal, human, etc.) to a host. The pathogens can be transmitted either mechanically (for example trachoma by non-stinging flies) to a host, or by injection (for example malaria parasites by mosquitoes) into a host.

Examples of vectors and the diseases or pathogens they transmit are:

### 1) Mosquitoes

Anopheles: malaria, filariasis;
Culex: Japanese encephalitis, other viral diseases, filariasis, transmission of other worms;
Aedes: yellow fever, dengue fever, other viral diseases, filariasis;
Simuliidae: transmission of worms, in particular Onchocerca volvulus;
Psychodidae: transmission of leishmaniasis
   2) Lice: skin infections, epidemic typhus;
   3) Fleas: plague, endemic typhus, cestodes;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;
   6) Ticks: borellioses such as Borrelia burgdorferi sensu lato., Borrelia duttoni, tick-borne encephalitis, Q fever (Coxiella burnetii), babesioses (Babesia canis canis), ehrlichiosis.

Examples of vectors in the sense of the present invention are insects, for example aphids, flies, leafhoppers or thrips, which are capable of transmitting plant viruses to plants. Other vectors capable of transmitting plant viruses are spider mites, lice, beetles and nematodes.

Further examples of vectors in the sense of the present invention are insects and arachnids such as mosquitoes, in particular of the genera Aedes, Anopheles, for example A. gambiae, A. arabiensis, A. funestus, A. dirus (malaria) and Culex, psychodids such as Phlebotomus, Lutzomyia, lice, fleas, flies, mites and ticks capable of transmitting pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in gardens and in leisure facilities, and also in the protection of materials and stored products.

### Protection of Industrial Materials

The compounds of the formula (I) are suitable for protecting industrial materials against attack or destruction by insects, for example from the orders Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protecting wood is particularly preferred.

In a further embodiment, the compounds of the formula (I) are used together with at least one further insecticide and/or at least one fungicide.

In a further embodiment, the compounds of the formula (I) are present as a ready-to-use pesticide, i.e. they can be applied to the material in question without further modifications. Suitable further insecticides or fungicides are in particular those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. Likewise, the compounds of the formula (I), alone or in combinations with other active compounds, can be used as antifouling agents.

Control of animal pests in the hygiene sector The compounds of the formula (I) are suitable for controlling animal pests in the hygiene sector. In particular, the invention can be applied in the domestic sector, in the hygiene sector and in the protection of stored products, especially for controlling insects, arachnids, ticks and mites encountered in enclosed spaces such as dwellings, factory halls, offices, vehicle cabins, animal husbandries. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

They are used, for example, in aerosols, pressure-free spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

The Preparation and Use Examples which follow illustrate the invention without limiting it.

### PREPARATION EXAMPLES

### Synthesis Example 1.1

### N-[3-(2,6-Difluorophenyl)-1,2,4-oxadiazol-5-yl]-2-(trifluoromethyl)nicotinamide Compound I-1-1

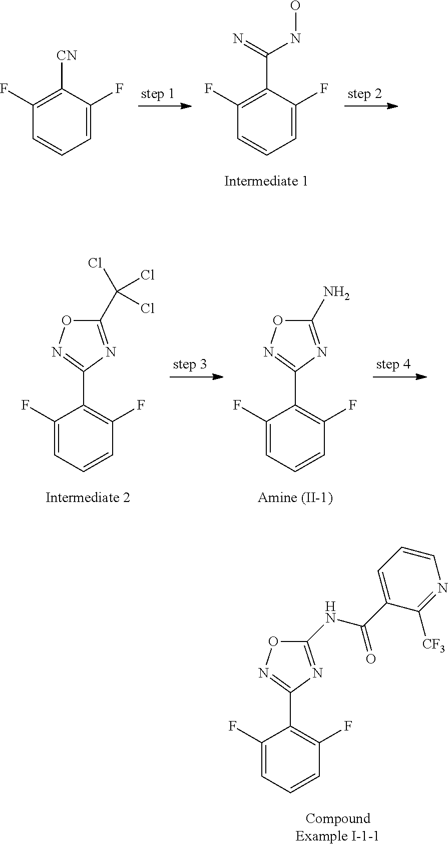 [SMILES: N#Cc1c(F)cccc1F ; N=C(NO)c1c(F)cccc1F ; Fc1cccc(F)c1-c1noc(C(Cl)(Cl)Cl)n1 ; Nc1nc(-c2c(F)cccc2F)no1 ; O=C(Nc1nc(-c2c(F)cccc2F)no1)c1cccnc1C(F)(F)F]

### Step 1: Preparation of 2,6-difluoro-N-hydroxybenzenecarboximidamide [Intermediate 1]

To a stirred solution of 2,6-difluorobenzonitrile (1.34 g) in ethanol (5 mL) was added hydroxylamine hydrochloride (2.00 g) followed by triethylamine (4.01 mL) at room temperature. The mixture was stirred overnight at 70° C. The reaction mixture was evaporated under reduced pressure. The residue obtained was diluted with water. The compound was extracted with ethyl acetate (EtOAc). After evaporation of the organic phase the title compound (0.6 g) was obtained.

GC-MS: mass (m/z): 172.

### Step 2: Preparation of 3-(2,6-difluorophenyl)-5-(trichloromethyl)-1,2,4-oxadiazole

### [Intermediate 2]:

A solution of 2,6-difluoro-N-hydroxybenzenecarboximidamide (0.6 g) in trichloroacetic anhydride (4.30 g) was heated to 120° C. After 3 hours the reaction mixture was cooled to room temperature and poured into a saturated aqueous solution of sodium hydrogenocarbonate. The aqueous layer was extracted with ethyl acetate 3 times and the combined organics were dried over sodium sulfate, filtered and concentrated. Purification by silica gel chromatography with cyclohexane/acetone afforded the title compound (0.530 g).

LC-MS: mass (m/z): 299.9 (M+H)⁺

¹H-NMR (400.0 MHz, d₆-DMSO): δ=7.81 (m, 1H), 7.42 (t, 4.8, 2H).

### Step 3: Preparation of 3-(2,6-difluorophenyl)-1,2,4-oxadiazol-5-amine [Amine (II-1)]

To 3-(2,6-difluorophenyl)-5-(trichloromethyl)-1,2,4-oxadiazole (0.530 g) was added a solution of ammonia in methanol (2.53 mL of a 7 N solution). The resulting mixture was stirred at room temperature for 5 min. The reaction mixture was then heated at 50° C. in a microwave for 1 hour. The reaction mixture was cooled down and evaporated. This afforded the title compound (0.273 g).

LC-MS: mass (m/z): 198.1 (M+H)⁺

¹H-NMR (400.0 MHz, d₆-DMSO): δ=8.08 (s br, 2H), 7.63 (m, 1H), 7.28 (t, 2H).

### Step 4: N-[3-(2,6-difluorophenyl)-1,2,4-oxadiazol-5-yl]-2-(trifluoromethyl)nicotinamide

### Compound I-1-1

To 2-(trifluoromethyl)nicotinic acid (155 mg) in dimethylformamide (1.5 mL) was added 1-[bis(dimethylamino)methylene]-1H-1,2,3-triazolo[4,5-b]pyridinium3-oxid hexafluorophosphate) (HATU) (309 mg) and diisopropylethylamine (0.265 mL). The reaction mixture was stirred at room temperature for 15 minutes. Amine (II-1) (100 mg) was added. The reaction was heated 3 hours to 80° C. The reaction mixture was cooled down. HPLC purification afforded 74.2 mg of the title compound.

HPLC-MS: mass (m/z): 371.0 (M+H)⁺

¹H-NMR (400.0 MHz, d₆-DMSO): δ=8.91 (d, 1H), 8.37 (d, 1H), 7.88 (m, 1H), 7.73 (m, 1H), 7.36 (t, 2H).

### Synthesis Example 1.2

### 2-(trifluoromethyl)-N-{3-[2-(trifluoromethyl)phenyl]-1,2,4-oxadiazol-5-yl}nicotinamide

### Compound I-1-8

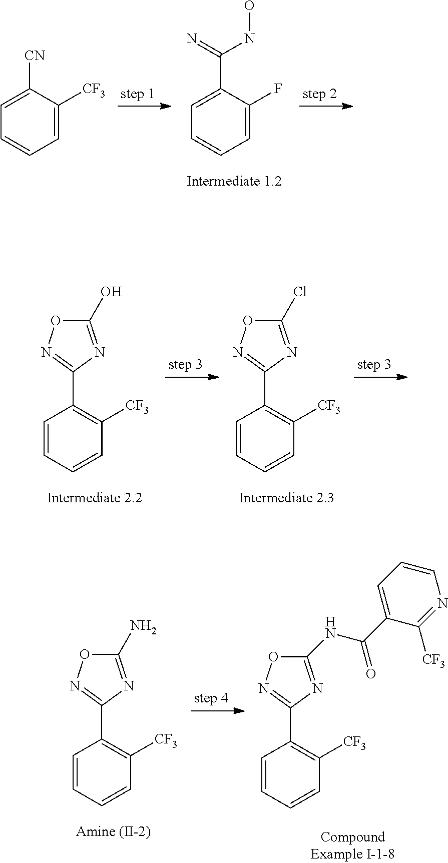 [SMILES: N#Cc1ccccc1C(F)(F)F ; N=C(NO)c1ccccc1F ; Oc1nc(-c2ccccc2C(F)(F)F)no1 ; FC(F)(F)c1ccccc1-c1noc(Cl)n1 ; Nc1nc(-c2ccccc2C(F)(F)F)no1 ; O=C(Nc1nc(-c2ccccc2C(F)(F)F)no1)c1cccnc1C(F)(F)F]

### Step 1: N′-hydroxy-2-(trifluoromethyl)benzenecarboximidamide [Intermediate 1.2]

Intermediate 1.2 was obtained analogously to Intermediate 1 (Synthesis Example 1.1) starting with 2-(trifluoromethyl)benzonitrile (25 g), methanol (190 mL), a solution of sodium hydrogen carbonate (512 mmol) and hydroxylamine hydrochloride (30.5 g).

### Step 2: 3-[2-(trifluoromethyl)phenyl]-1,2,4-oxadiazol-5-ol [Intermediate 2.2]

To a solution of Intermediate 1.2 (21.0 g) in acetonitrile, carbonyldiimidazole (23.3 g) was added and the mixture was refluxed for 4 hours. The solvent was removed in vacuo, 150 mL of water were added, and the obtained mixture was acidified to pH 3 with hydrochloric acid. The precipitate was collected by filtration and recrystallized from ethanol-water (1:1) to give 22.1 g of the title compound.

### Step 3: 5-chloro-3-[2-(trifluoromethyl)phenyl]-1,2,4-oxadiazole [Intermediate 2.3]

A mixture of Intermediate 2.2 (22.1 g), pyridine (8.50 mL), and phosphorus oxychloride (POCl₃, 100 mL) was stirred at 100° C. for 5 hours. POCl₃ was distilled off and the residue was poured onto crushed ice. The product was extracted with methyl tert-butyl ether. The organic phase was washed with an aqueous solution of sodium hydrogen carbonate and water, and evaporated under reduced pressure. The residue was extracted with hexane. The hexane solution was evaporated under reduced pressure to give 17.4 g of the title compound.

### Step 4: 3-[2-(trifluoromethyl)phenyl]-1,2,4-oxadiazol-5-amine [Amine (II-2)]

To a solution of Intermediate 2.3 (5.00 g) in dioxane (12 mL) a mixture of aqueous ammonium hydroxide (25 mL) and dioxane (25 mL) was added and the reaction mixture was stirred for 2 hours. Then, water (100 mL) was added. The precipitated solid was filtered off, dried, and recrystallized from ethanol to give 3.10 g of the title compound.

LC-MS: mass (m/z): 230.0 (M+H)⁺

¹H NMR (400 MHz, DMSO-d₆) δ 8.01 (br.s, 2H), 7.90 (d, J=8.8 Hz, 1H), 7.77 (m, 3H).

### Step 5: 2-(trifluoromethyl)-N-{3-[2-(trifluoromethyl)phenyl]-1,2,4-oxadiazol-5-yl}nicotinamide

### Compound Example I-1-8

Compound Example I-1-8 was obtained analogously to compound I-2-6 (synthesis Example 3).

¹H-NMR and HPLC-MS: see NMR Peak list table

Amine (II-3): 3-(2-chlorophenyl)-1,2,4-oxadiazol-5-amine was obtained analogously to Amine (II-2) starting with commercially available 5-chloro-3-(2-chlorophenyl)-1,2,4-oxadiazole (Intermediate 2.4).

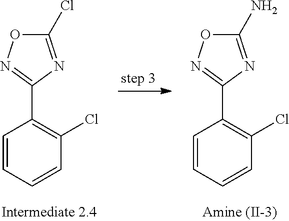 [SMILES: Clc1nc(-c2ccccc2Cl)no1 ; Nc1nc(-c2ccccc2Cl)no1]

LC-MS: mass (m/z): 196.0 (M+H)⁺

¹H NMR (400 MHz, DMSO-d₆): δ 7.97 (br.s, 2H), 7.78 (dd, J=7.6, 1.9 Hz, 1H), 7.60 (d, J=8.0 Hz, 1H), 7.53 (td, J=7.7, 1.9 Hz, 1H), 7.47 (t, J=7.5 Hz, 1H).

### Synthesis Example 1.3

### Compound Example No. I-1-12 and Compound Example No. I-1-13

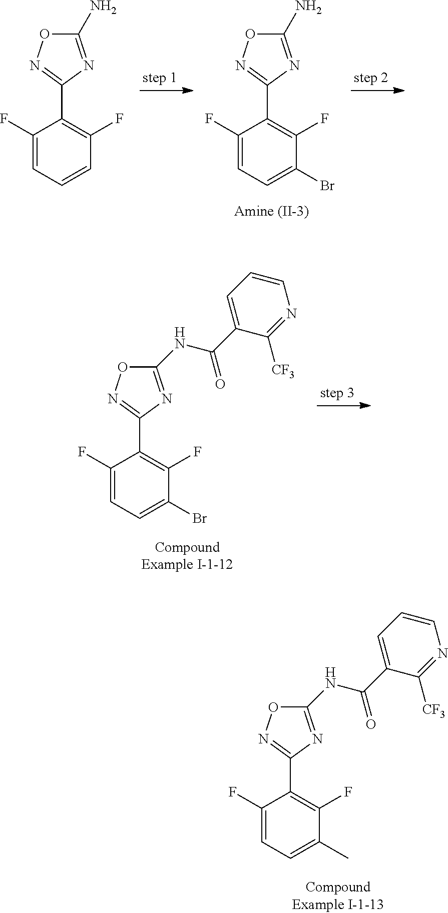 [SMILES: Nc1nc(-c2c(F)cccc2F)no1 ; Nc1nc(-c2c(F)ccc(Br)c2F)no1 ; O=C(Nc1nc(-c2c(F)ccc(Br)c2F)no1)c1cccnc1C(F)(F)F ; Cc1ccc(F)c(-c2noc(NC(=O)c3cccnc3C(F)(F)F)n2)c1F]

### Step 1: 3-(3-bromo-2,6-difluorophenyl)-1,2,4-oxadiazol-5-amine

3-(2,6-difluorophenyl)-1,2,4-oxadiazol-5-amine (100 mg), and N-bromosuccinimide (271 mg) were placed in a vial. Trifluoroacetic acid (TFA, 2 mL) was added to the vial, which was sealed. The reaction was then stirred at 60° C. for 18 h. The reaction was cooled to room temperature, diluted with 10 mL of EtOAc before neutralization of TFA with K₂CO₃ solution. The aqueous solution was extracted twice with EtOAc, dried over MgSO₄, and concentrated under vaccum. The crude yellow solid was purified by preparative HPLC to afford 68.8 mg of the title compound.

### Step 2: N-[3-(3-bromo-2,6-difluorophenyl)-1,2,4-oxadiazol-5-yl]-2-(trifluoromethyl)nicotinamide

### Compound I-1-12

Compound Example I-1-12 was obtained analogously to compound I-2-6 (Synthesis Example 3)

### Step 3: N-[3-(3-bromo-2,6-difluorophenyl)-1,2,4-oxadiazol-5-yl]-2-(trifluoromethyl)nicotinamide

### Compound I-1-13

Compound I-1-12 (90 mg), methylboronic acid (58 mg), potassium carbonate (80 mg) and Pd(dppf)Cl₂.CH₂Cl₂ (16 mg) were placed in a microwave vial in dioxane/water (2/1; 1.4 mL). Argon was added. The reaction mixture was heated at 130° C. for 30 minutes. After filtration over celite, addition of aqueous HCl, extraction with dichloromethane, and HPLC purification, 12 mg of the title compound were obtained.

### Synthesis Example 2

### N-[4-Cyano-3-(2,6-difluorophenyl)-1,2-thiazol-5-yl]-2-(trifluoromethyl)nicotinamide

### Compound I-2-1

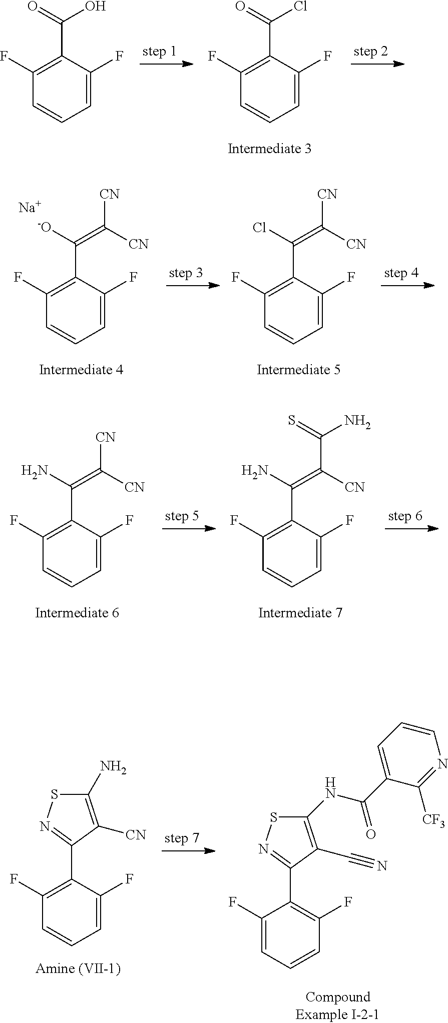

### Step 1: Preparation of Intermediate 3

A mixture of 2,6-difluoro-benzoic acid (20.0 g) and thionylchloride (75 mL) was stirred at 50° C. for 4 hours before thionylchloride was evaporated under reduced pressure. Traces of thionychloride were removed from the mixture by means of co-evaporation with benzene to afford 22.3 g of the title compound intermediate.

### Step 2: Preparation of Intermediate 4

A mixture of malonitrile (8.38 g) and sodium hydride (5.08 g) in tetrahydrofurane was stirred for 30 minutes. Then it was cooled to 0° C., Intermediate 3 (22.3 g) was added dropwise, and the mixture was stirred overnight at room temperature. The solvent was removed and water was added to the residue. The solution was extracted with dichloromethane (3×150 mL). The combined organic layer was washed with water, dried over Na₂SO₄, filtered, and evaporated in vacuo to afford 17.4 g of the title intermediate.

### Step 3: Preparation of Intermediate 5

Trimethylamine (26.0 mL) was added dropwise to a mixture of Intermediate 4 (17.0 g) and POCl₃ (11.4 g) in dichloromethane (200 mL). The reaction mixture was stirred at reflux for 15 hours. Then it was evaporated to dryness to yield 16.7 g of Intermediate 5, which was used directly in the next step.

### Step 4: Preparation of Intermediate 6

500 mL of methanolic ammonia (10%) (500 mL) were added to Intermediate 5 (16.7 g crude) at 0° C. The reaction mixture was stirred overnight at room temperature, and then evaporated to dryness under reduced pressure. Water was added to the residue and the suspension was extracted with ethyl acetate (3×100 mL). The combined organic layer was washed with water, dried over sodium sulphate and evaporated in vacuo to afford 11.3 g of the title intermediate.

### Step 5: Preparation of Intermediate 7

Triethylamine (9.21 mL) was added dropwise to a solution of Intermediate 6 (11.3 g) in pyridine (150 mL) and H₂S was bubbled through the stirring mixture for 48 hours. The solvents were evaporated under reduced pressure and the residue was dissolved in dichloromethane. The solution was washed with water (5×150 mL), dried over sodium sulphate and evaporated to afford 10.3 g of the title intermediate.

### Step 6: Preparation of 5-amino-3-(2,6-difluorophenyl)-1,2-thiazole-4-carbonitrile

### Amine (VII-1)

30% aqueous hydrogenperoxide (25 mL) was added to a solution of Intermediate 7 (5.00 g, 20.9 mmol) in methanol (50 mL) and the mixture was stirred at 50° C. for 8 hours. It was then evaporated to ¼ of the initial volume, i-PrOH was added and the mixture was left for 24 hours. Precipitated crystals were filtered to afford 3.37 g (14.2 mmol, 68%) of the title amine.

HPLC-MS: mass (m/z): 237.9 (M+H)⁺

¹H-NMR (400.0 MHz, d₆-DMSO): δ=8.3 (s br, 1H), 7.61 (m, 1H), 7.24 (t, 2H).

### Step 7: Preparation of N-[4-cyano-3-(2,6-difluorophenyl)-1,2-thiazol-5-yl]-2-(trifluoromethyl) nicotinamide

### Compound I-2-1

Compound I-2-1 was obtained analogously to Compound I-1-1 (Synthetic Example 1).

LC-MS: mass (m/z): 411.0 (M+H)⁺.

¹H-NMR (400.0 MHz, d₆-DMSO): δ=8.97 (d, 1H), 8.44 (d, 1H), 7.45 (m, 1H), 7.71 (m, 1H), 7.36 (t, 2H).

### Synthesis Example 3

2-Chloro-N-[3-(2-chlorophenyl)-4-cyano-1,2-thiazol-5-yl]nicotinamide

### Compound I-2-6

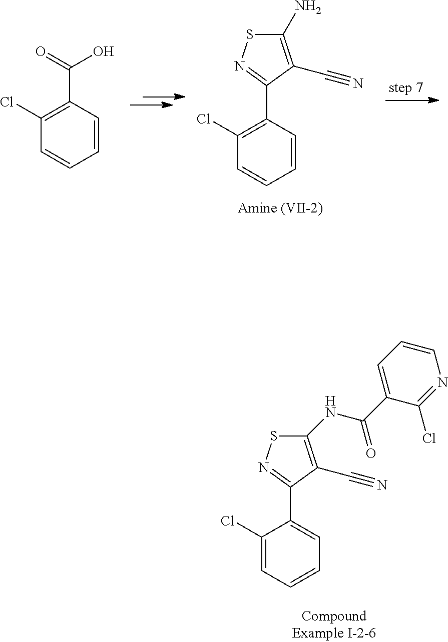 [SMILES: O=C(O)c1ccccc1Cl ; N#Cc1c(-c2ccccc2Cl)nsc1N ; N#Cc1c(-c2ccccc2Cl)nsc1NC(=O)c1cccnc1Cl]

### Preparation of 3-(3-Chloro-2,6-difluorophenyl)-1,2,4-thiadiazol-5-amine

Amine (VII-2) was obtained from 2-chlorobenzoic acid analogously to Amine (VII-1) (see Synthesis Example 2).

LC-MS: mass (m/z): 236 (M+H)⁺

¹H-NMR (400.0 MHz, d₆-DMSO): δ=8.20 (s br, 2H), 7.60 (m, 1H), 7.55 (m, 1H), 7.45 (m, 2H).

### Preparation of 2-chloro-N-[3-(2-chlorophenyl)-4-cyano-1,2-thiazol-5-yl]nicotin-amide

### Compound I-2-6

3-(3-Chloro-2,6-difluorophenyl)-1,2,4-thiadiazol-5-amine (100 mg) was placed in 4 mL of dichloromethane with N,N-diisopropylethylamine (0.081 mL). 2-Chloronicotinoyl chloride (82 mg) was added dropwise at room temperature. The reaction mixture was stirred overnight at room temperature. Water and ethylacetate were added to the reaction mixture. The aqueous layer was extracted several times with ethyl acetate, dried over sodium sulphate and evaporated. The crude product was purified by automated flash chromatography. This afforded 79 mg of the title compound.

LC-MS: mass (m/z): 376.9 (M+H)⁺

¹H-NMR (400.0 MHz, d₆-DMSO): δ=8.62 (d, 1H), 8.26 (m, 1H), 7.67-7.50 (m, 5H).

### Synthesis Example 4

### 2-Chloro-N-{4-cyano-3-[2-(trifluoromethyl)phenyl]-1,2-thiazol-5-yl}nicotinamide

### Compound I-2-7

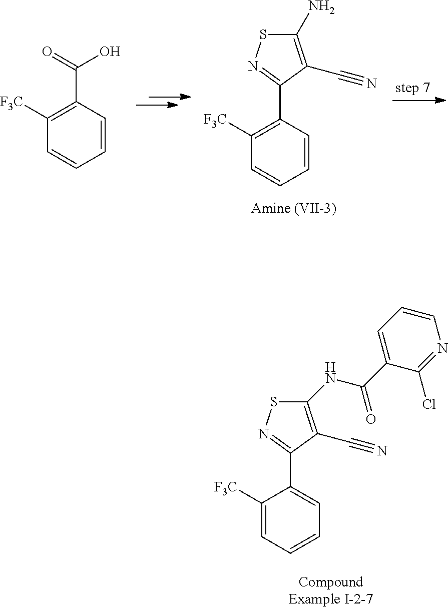 [SMILES: O=C(O)c1ccccc1C(F)(F)F ; N#Cc1c(-c2ccccc2C(F)(F)F)nsc1N ; N#Cc1c(-c2ccccc2C(F)(F)F)nsc1NC(=O)c1cccnc1Cl]

### Preparation of 5-amino-3-[2-(trifluoromethyl)phenyl]-1,2-thiazole-4-carbonitrile

Amine (VII-3) was obtained from 2-trifluoromethylbenzoic acid analogously to Amine (VII-1) (see Synthetic Example 2).

LC-MS: mass (m/z): 270.2 (M+H)⁺

¹H-NMR (400.0 MHz, d₆-DMSO): δ=8.20 (s br, 2H), 7.90 (m, 1H), 7.75 (m, 2H), 7.55 (m, 1H).

### Preparation of 2-chloro-N-{4-cyano-3-[2-(trifluoromethyl)phenyl]-1,2-thiazol-5-yl}nicotinamide

### Compound I-2-7

Compound I-2-7 was obtained analogously to Compound I-2-6.

LC-MS: mass (m/z): 409.0 (M+H)⁺

¹H-NMR (400.0 MHz, d₆-DMSO): δ=8.69-8.46 (m, 1H), 8.32-8.21 (m, 1H), 7.96-7.52 (m, 5H).

### Synthesis Example 5

### N-{5-Cyano-4-[2-(trifluoromethyl)phenyl]-1,3-thiazol-2-yl}-2-(trifluoromethyl)nicotinamide Compound I-3-2

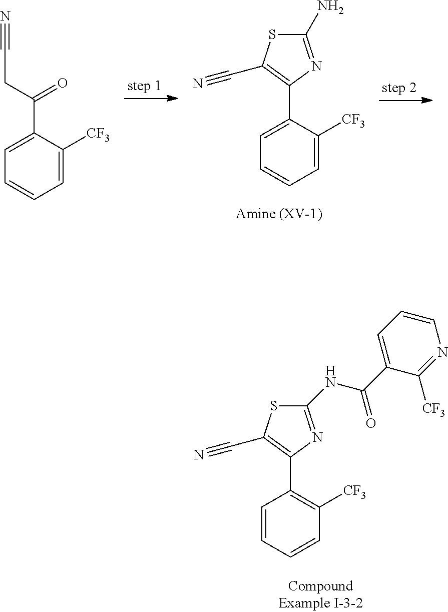 [SMILES: N#CCC(=O)c1ccccc1C(F)(F)F ; N#Cc1sc(N)nc1-c1ccccc1C(F)(F)F ; N#Cc1sc(NC(=O)c2cccnc2C(F)(F)F)nc1-c1ccccc1C(F)(F)F]

### Step 1: Preparation of Amine (XV-1):

2.50 g of 3-oxo-3-[2-(trifluoromethyl)phenyl]propanenitrile were dissolved in pyridine (15 ml) and thiourea (1.79 g) and iodine (3.01 g) were added successively. The solution was stirred at 100° C. for 12 h. The mixture was then cooled to room temperature. After work-up and preparative HPLC 650 mg of the desired amine were obtained.

LC-MS: mass (m/z): 288.0 (M+H)⁺.

Step 2: Compound I-3-2 was obtained analogously to Compound I-2-6.

LC-MS: mass (m/z): 443.0 (M+H)⁺

¹H-NMR (400.0 MHz, d₆-DMSO): δ=8.93 (d, 1H), 8.41 (d, 1H), 7.97-7.59 (m, 5H).

Compounds depicted in table 1.1, table 1.2, table 2 and table 3 were obtained analogously to Compound I-1-1 (Synthesis Example 1.1) or to Compound I-2-6 (Synthesis Example 3) using the appropriate starting carboxylic acid or starting hetaryl chloride.

TABLE 1.1
(I-1)

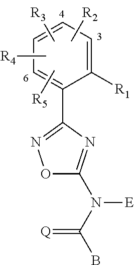

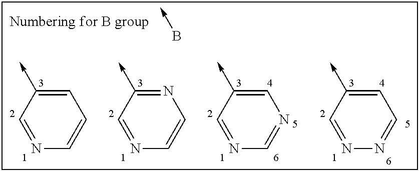

| Compound | | | | | | | | |
|---|---|---|---|---|---|---|---|---|
| Example | | | | | | | | |
| No. | B | E | R₁ | R₂ | R₃ | R₄ | R₅ | Q |
| I-1-1 | 3-(2-trifluoromethyl) | H | F | 6-F | H | H | H | O |
| Synthesis | pyridinyl | | | | | | | |
| Example | | | | | | | | |
| 1.1 | | | | | | | | |
| I-1-2 | 3-(2-difluoromethyl) | H | F | 6-F | H | H | H | O |
| | pyridinyl | | | | | | | |
| I-1-3 | 3-(2-trifluoromethyl) | H | F | 6-F | H | H | H | O |
| | pyrazinyl | | | | | | | |
| I-1-4 | 3-(2-trifluoromethyl) | H | F | 6-F | H | H | H | O |
| | pyrimidine | | | | | | | |
| I-1-5 | 3-(2-trifluoromethyl) | H | Cl | H | H | H | H | O |
| | pyridinyl | | | | | | | |
| I-1-6 | 3-(2-chloro)pyridinyl | H | Cl | H | H | H | H | O |
| I-1-7 | 3-(2-trifluoromethyl) | H | Cl | H | H | H | H | O |
| | pyrimidine | | | | | | | |
| I-1-8 | 3-(2-trifluoromethyl) | H | CF3 | H | H | H | H | O |
| Synthesis | pyridinyl | | | | | | | |
| Example | | | | | | | | |
| 1.2 | | | | | | | | |
| I-1-9 | 3-(2-chloro)pyridinyl | H | CF3 | H | H | H | H | O |
| I-1-10 | 3-(2-trifluoromethyl) | H | CF3 | H | H | H | H | O |
| | pyrimidine | | | | | | | |
| I-1-11 | 3-(2- | H | CF₃ | H | H | H | H | O |
| | trifluoromethyl)py- | | | | | | | |
| | ridazine | | | | | | | |
| I-1-12 | 3-(2-trifluoromethyl) | H | F | 3-Br | 6-F | H | H | O |
| | pyridinyl | | | | | | | |
| I-1-12 | 3-(2-trifluoromethyl) | H | F | 3-Me | 6-F | H | H | O |
| | pyridinyl | | | | | | | |
| I-1-14 | 3-(2-chloro)pyridinyl | H | F | 6-F | H | H | H | O |
| I-1-15 | 3-(2-trifluoromethyl) | H | Cl | H | H | H | H | O |
| | pyrazinyl | | | | | | | |

The determination of [M+H]⁺ or M⁻ by LC-MS under acidic chromatographic conditions was done with 1 ml formic acid per liter acetonitrile and 0.9 ml formic acid per liter Millipore water as eluents. The column Zorbax Eclipse Plus C18 50 mm*2.1 mm was used. The temperature of the column oven was 55° C.

### Instruments:

LC-MS3: Waters UPLC with SQD2 mass spectrometer and SampleManager autosampler. Linear gradient 0.0 to 1.70 minutes from 10% acetonitrile to 95% acetonitrile, from 1.70 to 2.40 minutes constant 95% acetonitrile, flow 0.85 ml/min.

LC-MS6 and LC-MS7: Agilent 1290 LC, Agilent MSD, HTS PAL autosampler. Linear gradient 0.0 to 1.80 minutes from 10% acetonitrile to 95% acetonitrile, from 1.80 to 2.50 minutes constant 95% acetonitrile, flow 1.0 ml/min.

The determination of [M+H]⁺ by LC-MS under neutral chromatographic conditions was done with acetonitrile and Millipore water containing 79 mg/l ammonia carbonate as eluents.

### Instruments:

LC-MS4: Waters IClass Acquity with QDA mass spectrometer and FTN autosampler (column Waters Acquity 1.7 μm 50 mm*2.1 mm, oven temperature 45° C.). Linear gradient 0.0 to 2.10 minutes from 10% acetonitrile to 95% acetonitrile, from 2.10 to 3.00 minutes constant 95% acetonitrile, flow 0.7 ml/min.

LC-MS5: Agilent 1100 LC system with MSD mass spectrometer and HTS PAL autosampler (column: Zorbax XDB C18 1.8 μm 50 mm*4.6 mm, oven temperature 55° C.). Linear gradient 0.0 to 4.25 minutes from 10% acetonitrile to 95% acetonitrile, from 4.25 to 5.80 minutes constant 95% acetonitrile, flow 2.0 ml/min.

Retention time indices were calculated in all cases according to a homologous series of straight chain alkan-2-ones with 3 to 16 carbons where the index of the first alkanone was set to 300, the last alkanone to 1600, the ones between correspondingly by use of a linear interpolation between successive alkanones.

The determination of the ¹H-NMR data was done with a Bruker Avance III 400 MHz spectrometer equipped with a 1.7 mm TCI probehead, with tetramethylsilane as reference (0.00 ppm) and the measurements were usually recorded from solutions in the solvents CD₃CN, CDCl₃ or d₆-DMSO. Alternatively a Bruker Avance III 600 MHz instrument equipped with a 5 mm CPNMP probehead or a Bruker Avance NEO 600 MHz instrument equipped with a 5 mm TCI probehead were used for the measurements. Usually the measurements were carried out with a probehead temperature of 298 K. Other measurement temperatures are explicitly noticed.

### NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contains therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

| Compound | LC-MS: | |
|---|---|---|
| Example | mass m/z | Peak Picking |
| I-1-2 | 353.1 | I-1-2: ¹H-NMR(400.2 MHz, d₆-DMSO): |
| | (M + H)⁺ | δ = 13.2326 (3.8); 8.8758 (5.9); 8.8726 (6.2); 8.8640 (6.2); 8.8606 (6.0); 8.3106 (5.5); |
| | | 8.2910 (5.8); 7.7804 (1.4); 7.7686 (4.5); 7.7643 (4.0); 7.7582 (5.9); 7.7485 (5.6); |
| | | 7.7428 (6.2); 7.7375 (5.5); 7.7267 (2.8); 7.7216 (3.0); 7.7054 (1.4); 7.3918 (8.8); |
| | | 7.3706 (16.0); 7.3496 (7.6); 7.3322 (3.2); 7.1978 (6.9); 7.0636 (3.4); 3.3611 (13.5); |
| | | 3.3349 (9.7); 2.6754 (1.8); 2.6710 (2.5); 2.6665 (1.8); 2.5242 (8.3); 2.5106 (158.1); |
| | | 2.5064 (305.2); 2.5020 (390.6); 2.4975 (283.6); 2.4934 (140.2); 2.3332 (1.8); 2.3288 |
| | | (2.4); 2.3243 (1.8); 2.0754 (13.1); 1.2057 (0.4); 1.1899 (0.4); 0.0080 (0.5); −0.0001 |
| | | (14.7); −0.0084 (0.5) |
| I-1-3 | 372.1 | I-1-3: ¹H-NMR(400.0 MHz, d₆-DMSO): |
| | (M + H)⁺ | δ = 13.6712 (0.4); 13.6551 (0.4); 13.5911 (1.0); 13.4985 (0.4); 9.1108 (11.5); 9.0840 |
| | | (12.2); 9.0784 (9.4); 8.3141 (2.1); 8.1355 (0.3); 7.7553 (1.5); 7.7384 (3.2); 7.7335 |
| | | (3.2); 7.7173 (6.0); 7.7010 (3.3); 7.6965 (3.6); 7.6798 (1.5); 7.3586 (8.7); 7.3371 |
| | | (16.0); 7.3157 (7.6); 6.5187 (0.5); 3.9472 (0.4); 3.4780 (0.4); 3.4021 (0.7); 3.3206 |
| | | (85.0); 2.6755 (3.6); 2.6708 (5.0); 2.6665 (3.7); 2.6621 (1.9); 2.5242 (12.2); 2.5107 |
| | | (291.1); 2.5063 (628.8); 2.5017 (890.3); 2.4972 (662.2); 2.4928 (315.1); 2.3375 (1.6); |
| | | 2.3331 (3.6); 2.3287 (4.9); 2.3241 (3.6); 2.0738 (5.7); 0.0080 (1.6); −0.0002 |
| | | (54.0); −0.0085 (1.9) |
| I-1-4 | 372.1 | I-1-4: ¹H-NMR(400.2 MHz. d₆-DMSO): |
| | (M + H)⁺ | δ = 13.5586 (0.8); 9.6301 (16.0); 9.6189 (1.1); 9.5142 (12.7); 9.4419 (0.6); 7.7754 |
| | | (1.0); 7.7591 (2.3); 7.7544 (2.3); 7.7380 (4.2); 7.7214 (2.4); 7.7169 (2.5); 7.7009 (1.0); |
| | | 7.3839 (6.1); 7.3625 (11.0); 7.3412 (5.2); 3.3475 (17.2); 2.6755 (2.2); 2.6710 (3.0); |
| | | 2.6665 (2.2); 2.5244 (10.2); 2.5107 (193.5); 2.5066 (374.2); 2.5021 (480.9); 2.4975 |
| | | (355.7); 2.4933 (183.6); 2.3333 (2.2); 2.3289 (3.0); 2.3244 (2.2); 2.0755 (11.1); 0.0079 |
| | | (0.6); −0.0002 (19.9); −0.0084 (1.0) |
| I-1-5 | 372.1 | I-1-5: ¹H-NMR(400.2 MHz, d₆-DMSO): |
| | (M + H)⁺ | δ = 13.2780 (8.2); 8.9184 (12.7); 8.9085 (12.4); 8.9065 (12.6); 8.3786 (10.3); 8.3595 |
| | | (11.1); 8.3173 (0.8); 7.9144 (10.2); 7.9024 (10.4); 7.8947 (10.2); 7.8827 (9.7); 7.8531 |
| | | (4.6); 7.8372 (4.9); 7.6888 (9.2); 7.6709 (15.8); 7.6689 (16.0); 7.6347 (7.2); 7.6305 |
| | | (7.7); 7.6162 (12.2); 7.6121 (11.9); 7.5964 (6.9); 7.5920 (6.4); 7.5573 (8.2); 7.5545 |
| | | (8.3); 7.5383 (11.8); 7.5358 (11.6); 7.5198 (4.8); 7.5171 (4.7); 3.3287 (75.0); 2.6814 |
| | | (0.9); 2.6768 (1.9); 2.6723 (2.6); 2.6677 (1.9); 2.6633 (0.9); 2.5258 (8.6); 2.5210 |
| | | (13.2); 2.5124 (167.9); 2.5079 (340.0); 2.5034 (443.4); 2.4988 (318.2); 2.4943 (154.0); |
| | | 2.3392 (0.9); 2.3346 (2.0); 2.3301 (2.7); 2.3256 (2.0); 2.3211 (1.0); 2.0760 (2.3); |
| | | 0.0080 (0.7); −0.0001 (23.6); −0.0085 (0.8) |
| I-1-6 | 369.0 | I-1-6: ¹H-NMR(400.2 MHz, d₆-DMSO): |
| | (M + H)⁺ | δ = 13.1752 (4.9); 8.5967 (11.4); 8.5920 (12.5); 8.5846 (12.3); 8.5798 (12.3); 8.3122 |
| | | (1.2); 8.2127 (11.6); 8.2080 (12.0); 8.1937 (13.0); 8.1890 (12.3); 7.8642 (7.5); 7.8481 |
| | | (8.2); 7.8451 (8.2); 7.6912 (8.2); 7.6883 (9.2); 7.6711 (15.5); 7.6683 (16.0); 7.6356 |
| | | (6.6); 7.6313 (7.5); 7.6199 (14.3); 7.6175 (14.0); 7.6129 (12.4); 7.6080 (13.1); 7.6011 |
| | | (12.4); 7.5975 (8.4); 7.5928 (7.6); 7.5890 (11.8); 7.5603 (8.2); 7.5570 (8.4); 7.5413 |
| | | (11.7); 7.5383 (11.8); 7.5227 (4.9); 7.5196 (4.7); 3.3354 (106.8); 2.6788 (0.7); 2.6743 |
| | | (1.0); 2.6697 (0.8); 2.6651 (0.4); 2.5446 (2.2); 2.5277 (3.2); 2.5229 (4.9); 2.5143 |
| | | (64.8); 2.5099 (130.6); 2.5053 (171.9); 2.5007 (125.3); 2.4962 (61.1); 2.3413 (0.4); |
| | | 2.3366 (0.8); 2.3321 (1.1); 2.3276 (0.8); 2.0755 (10.4); −0.0002 (0.6) |
| I-1-7 | 335.0 | I-1-7: ¹H-NMR(400.2 MHz, d₆-DMSO): |
| | | δ = 13.5302 (0.4); 13.4864 (0.9); 13.4208 (0.3); 9.6337 (16.0); 9.5142 (12.0); 8.3145 |
| | | (6.0); 7.9152 (0.3); 7.8971 (0.5); 7.8455 (1.4); 7.7839 (0.4); 7.6905 (3.1); 7.6715 (5.5); |
| | | 7.6395 (2.6); 7.6358 (2.8); 7.6213 (4.3); 7.6177 (4.3); 7.6014 (2.4); 7.5979 (2.2); |
| | | 7.5600 (3.0); 7.5411 (4.2); 7.5235 (1.7); 7.2046 (0.4); 7.0767 (0.3); 4.5990 (1.0); |
| | | 3.5211 (0.4); 3.5118 (0.5); 3.4879 (0.6); 3.4196 (1.6); 3.3410 (1034.7); 2.9977 (0.4); |
| | | 2.6943 (0.4); 2.6843 (2.5); 2.6797 (5.2); 2.6752 (7.1); 2.6706 (5.1); 2.6662 (2.4); |
| | | 2.6309 (0.3); 2.6139 (0.4); 2.5456 (48.6); 2.5287 (24.0); 2.5240 (37.6); 2.5153 (438.3); |
| | | 2.5109 (877.8); 2.5063 (1148.1); 2.5017 (829.2); 2.4971 (398.3); 2.3422 (2.4); 2.3377 |
| | | (5.1); 2.3331 (6.9); 2.3285 (5.1); 2.3241 (2.3); 2.0764 (6.3); 1.2143 (0.7); 1.1983 (0.7); |
| | | 1.1925 (0.4); 0.0031 (1.2) |
| I-1-8 | 403.1 | I-1-8: ¹H-NMR(400.2 MHz, d₆-DMSO): |
| | (M + H)⁺ | δ = 13.2990 (3.9); 13.2568 (0.4); 8.8852 (5.1); 8.8750 (5.2); 8.3917 (0.4); 8.3556 (3.2); |
| | | 8.3356 (3.4); 8.3161 (3.1); 7.9711 (4.8); 7.9542 (5.9); 7.8828 (4.8); 7.8634 (8.8); |
| | | 7.8451 (16.0); 7.8269 (5.7); 3.3254 (889.1); 3.2473 (0.4); 3.2056 (0.6); 3.0771 (0.4); |
| | | 3.0152 (0.5); 2.7529 (0.5); 2.6799 (3.3); 2.6754 (7.1); 2.6709 (9.7); 2.6663 (7.1); |
| | | 2.6619 (3.3); 2.6404 (0.6); 2.5243 (31.9); 2.5195 (51.9); 2.5109 (602.6); 2.5065 |
| | | (1205.6); 2.5019 (1566.7); 2.4973 (1110.8); 2.4928 (526.3); 2.4337 (1.5); 2.3377 (3.5); |
| | | 2.3334 (7.3); 2.3287 (9.9); 2.3242 (7.1); 2.3197 (3.4); 1.9886 (0.4); 1.2589 (0.4); |
| | | 1.2351 (1.0); 1.2230 (0.4); 1.1819 (1.0); 1.1659 (1.2); 1.1510 (0.6); 1.1434 (0.5); |
| | | 1.1269 (0.5); 0.1459 (8.3); 0.0079 (77.0); −0.0002 (1997.8); −0.0086 (66.9); −0.0482 |
| | | (1.0); −0.1003 (0.4); −0.1496 (8.3) |
| I-1-9 | 369.0 | I-1-9: ¹H-NMR(400.2 MHz, d₆-DMSO): |
| | (M + H)⁺ | δ = 13.2026 (5.4); 8.5874 (5.8); 8.5826 (6.4); 8.5753 (6.3); 8.5705 (6.3); 8.4583 (0.3); |
| | | 8.3721 (0.4); 8.3672 (0.4); 8.3602 (0.4); 8.3553 (0.4); 8.3164 (1.0); 8.2218 (5.4); |
| | | 8.2171 (5.7); 8.2028 (6.0); 8.1981 (5.8); 7.9852 (4.4); 7.9682 (5.4); 7.8963 (1.0); |
| | | 7.8889 (0.9); 7.8761 (4.3); 7.8642 (8.9); 7.8573 (16.0); 7.8406 (4.6); 7.8336 (3.6); |
| | | 7.8219 (1.2); 7.8178 (1.2); 7.7989 (0.5); 7.7940 (0.6); 7.7803 (0.6); 7.7754 (0.6); |
| | | 7.6118 (6.4); 7.5997 (6.3); 7.5929 (6.3); 7.5807 (6.0); 7.4390 (0.4); 7.4274 (0.4); |
| | | 7.4205 (0.4); 7.4081 (0.4); 5.7563 (0.5); 3.3242 (144.1); 2.8908 (0.4); 2.7315 (0.4); |
| | | 2.6795 (1.1); 2.6753 (2.5); 2.6708 (3.5); 2.6664 (2.6); 2.6524 (2.4); 2.5242 (10.4); |
| | | 2.5193 (15.7); 2.5107 (215.1); 2.5064 (438.9); 2.5019 (577.4); 2.4973 (418.3); 2.4929 |
| | | (205.2); 2.3332 (2.7); 2.3287 (3.6); 2.3242 (2.7); 2.0860 (0.8); 1.9886 (0.9); 1.3511 |
| | | (0.4); 1.2973 (0.4); 1.2585 (0.7); 1.2487 (0.8); 1.2333 (1.2); 1.2155 (0.4); 1.1976 (0.7); |
| | | 1.1928 (0.9); 1.1733 (3.4); 1.1562 (4.0); 1.1381 (1.8); 1.1180 (0.6); 1.0680 (0.5); |
| | | 1.0514 (0.5); 0.1457 (2.6); 0.0078 (19.8); −0.0002 (587.3); −0.0086 (21.9); −0.1498 (2.7) |
| I-1-10 | 404.1 | I-1-10: ¹H-NMR(400.2 MHz, d₆-DMSO): |
| | (M + H)⁺ | δ = 9.6132 (4.7); 9.5092 (3.3); 8.3164 (0.3); 8.1328 (0.6); 7.9767 (1.1); 7.9566 (1.4); |
| | | 7.8901 (0.3); 7.8713 (1.4); 7.8537 (4.1); 7.8372 (1.7); 7.2002 (0.4); 7.0725 (0.4); |
| | | 6.9448 (0.4); 3.3291 (46.8); 3.2064 (0.5); 3.1283 (1.1); 3.0759 (0.4); 2.6804 (0.4); |
| | | 2.6759 (0.8); 2.6714 (1.2); 2.6668 (0.8); 2.6623 (0.4); 2.5249 (3.6); 2.5202 (5.3); |
| | | 2.5114 (69.0); 2.5070 (141.7); 2.5024 (186.0); 2.4978 (131.3); 2.4933 (61.8); 2.3382 |
| | | (0.4); 2.3338 (0.8); 2.3292 (1.1); 2.3246 (0.8); 2.3201 (0.4); 2.0748 (16.0); 0.1458 |
| | | (0.7); 0.0080 (6.0); −0.0002 (187.4); −0.0086 (6.2); −0.1497 (0.7) |
| I-1-11 | 404.2 | I-1-11: ¹H-NMR(400.2 MHz, d₆-DMSO): |
| | (M + H)⁺ | δ = 20.0069 (0.6); 13.7152 (0.5); 13.6883 (0.5); 13.5728 (0.9); 9.7808 (6.1); 9.7692 |
| | | (6.4); 8.5933 (1.6); 8.3718 (5.7); 8.3617 (5.6); 8.3163 (4.4); 8.1319 (1.0); 7.9696 (4.6); |
| | | 7.9525 (6.0); 7.9093 (1.3); 7.8489 (16.0); 7.8319 (8.9); 7.7914 (2.4); 7.7701 (1.2); |
| | | 7.6725 (0.5); 7.1985 (1.7); 7.0711 (1.6); 6.9431 (1.5); 3.4332 (1.1); 3.3325 (430.6); |
| | | 3.2059 (7.8); 3.1278 (4.9); 3.0754 (6.2); 2.8909 (1.4); 2.7305 (1.2); 2.6893 (0.7); |
| | | 2.6797 (5.1); 2.6754 (11.0); 2.6708 (15.2); 2.6664 (11.0); 2.6618 (5.2); 2.5638 (2.8); |
| | | 2.5499 (5.9); 2.5244 (45.5); 2.5196 (70.0); 2.5109 (932.4); 2.5065 (1903.1); 2.5019 |
| | | (2486.4); 2.4973 (1760.8); 2.4928 (835.7); 2.4141 (1.9); 2.3379 (5.3); 2.3332 (11.2); |
| | | 2.3287 (15.4); 2.3242 (11.0); 2.3196 (5.4); 2.0745 (5.8); 1.3264 (0.5); 1.3070 (0.6); |
| | | 1.2368 (0.5); 0.1458 (10.0); 0.0323 (1.4); 0.0272 (1.6); 0.0079 (82.7); −0.0002 |
| | | (2420.7); −0.0086 (79.0); −0.1496 (10.1) |
| I-1-12 | 451.0 | I-1-12: ¹H-NMR(400.2 MHz, d₆-DMSO): |
| | (M + H)⁺ | δ = 13.4007 (8.1); 8.9219 (16.0); 8.9120 (15.8); 8.9101 (16.0); 8.8593 (0.4); 8.3948 |
| | | (13.6); 8.3758 (14.5); 8.3173 (1.3); 8.2604 (0.4); 8.2574 (0.4); 8.0958 (6.8); 8.0813 |
| | | (7.9); 8.0759 (9.1); 8.0734 (8.8); 8.0615 (9.4); 8.0590 (9.1); 8.0534 (7.7); 8.0390 (6.7); |
| | | 7.9207 (12.7); 7.9088 (12.8); 7.9010 (12.3); 7.8891 (11.7); 7.8435 (0.4); 7.8338 (0.3); |
| | | 7.8251 (0.3); 7.4238 (7.7); 7.4202 (7.8); 7.4008 (14.8); 7.3972 (14.8); 7.3778 (7.3); |
| | | 7.3742 (7.2); 4.0562 (0.7); 4.0384 (2.2); 4.0206 (2.2); 4.0028 (0.7); 3.3300 (59.3); |
| | | 2.6766 (3.4); 2.6720 (4.7); 2.6674 (3.4); 2.6628 (1.6); 2.5255 (14.1); 2.5207 (21.3); |
| | | 2.5120 (286.8); 2.5076 (579.6); 2.5031 (754.9); 2.4985 (538.5); 2.4940 (258.0); 2.3345 |
| | | (3.5); 2.3298 (4.7); 2.3254 (3.5); 1.9896 (10.2); 1.3520 (2.2); 1.2988 (2.3); 1.2591 |
| | | (3.5); 1.2332 (4.7); 1.1933 (3.0); 1.1756 (5.9); 1.1577 (2.9); 1.1012 (0.5); 1.0888 (0.4); |
| | | 1.0846 (0.5); 1.0730 (0.4); 0.8700 (0.3); 0.8534 (0.7); 0.8343 (0.4); 0.1459 (3.9); |
| | | 0.0302 (0.4); 0.0080 (30.8); −0.0001 (909.5); −0.0085 (33.3); −0.0336 (0.7); −0.1496 (4.0) |
| I-1-13 | 385.0 | I-1-13: ¹H-NMR(400.2 MHz, d₆-DMSO): |
| | (M + H)⁺ | δ = 13.3339 (2.8); 8.8997 (2.4); 8.8887 (2.4); 8.3701 (1.5); 8.3512 (1.5); 8.3164 (3.0); |
| | | 7.9523 (1.0); 7.8991 (1.6); 7.8891 (1.8); 7.8809 (1.9); 7.8690 (1.5); 7.6249 (1.0); |
| | | 7.6029 (2.1); 7.5862 (2.2); 7.5655 (1.1); 7.2676 (1.7); 7.2467 (3.2); 7.2229 (1.6); |
| | | 7.1996 (0.4); 7.0704 (0.4); 3.3789 (0.4); 3.3499 (1.0); 3.3234 (298.2); 2.8907 (7.6); |
| | | 2.7498 (0.4); 2.7308 (6.4); 2.6796 (3.8); 2.6752 (7.8); 2.6706 (10.6); 2.6660 (7.6); |
| | | 2.6614 (3.6); 2.5241 (35.9); 2.5193 (53.6); 2.5107 (626.6); 2.5062 (1246.7); 2.5016 |
| | | (1624.3); 2.4970 (1167.3); 2.4925 (553.9); 2.3375 (3.4); 2.3330 (7.4); 2.3285 (10.2); |
| | | 2.3239 (7.2); 2.3194 (3.2); 2.2842 (16.0); 2.0744 (0.4); 1.9084 (0.7); 1.4667 (0.8); |
| | | 1.4495 (0.8); 1.4176 (0.7); 1.4008 (0.7); 1.2373 (0.4); 1.0950 (0.7); 1.0780 (0.7); |
| | | 1.0735 (0.6); 1.0571 (0.6); 0.1461 (0.6); 0.0081 (4.3); −0.0002 (138.9); −0.0085 |
| | | (4.3); −0.1496 (0.5) |
| I-1-14 | 337.1 | I-1-14: ¹H-NMR(400.0 MHz, d₆-DMSO): |
| | (M + H)⁺ | δ = 13.2462 (4.5); 8.5922 (6.1); 8.5874 (6.7); 8.5801 (6.7); 8.5753 (6.5); 8.3140 (0.9); |
| | | 8.2172 (6.0); 8.2124 (6.2); 8.1982 (6.7); 8.1934 (6.3); 7.7730 (1.3); 7.7568 (2.8); |
| | | 7.7517 (2.6); 7.7405 (1.8); 7.7355 (5.3); 7.7307 (1.8); 7.7192 (2.7); 7.7142 (3.1); |
| | | 7.6980 (1.4); 7.6168 (6.6); 7.6047 (6.5); 7.5979 (6.4); 7.5857 (6.2); 7.3808 (9.0); |
| | | 7.3595 (16.0); 7.3385 (7.4); 3.3194 (107.5); 2.6751 (1.5); 2.6709 (2.2); 2.6665 (1.6); |
| | | 2.6623 (0.9); 2.6521 (0.4); 2.5243 (5.2); 2.5193 (7.9); 2.5107 (123.8); 2.5063 (267.0); |
| | | 2.5018 (378.0); 2.4973 (281.2); 2.4929 (134.2); 2.3332 (1.5); 2.3288 (2.0); 2.3241 |
| | | (1.5); 2.0739 (1.2); 1.1742 (0.4); 1.1570 (0.5); 0.0079 (0.8); −0.0002 (25.6); −0.0084 (1.0) |
| I-1-15 | 370.0 | I-1-15: ¹H-NMR(400.2 MHz, d₆-DMSO): |
| | (M + H)⁺ | δ = 13.5143 (0.8); 9.1280 (7.0); 9.1034 (8.1); 9.0980 (5.9); 8.3144 (4.6); 7.8045 (1.3); |
| | | 7.6740 (2.8); 7.6552 (5.4); 7.6275 (2.6); 7.6238 (2.6); 7.6090 (4.3); 7.6053 (4.1); |
| | | 7.5891 (2.2); 7.5852 (1.9); 7.5436 (2.7); 7.5258 (3.9); 7.5073 (1.6); 3.5095 (0.4); |
| | | 3.3344 (473.8); 3.2082 (0.4); 2.6842 (1.8); 2.6795 (3.6); 2.6750 (4.9); 2.6706 (3.5); |
| | | 2.6662 (1.7); 2.6026 (0.4); 2.5454 (5.2); 2.5284 (18.0); 2.5150 (308.4); 2.5106 (602.5); |
| | | 2.5061 (780.8); 2.5015 (564.6); 2.4971 (272.9); 2.3374 (3.5); 2.3330 (4.8); 2.3284 |
| | | (3.4); 2.3239 (1.6); 2.0762 (16.0); 0.0027 (0.7) |

TABLE 1.2
(I-2)

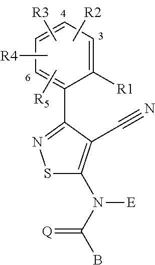

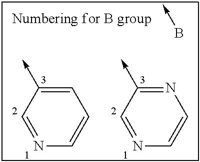

| Compound | | | | | | | | |
|---|---|---|---|---|---|---|---|---|
| Example No. | B | E | R₁ | R₂ | R₃ | R₄ | R₅ | Q |
| I-2-1 | 3-(2-trifluoromethyl) | H | F | 6-F | H | H | H | O |
| Synthesis | pyridinyl | | | | | | | |
| Example 2 | | | | | | | | |
| I-2-2 | 3-[2-(2,2,2- | H | F | 6-F | H | H | H | O |
| | trifluoroethyl)]pyridinyl | | | | | | | |
| I-2-3 | 3-[2-(pentafluoroethyl)] | H | F | 6-F | H | H | H | O |
| | pyridinyl | | | | | | | |
| I-2-4 | 3-(2-difluoromethyl) | H | F | 6-F | H | H | H | O |
| | pyridinyl | | | | | | | |
| I-2-5 | 3-(2-chloro)pyridinyl | H | F | 6-F | H | H | H | O |
| I-2-6 | 3-(2-chloro)pyridinyl | H | Cl | H | H | H | H | O |
| I-2-7 | 3-(2-chloro)pyridinyl | H | CF₃ | H | H | H | H | O |
| I-2-8 | 3-(2-trifluoromethyl) | H | CF₃ | H | H | H | H | O |
| | pyridinyl | | | | | | | |
| I-2-9 | 3-(2-trifluoromethyl) | H | Cl | H | H | H | H | O |
| | pyridinyl | | | | | | | |
| I-2-10 | 3-(2-difluoromethyl) | H | Cl | H | H | H | H | O |
| | pyridinyl | | | | | | | |
| I-2-11 | 3-(2-difluoromethyl) | H | CF₃ | H | H | H | H | O |
| | pyridinyl | | | | | | | |
| I-2-12 | 3-(2-ethyl)pyridinyl | H | F | 6-F | H | H | H | O |
| I-2-13 | 3-(2-trifluoromethyl) | H | F | 6-F | H | H | H | O |
| | pyrazinyl | | | | | | | |
| I-2-14 | 3-(2-trifluoromethyl) | H | CF₃ | H | H | H | H | O |
| | pyrazinyl | | | | | | | |
| I-2-15 | 3-(2-trifluoromethyl) | H | Cl | H | H | H | H | O |
| | pyrazinyl | | | | | | | |

Compound Example I-2-1:
LC-MS: mass (m/z): 411.0 (M + H)⁺
¹H-NMR (400.0 MHz, d₆-DMSO): δ = 8.97 (d, 1H), 8.44 (d, 1H), 7.45 (m, 1H), 7.71 (m, 1H), 7.36 (t, 2H)
Compound Example I-2-2:
LC-MS: mass (m/z): 425.0 (M + H)⁺
¹H-NMR (400.0 MHz, d₆-DMSO): δ = 8.80 (d, 1H), 8.30 (m, 1H), 7.69 (m, 1H), 7.64 (m, 1H), 7.35 (t,
2H), 4.12 (m, 2H)
Compound Example I-2-3:
LC-MS: mass (m/z): 461.0 (M + H)⁺
¹H-NMR (400.0 MHz, d₆-DMSO): ¹H-NMR (400.0 MHz, d₆-DMSO): δ = 8.97 (m, 1H), 8.41 (m, 1H), 7.94
(m, 1H), 7.69 (m, 1H), 7.35 (t, 2H)
Compound Example I-2-4:
LC-MS: mass (m/z): 393.0 (M + H)⁺
1H-NMR (400.0 MHz, d₆-DMSO): δ = 8.89 (d, 1H), 8.39 (d, 1H), 7.79 (m, 1H), 7.07 (m, 1H), 7.35 (m, 3H)
Compound Example I-2-5:
LC-MS: mass (m/z): 477.0 (M + H)⁺
¹H-NMR (400.0 MHz, d₆-DMSO): δ = 8.62 (d, 1H), 8.26 (m, 1H), 7.70 (m, 1H), 7.65 (m, 1H), 7.35 (t, 2H)
Compound Example I-2-6:
LC-MS: mass (m/z): 376.9 (M + H)⁺
¹H-NMR (400.0 MHz, d₆-DMSO): δ = 8.62 (d, 1H), 8.26 (m, 1H), 7.67-7.50 (m, 5H)
Compound Example I-2-7:
LC-MS: mass (m/z): 409.0 (M + H)⁺
¹H-NMR (400.0 MHz, d₆-DMSO): δ = 8.69-8.46 (m, 1H), 8.32-8.21 (m, 1H), 7.96-7.52 (m, 5H)
Compound Example I-2-8:
LC-MS: mass (m/z): 443.0 (M + H)⁺
¹H-NMR (400.0 MHz, d₆-DMSO): δ = 8.96 (d, 1H), 8.45 (d, 1H), 7.96 (m, 2H), 7.85-7.77 (m, 2H), 7.68
(d, 1H)
Compound Example I-2-9:
LC-MS: mass (m/z): 409.0 (M + H)⁺
¹H-NMR (400.0 MHz, d₆-DMSO): δ = 8.95 (d, 1H), 8.44 (d, 1H), 7.94 (m, 1H), 7.66 (m, 1H), 7.59 (m,
1H), 7.52 (m, 1H)
Compound Example I-2-10:
LC-MS: mass (m/z): 391.0 (M + H)⁺
¹H-NMR (400.0 MHz, d₆-DMSO): δ = 8.87 (m, 1H), 8.35 (m, 1H), 7.80-7.05 (m, 5H)
Compound Example I-2-11:
LC-MS: mass (m/z): 425.0 (M + H)⁺
¹H-NMR (400.0 MHz, d₆-DMSO): δ = 8.90 (m, 1H), 8.70 (m, 1H), 7.96-7.49 (m, 6H)
Compound Example I-2-12:
LC-MS: mass (m/z): 371.1 (M + H)⁺
¹H-NMR (400.0 MHz, d₆-DMSO): δ = 8.71 (d, 1H), 8.13 (m, 1H), 7.77 (m, 1H), 7.44 (m, 1H), 7.35 (t,
2H), 2.89 (q, 2H), 1.34 (t, 3H)
Compound Example I-2-13:
LC-MS: mass (m/z): 412.0 (M + H)⁺
¹H-NMR (400.0 MHz, d₆-DMSO): δ = 9.19 (d, 2H), 7.70 (m, 1H), 7.38 (t, 2H)
Compound Example I-2-14:
LC-MS: mass (m/z): 444.0 (M + H)⁺
¹H-NMR (400.0 MHz, d₆-DMSO): δ = 9.19 (d, 2H), 7.96 (m, 1H), 7.86 (m, 1H), 7.77 (m, 1H), 7.29 (m,
1H)
Compound Example I-2-15:
LC-MS: mass (m/z): 410.0 (M + H)⁺
¹H-NMR (400.0 MHz, d₆-DMSO): δ = 9.18 (d, 2H), 7.67-7.50 (m, 4H)

TABLE 2
(I-3)

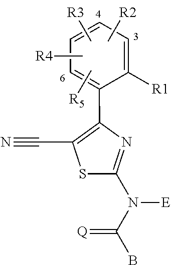

| Compound | | | | | | | | |
|---|---|---|---|---|---|---|---|---|
| Example No. | B | E | R₁ | R₂ | R₃ | R₄ | R₅ | Q |
| I-3-1 | 3-(2-trifluoromethyl) | H | F | 6-F | H | H | H | O |
| | pyridinyl | | | | | | | |
| I-3-2 | 3-(2-trifluoromethyl) | H | CF₃ | H | H | H | H | O |
| | pyridinyl | | | | | | | |
| I-3-3 | 3-(2-trifluroomethyl) | H | CF₃ | H | H | H | H | O |
| | pyrazinyl | | | | | | | |

Compound Example I-3-1:
LC-MS: mass (m/z): 411.1 (M + H)⁺
¹H-NMR (400.0 MHz, d₆-DMSO): δ = 8.94 (d, 1H), 8.41 (d, 1H), 7.92 (m, 1H), 7.70 (m, 1H), 7.37 (t,
2H)
Compound Example I-3-2:
LC-MS: mass (m/z): 443.0 (M + H)⁺
¹H-NMR (400.0 MHz, d₆-DMSO): δ = 8.93 (d, 1H), 8.41 (d, 1H), 7.97-7.59 (m, 5H)
Compound Example I-3-3:
LC-MS: mass (m/z): 444.0 (M + H)⁺
¹H-NMR (400.0 MHz, d₆-DMSO): δ = 9.16 (dd, 1H), 7.97-7.45 (m, 5H)

TABLE 3
(I-5)

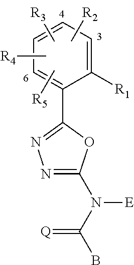

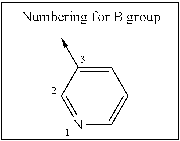

| Compound | | | | | | | | |
|---|---|---|---|---|---|---|---|---|
| Example No. | B | E | R₁ | R₂ | R₃ | R₄ | R₅ | Q |
| I-5-1 | 3-(2-difluoromethyl) | H | Cl | H | H | H | H | O |
| | pyridinyl | | | | | | | |
| I-5-2 | 3-(2-ethyl)pyridinyl | H | Cl | H | H | H | H | O |
| I-5-3 | 3-(2-difluoromethyl) | H | F | 6-F | H | H | H | O |
| | pyridinyl | | | | | | | |

| Compound | LC-MS: | |
|---|---|---|
| Example | mass m/z | Peak List |
| I-5-1 | 369.0 | I-6-1: ¹H-NMR (400.2 MHz, d₆-DMSO): |
| | (M + H)⁺ | δ = 12.8417 (11.2); 8.9119 (12.1); 8.9008 (12.2); 8.3438 (9.1); 8.3244 (10.0); 8.3148 (5.5); |
| | | 8.0081 (0.4); 7.9381 (5.2); 7.9234 (5.6); 7.9048 (9.6); 7.8928 (9.4); 7.8850 (8.8); 7.8730 |
| | | (7.4); 7.7319 (9.4); 7.7122 (16.0); 7.6738 (7.3); 7.6697 (7.6); 7.6552 (12.6); 7.6513 (12.4); |
| | | 7.6355 (7.2); 7.6312 (6.6); 7.6023 (9.0); 7.6001 (9.0); 7.5832 (12.6); 7.5646 (5.1); 3.5239 |
| | | (0.3); 3.4983 (0.3); 3.4495 (0.4); 3.3209 (691.7); 3.2603 (0.4); 2.9351 (0.3); 2.8911 (1.0); |
| | | 2.8643 (0.3); 2.8068 (0.5); 2.7782 (0.4); 2.7320 (1.1); 2.6753 (8.7); 2.6707 (11.8); 2.6662 |
| | | (8.7); 2.6619 (4.5); 2.5242 (41.6); 2.5192 (66.7); 2.5107 (689.4); 2.5063 (1342.7); 2.5018 |
| | | (1742.0); 2.4972 (1281.1); 2.4927 (636.8); 2.3375 (3.5); 2.3332 (7.8); 2.3286 (10.8); 2.3241 |
| | | (7.8); 2.3197 (3.6); 2.0740 (5.3); 1.5072 (0.3); 1.5047 (0.4); 1.4315 (0.4); 1.4237 (0.4); |
| | | 1.3411 (0.4); 1.3115 (0.3); 1.2957 (0.3); 1.2815 (0.3); 1.2693 (0.4); 1.2565 (0.4); 1.2349 |
| | | (0.7); 1.2058 (0.3): 1.1823 (0.4); 1.1652 (0.4); 0.1460 (3.0); 0.0305 (0.4); 0.0080 (27.2); −0.0002 |
| | | (738.4); −0.0085 (28.3); −0.1496 (2.9) |
| I-5-2 | 329.1 | I-6-2: ¹H-NMR (400.2 MHz, d₆-DMSO): |
| | (M + H)⁺ | δ = 12.4732 (2.6); 8.6618 (1.9); 8.6576 (2.1); 8.6497 (2.1); 8.6455 (2.1); 7.9860 (1.4); 7.9699 |
| | | (1.5); 7.9669 (1.5); 7.9502 (2.0); 7.9460 (2.1); 7.9311 (2.3); 7.9267 (2.3); 7.7371 (1.8); |
| | | 7.7342 (2.0); 7.7170 (3.2); 7.7141 (3.3); 7.6735 (1.5); 7.6692 (1.7); 7.6551 (2.6); 7.6507 |
| | | (2.5); 7.6352 (1.7); 7.6306 (1.6); 7.6062 (2.1); 7.6027 (2.2); 7.5871 (2.8); 7.5838 (2.8); |
| | | 7.5685 (1.2); 7.5652 (1.2); 7.3919 (2.0); 7.3798 (2.0); 7.3726 (2.0); 7.3604 (1.9); 5.7559 |
| | | (2.1); 3.3251 (26.8); 2.9348 (1.0); 2.9162 (3.0); 2.8974 (3.0); 2.8787 (1.0); 2.6759 (0.4); |
| | | 2.6712 (0.6); 2.6667 (0.4); 2.5248 (2.0); 2.5201 (3.1); 2.5114 (34.8); 2.5069 (70.0); 2.5023 |
| | | (92.2); 2.4977 (67.5); 2.4931 (33.2); 2.3337 (0.4); 2.3291 (0.6); 2.3246 (0.4); 1.2658 (0.4); |
| | | 1.2554 (7.2); 1.2367 (16.0); 1.2179 (7.0); −0.0002 (4.0) |
| I-5-3 | 371.0 | I-6-3: ¹H-NMR (400.2 MHz, d₆-DMSO): |
| | (M + H)⁺ | δ = 12.8926 (5.7); 8.9103 (7.6); 8.8994 (7.6); 8.3528 (5.4); 8.3331 (5.8); 8.3147 (1.2); 7.9024 |
| | | (5.0); 7.8905 (5.3); 7.8829 (5.0); 7.8710 (4.5); 7.8058 (1.5); 7.7896 (3.2); 7.7844 (3.2); |
| | | 7.7684 (6.2); 7.7522 (3.4); 7.7472 (3.6); 7.7312 (1.7); 7.4241 (9.1); 7.4020 (16.0); 7.3801 |
| | | (7.9); 3.3208 (95.0); 2.6756 (2.3); 2.6713 (3.1); 2.6666 (2.3); 2.5245 (10.4); 2.5109 (192.6); |
| | | 2.5067 (372.7); 2.5022 (479.7); 2.4977 (347.6); 2.4934 (171.1); 2.3334 (2.2); 2.3290 (3.0); |
| | | 2.3245 (2.2); 2.0742 (1.0); 0.1460 (1.5); 0.0079 (12.1); −0.0002 (333.2); −0.0084 (12.3);−0.1496 |
| | | (1.5) |

### Biological Examples

Myzus persicae—Oral Test
Solvent: 100 parts by weight acetone

To produce a suitable preparation of active compound, 1 part by weight of active compound is mixed with the stated amount of solvent, and the concentrate is diluted with water to the desired concentration. 50 μl compound solution is filled in microtiter plates and 150 μl IPL41 insect medium (33%+15% sugar) is added to obtain a total volume of 200 μper well. Afterwards the plates are sealed with parafilm through which a mixed population of the green peach aphid (Myzus persicae) can suck on the compound preparation.

After 5 days mortality in % is determined. 100% means all aphids have been killed and 0% means none of the aphids have been killed.

In this test, for example, the following compounds from the preparation examples showed good activity of 100% at an application rate of 20 ppm: I-2-2, I-2-3, I-2-4, I-2-5, I-2-12.

In this test, for example, the following compounds from the preparation examples showed good activity of 90% at an application rate of 20 ppm: I-2-1.

Myzus persicae—spray test
Solvent: 78.0 parts by weight acetone
1.5 parts by weight dimethylformamide
   Emulsifier: alkylarylpolyglycol ether

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Chinese cabbage (Brassica pekinensis) leaf disks infected with all instars of the green peach aphid (Myzus persicae), are sprayed with a preparation of the active ingredient of the desired concentration.

After 5 days mortality in % is determined. 100% means all aphids have been killed and 0% means none of the aphids have been killed.

In this test, for example, the following compounds from the preparation examples showed good activity of 100% at an application rate of 500 g/ha: I-2-5, I-2-6.

Nezara viridula—Spray Test
Solvent: 78.0 parts by weight of acetone
1.5 parts by weight of dimethylformamide
   Emulsifier: alkylarylpolyglycol ether

To produce a suitable preparation of active compound, 1 part by weight of active compound is mixed with the stated amount of solvent, and the concentrate is diluted with water, containing an emulsifier concentration of 1000 ppm, to the desired concentration. Further test concentrations are prepared by dilution with emulsifier containing water.

Barley plants (Hordeum vulgare) infested with larvae of the southern green stink bug (Nezara viridula) are sprayed with a test solution containing the desired concentration of the active ingredient.

After 4 days mortality in % is determined. 100% means all the stink bugs have been killed; 0% means none of the stink bugs have been killed.

In this test, for example, the following compounds from the preparation examples showed good activity of 100% at an application rate of 500 g/ha: I-1-2, I-1-4, I-2-1, I-2-5.

In this test, for example, the following compounds from the preparation examples showed good activity of 90% at an application rate of 500 g/ha: I-1-8, I-1-7, I-1-15.

Nilaparvata lugens—Spray Test
Solvent: 78.0 parts by weight of acetone
1.5 parts by weight of dimethylformamide
   Emulsifier: alkylarylpolyglycol ether

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Rice plants (Oryza sativa) are sprayed with a preparation of the active ingredient of the desired concentration and the plants are infested with the brown planthopper (Nilaparvata lugens).

After 4 days mortality in % is determined. 100% means all planthoppers have been killed and 0% means none of the planthoppers have been killed.

In this test, for example, the following compounds from the preparation examples showed good activity of 100% at an application rate of 500 g/ha: I-1-8.

Phaedon cochleariae—Spray Test
Solvent: 78.0 parts by weight of acetone
1.5 parts by weight of dimethylformamide
   Emulsifier: alkylarylpolyglycol ether

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

Chinese cabbage (Brassica pekinensis) leaf disks are sprayed with a preparation of the active ingredient of the desired concentration. Once dry, the leaf disks are infested with mustard beetle larvae (Phaedon cochleariae).

After 7 days mortality in % is determined. 100% means all beetle larvae have been killed and 0% means none of the beetle larvae have been killed.

In this test, for example, the following compounds from the preparation examples showed good activity of 100% at an application rate of 500 g/ha: I-1-1, I-1-2, I-1-3, I-1-4, I-1-5, I-1-7, I-1-8, I-1-13, I-2-1, I-2-2, I-2-3, I-2-4, I-2-5, I-2-7, I-2-8, I-2-9, I-2-10, I-2-11, I-2-12, I-3-1, I-3-2, I- 5-1, I-5-2.

In this test, for example, the following compounds from the preparation examples showed good activity of 83% at an application rate of 500 g/ha: I-1-12, I-2-6.

In this test, for example, the following compounds from the preparation examples showed good activity of 100% at an application rate of 100 g/ha: I-1-1, I-1-2, I-1-5, I-1-12, I-1-13, I-2-5, I-2-11, I-3-1, I-3-2.

In this test, for example, the following compounds from the preparation examples showed good activity of 83% at an application rate of 100 g/ha: I-2-8, I-2-9, I-2-12, I-5-3.

Tetranychus urticae—Spray Test OP-Resistant
Solvent: 78.0 parts by weight acetone
1.5 parts by weight dimethylformamide
   Emulsifier: alkylarylpolyglycol ether

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water.

French bean (Phaseolus vulgaris) leaf disks infected with all instars of the two spotted spidermite (Tetranychus urticae), are sprayed with a preparation of the active ingredient of the desired concentration.

After 6 days mortality in % is determined. 100% means all spider mites have been killed and 0% means none of the spider mites have been killed.

In this test, for example, the following compounds from the preparation examples showed good activity of 100% at an application rate of 500 g/ha: I-1-5, I-1-7, I-1-8, I-1-10, I-1-12, I-2-8, I-2-11, I-3-1, I-3-2.

In this test, for example, the following compounds from the preparation examples showed good activity of 90% at an application rate of 500 g/ha: I-1-2, I-1-6, I-1-9, I-1-11, I-2-5, I-2-7, I-2-10.

In this test, for example, the following compounds from the preparation examples showed good activity of 70% at an application rate of 500 g/ha: I-1-1, I-2-6, I-2-9, I-2-12.

In this test, for example, the following compounds from the preparation examples showed good activity of 100% at an application rate of 100 g/ha: I-1-8, I-1-12, I-1-13, I-2-8, I-3-1, I-3-2.

In this test, for example, the following compounds from the preparation examples showed good activity of 90% at an application rate of 100 g/ha: I-1-5, I-1-10, I-2-7, I-2-9, I-2-11, I-2-12.

Nezara viridula—Spray Test
Solvent: 52.5 parts by weight of acetone
7 parts by weight of dimethylformamide
   Emulsifier: alkylaryl polyglycolether

To produce a suitable preparation of active compound, 1 part by weight of active compound is mixed with the stated amount of solvent, and the concentrate is diluted with water, containing an emulsifier concentration of 1000 ppm, to the desired concentration. Ammonium salt and/or penetration enhancer in a dosage of 1000 ppm are added to the desired concentration if necessary.

Barley plants (Hordeum vulgare) infested with larvae of the southern green stink bug (Nezara viridula) are sprayed with a test solution containing the desired concentration of the active ingredient.

After 4 days mortality in % is determined. 100% means all the stink bugs have been killed; 0% means none of the stink bugs have been killed.

In this test, for example, the following compounds from the preparation examples showed good activity of 100% at an application rate of 500 g/ha: I-1-1, I-2-1, I-2-5, I-2-6, I-2-7, I-2-8, I-3-1, I-3-2.

In this test, for example, the following compounds from the preparation examples showed good activity of 90% at an application rate of 500 g/ha: I-2-9, I-2-12.

In this test, for example, the following compounds from the preparation examples showed good activity of 100% at an application rate of 100 g/ha: I-1-1, I-2-1, I-2-5, I-3-1, I-3-2.

In this test, for example, the following compounds from the preparation examples showed good activity of 90% at an application rate of 100 g/ha: I-2-8.

Nilaparvata lugens—Spray Test
Solvent: 52.5 parts by weight of acetone
7 parts by weight of dimethylformamide
   Emulsifier: alkylaryl polyglycolether

To produce a suitable preparation of active compound, 1 part by weight of active compound is mixed with the stated amount of solvents and is diluted with water, containing an emulsifier concentration of 1000 ppm, to the desired concentration. Further test concentrations are prepared by dilution with emulsifier containing water. Ammonium salt and/or penetration enhancer in a dosage of 1000 ppm are added to the desired concentration if necessary.

Rice plants (Oryza sativa) are treated by being sprayed with the desired concentration of the active compound and are infested with larvae of the brown planthopper (Nilaparvata lugens).

After 4 days mortality in % is determined. 100% means all planthoppers have been killed and 0% means none of the planthoppers have been killed.

In this test, for example, the following compounds from the preparation examples showed good activity of 100% at an application rate of 500 g/ha: I-3-2.

In this test, for example, the following compounds from the preparation examples showed good activity of 80% at an application rate of 500 g/ha: I-1-1.

1. Compound of formula (I) 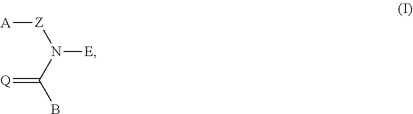
in which
A represents a radical from the group consisting of 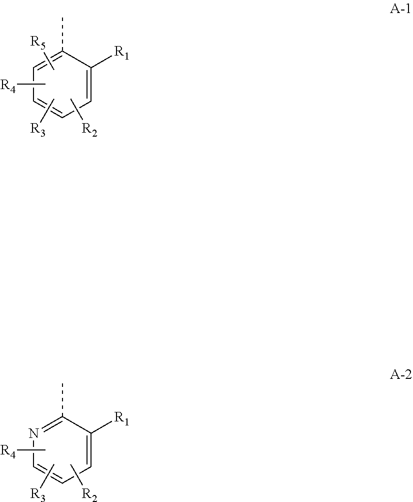
in which the broken line represents the bond to the Z ring,
B represents a radical from the group consisting of 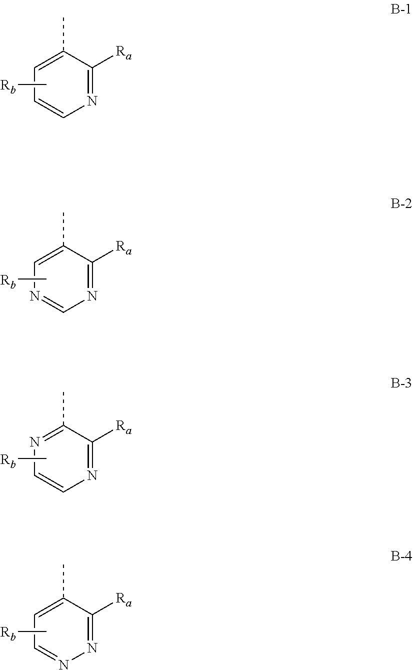
in which the broken line represents the bond to the carbon atom in C=Q,
Z represents a heterocycle from the group consisting of 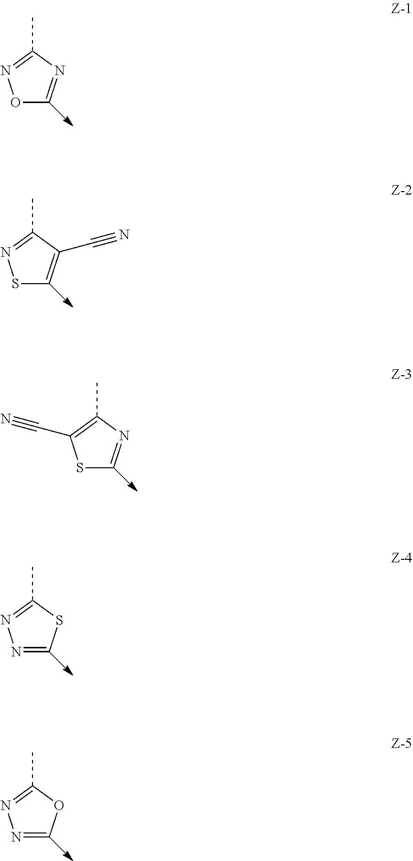
in which the broken line represents the bond to A and the arrow represents the bond to the N(E)C(Q) group,
E is selected from the group consisting of hydrogen, C₁-C₆-alkyl, C₃-C₆-cycloalkyl, C₂-C₆-haloalkyl, C₃-C₆-alkenyl, C₃-C₆-alkynyl, C₃-C₆-cycloalkyl-C₁-C₆-alkyl, cyano-C₁-C₆-alkyl, C₁-C₆-alkylcarbonyl, C₁-C₆-alkoxy-C₁-C₆-alkyl, C₁-C₆-alkoxycarbonyl, a metal ion and an ammonium ion or represents C(═O)—B,
Q represents oxygen or sulfur,
R₁ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₁-C₆-alkylthio, C₁-C₆-haloalkylthio, C₃-C₆-alkenylthio, C₃-C₆-alkynylthio, C₁-C₆-alkylsulphinyl, C₁-C₆-haloalkylsulphinyl, C₁-C₆-alkylsulphonyl and C₁-C₆-haloalkylsulphonyl,
R₂ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₃ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₄ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
R₅ is selected from the group consisting of hydrogen, halogen, cyano, nitro, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl,
Rₐ is selected from the group consisting of halogen, cyano, nitro, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₃-C₆-alkenoxy, C₃-C₆-haloalkenoxy, C₃-C₆-alkynoxy, C₃-C₆-cycloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl and
R_{b} is selected from the group consisting of hydrogen, halogen, nitro, cyano, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₁-C₆-alkylsulphinyl, C₁-C₆-haloalkylsulphinyl, C₁-C₆-alkylsulphonyl and C₁-C₆-haloalkylsulphonyl.

2. Compound of formula (I) according to claim 1 in which A represents A-1, B represents B-3 and R₄, R₅, and R_{b} represent hydrogen.

3. Compound of formula (I) according to claim 1 in which A represents A-1, Z represents Z-2, B represents B-1, Q represents oxygen, and E, R₃, R₄, R₅, and R_{b} represent hydrogen.

4. Compound of formula (I) according to claim 1 in which A represents A-1, Z represents Z-2, B represents B-3, Q represents oxygen, and E, R₃, R₄, R₅, and R_{b} represent hydrogen.

5. Compound of formula (I) according to claim 1 in which A represents A-1, Z represents Z-3, B represents B-1, Q represents oxygen, and E, R₃, R₄, R₅, and R_{b} represent hydrogen.

6. Compound of formula (I) according to claim 1 in which A represents A-1, Z represents Z-3, B represents B-3, Q represents oxygen, and E, R₃, R₄, R₅, and R_{b} represent hydrogen.

7. Compound of formula (I) according to claim 1 in which A represents A-1, Z represents Z-2, B represents B-1, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

8. Compound of formula (I) according to claim 1 in which A represents A-1, Z represents Z-2, B represents B-3, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

9. Compound of formula (I) according to claim 1 in which A represents A-1, Z represents Z-3, B represents B-1, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

10. Compound of formula (I) according to claim 1 in which A represents A-1, Z represents Z-3, B represents B-3, Q represents oxygen, R₁ represents halogen, alkyl or haloalkyl, R₂ represents hydrogen or halogen, Rₐ represents halogen, alkyl or haloalkyl, and E, R₃, R₄, R₅, and R_{b} represent hydrogen.

11. A composition, comprising a content of at least one compound of formula (I) according to claim 1 and one or more customary extenders and/or surfactants.

12. A product comprising a compound of formula (I) according to claim 1 or composition thereof for controlling one or more pests.
